(19) United States
(12) Patent Application Publication
(10) Pub. No.: US 2024/0389564 A1
(43) Pub. Date: Nov. 28, 2024

(54) PEST CONTROL APPARATUS AND METHODS

(71) Applicant: Oxitec Limited, Abingdon (GB)

(72) Inventors: Dyrson De Oliveira ABBADE NETO, Campinas (BR); Zoe Helen BARNES, Faringdon (GB); Amandine COLLADO, Oxford (GB); Matthew James EDWARDS, Abingdon (GB); Grey Kenneth FRANDSEN, Waterford, VA (US); Kevin James GORMAN, Milton Keynes (GB); Kelly Jean MATZEN, Oxford (GB); Neil Ivan MORRISON, Oxford (GB); Neil Frank NAISH, Wantage (GB); Edward Christopher Jack SULSTON, Cirencester (GB); David Francis TREANOR, Abingdon (GB); Mayur Vishnu YADAV, Auchterarder (GB)

(73) Assignee: Oxitec Limited, Abingdon (GB)

(21) Appl. No.: 18/696,885

(22) PCT Filed: Sep. 29, 2022
(86) PCT No.: PCT/US2022/045196

§ 371 Date: Mar. 28, 2024

Related U.S. Application Data
(60) Provisional application No. 63/251,471, filed on Oct. 1, 2021.

(51) Int. Cl. A01K 67/033

(52) U.S. Cl. CPC A01K 67/033

(57) ABSTRACT

Provided herein include apparatuses, devices, kits, and systems for rearing and deploying an arthropod, such as an aquatic insect, e.g., a mosquito. Methods of controlling arthropod populations for arthropod pest control and/or arthropod-home disease control are also disclosed. The compositions and methods disclosed herein are especially useful for high density release of arthropod in an efficient and labor-saving way.

### CROSS-REFERENCE TO RELATED APPLICATION

This application claims priority to U.S. Provisional Patent Application No. 63/251,471 filed Oct. 1, 2022, entitled “PEST CONTROL APPARATUS AND METHODS,” which is herein incorporated by reference in its entirety for all purposes.

### FIELD

The present disclosure relates to apparatuses, kits, and devices for pest population control and methods of use, for example, for rearing and releasing arthropods, such as genetically modified insects, as biological pest control. Associated computer systems, hardware, and software for planning and/or implementing a one-time insect release or periodic releases from one or more devices are also disclosed.

### BACKGROUND

Roughly half of the world's population is at risk of mosquito-borne diseases, with the highest-burden for socioeconomically disadvantaged populations. Urbanization, globalization, climate change, and land-use shifts have each contributed to the re-emergence and expansion of mosquito-borne diseases. For example, dengue incidence has increased >30-fold in the past 50 years and outbreaks of chikungunya, yellow fever, and malaria have increased in size and frequency since 2014. The 2015-2016 Zika virus (ZIKV) epidemic in Latin America and the Caribbean also resulted in hundreds of thousands of infections, resulting in large-scale socioeconomic disruptions. Supply-chain disruptions due to the coronavirus disease 2019 pandemic are expected to increase the number of malaria-related deaths in sub-Saharan Africa in 2020-2021 as well.

There is a critical need for safe, sustainable approaches to reduce the burden of mosquito-borne pathogens. Common mosquito control strategies with chemical insecticides and environmental management are only moderately effective, in part due to resistance arising from physiological (e.g., insecticide resistance) and behavioral changes (e.g., mosquitoes change their blood-feeding times in response to bed nets). Chemical interventions also have unintended effects on non-target insects, including pollinators. As such, current control methods based on insecticides and environmental maintenance have fallen short of eliminating the disease burden.

Scalable, deployable, efficient, and economically viable solutions are needed to control disease propagating vector populations, reduce the burden of insect-borne pathogens, and curb the transmission of related diseases. The present disclosure addresses these and other needs.

### BRIEF SUMMARY

The introduction of sterile insects to an insect population, also known as the sterile insect technique (SIT), can be utilized as a method of controlling unwanted, dangerous or disease propagating insects. In some embodiments of SIT control, male insects are sterilized through irradiation. The sterile males are released into the wild to mate with females. When the sterile males mate with the wild females, no offspring are produced. However, the irradiation dose required to sterilize male insects can cause extensive damage and the irradiated males are often too unfit to compete with wild males. In some embodiments, male pupae after sex sorting can be irradiated, and the sterilized pupae are allowed to emerge to adult in a rearing cage, followed by releasing the adult males in the field.

In addition to SIT, Wolbachia incompatible insect technique (IIT) and other rear-and-release vector control methods can be used to control insect population. In some embodiments, genetically engineered insects containing a self-limiting gene that causes their offspring to die can be used as a biological pesticide. For instance, Release of Insects carrying a Dominant Lethal (RIDL) techniques can be used. The genetically engineered insects do not require irradiation and can be placed in the field at any stage of their life cycle. For instance, in some embodiments, eggs of the genetically engineered insects can be deployed in the field in a device disclosed herein and allowed to emerge to adult in the device from which the adult insects can be released.

Success of the SIT or genetically engineered insects depends on successful and efficient deployment of insects. Efficient deployment can be complicated by various factors including fragility of the insect (especially when insects are reared to adulthood in a mass-rearing facility, transported, and then released), short life expectancy, and limited travel range. For instance, the mosquito is a fragile insect by nature and is easily damaged. The male mosquito also has a short life span (wild-type males living for only about 10 days and irradiated or genetically engineered mosquitoes living for even fewer days). Most house-breeding mosquitoes, such as Aedes aegypti travel no more than about 300 feet in their lifetime from the point of eclosion, that is, the emergence of an adult insect from its pupal case. As such, the use of a mosquito control program to control populations is challenging and certain existing methods require periodic (within days) re-release of sterile males to small geographical areas.

In some aspects, provided herein are scalable and efficient methods for deploying insects such as genetically engineered aquatic arthropod (e.g., insects such as mosquitoes). In some aspects, provided herein is an apparatus for rearing an aquatic arthropod (e.g., insect), comprising: a container comprising a bottom wall and a side wall extending from the bottom wall, and wherein the bottom wall and the side wall are configured to form a chamber for holding a hydrating composition; a receptacle configured to hold eggs of the aquatic arthropod (e.g., insect) and engage the container such that when the hydrating composition is in the chamber, at least a portion of the receptacle contacts the hydrating composition to establish a fluid communication between an inner cavity of the receptacle and the hydrating composition; and an optional housing configured to house the container.

In some embodiments, the housing comprises a housing body and optionally a lid configured to engage the housing body. In some embodiments, the housing body or the container comprises an opening for adding water into the container and the housing or the container does not comprise a lid. In some embodiments, the housing comprises a housing body and a lid engaging the housing body such that the housing has a closed configuration and an open configuration, wherein the housing in the closed configuration encloses the container. In any of the embodiments herein, the lid can be a flap-type lid. In any of the embodiments herein, the lid can be in an open configuration to allow the container to be placed in the housing and/or water to be placed in the container, after which the lid can be sealed into a closed configuration to prevent user and/or predator access (while allowing insects that emerge from the eggs to exit the apparatus when they are ready). In any of the embodiments herein, the lid can be movable relative to the housing body, optionally wherein the lid releasably, rotatably, pivotably, slidably, and/or removably engages the housing body. In any of the embodiments herein, the lid can rotatably engage a first side wall of the housing body and releasably engage a second side wall of the housing body via a releasable locking mechanism.

In any of the embodiments herein, the housing can comprise one or more openings configured to allow exit of an adult of the aquatic insect, optionally wherein the housing body and/or the lid comprises one or more openings configured to allow exit of an adult of the aquatic insect. In any of the embodiments herein, one or more openings can be independently between about 2 mm and about 30 mm in at least one dimension, optionally wherein the one or more openings are between about 3 mm and about 20 mm long and/or between about 3 mm and about 20 mm wide. In any of the embodiments herein, the lid can comprise one or more openings which, together with one or more corresponding structures in the housing and/or the container, provide air communication between the chamber and an outside environment when the housing is in the closed configuration, thereby allowing exit of the adult of the aquatic insect into the outside environment. In any of the embodiments herein, the one or more openings can be configured to prevent rain and/or a predator of the aquatic insect from entering the chamber, optionally wherein the one or more openings are downward inclined from inside to outside of the housing.

In any of the embodiments herein, the housing and/or the container can comprise a temperature insulating material, optionally wherein the housing body, the lid, the bottom wall, and/or the side wall of the container comprises a temperature insulating material. In any of the embodiments herein, the housing can comprise a shell and a temperature insulating filler inside the shell, optionally wherein the shell comprises polypropylene and the filler comprises polyurethane foam. In any of the embodiments herein, the apparatus can comprise a filler between the housing and the container when the container is placed inside the housing, optionally wherein the filler comprises a foam, optionally wherein the foam is a temperature insulating foam disposed on or contacting with an inner surface of the housing and/or an outer surface of the container.

In any of the embodiments herein, the housing, the container, and/or the filler can be configured to maintain an average temperature of between about 10° C. and about 40° C. in the chamber and/or the hydrating composition therein. In any of the embodiments herein, the housing, the container, and/or the filler can be configured to maintain an average temperature of between about 15° C. and about 32° C. in the chamber and/or the hydrating composition therein. In any of the embodiments herein, the housing, the container, and/or the filler can be configured to maintain an average temperature of between about 20° C. and about 28° C. in the chamber and/or the hydrating composition therein.

In any of the embodiments herein, inner surfaces of the housing and/or the container can be configured to be grippable by tarsals of adults of the aquatic insect, optionally wherein the inner surfaces comprise a plurality of indentations and/or protrusions.

In any of the embodiments therein, exterior surfaces of the housing can be configured to prevent rain damage. In any of the embodiments herein, the exterior surfaces of the housing can be coated with a water-resistant material. In any of the embodiments herein, the exterior surfaces of the housing can be coated with a water-resistant resin. In any of the embodiments herein, the exterior surfaces can be configured to prevent solar gain. In any of the embodiments herein, the exterior surfaces can be configured to prevent solar gain by using a pale color (e.g., white) material.

In any of the embodiments herein, the container can releasably and/or removably engage the housing body and/or can be replaceable, optionally wherein the container comprises a recyclable material.

Although in some instances the container and the housing are described separately, it should be appreciated that in any of the embodiments herein, the container and the housing can be one piece that integrates one or more functional components of the container and one or more functional components of the housing. For instance, the container and the housing can be manufactured as one integral piece, e.g., by molding. Alternatively, the container and the housing can be manufactured as separate pieces configured to be assembled together to function as one piece.

In any of the embodiments herein, the container can comprise one or more adapter structures configured to engage the receptacle and/or one or more additional components, optionally where the one or more adapter structures are on the bottom wall. In any of the embodiments herein, the one or more additional components can comprise a water preservative. In some embodiments, the water preservative is provided in a capsule clipped into an adapter structure on the bottom wall. In some embodiments, the water preservative is provided in sachets and is sprinkled into the container and/or the hydrating composition by a user. In some embodiments, the water preservative is formulated as a tablet. In any of the embodiments herein, the water preservative (e.g., formulated as a capsule, sachet, or tablet) can comprise a filler substance and/or an effervescent reagent. In any of the embodiments herein, the water preservative can be formulated with a filler substance. In some embodiments, the filler substance can be an inert material or substance that provides volume and facilitates convenient and accurate dispensing of the water preservative, especially when small quantities of the water preservative are used.

In any of the embodiments herein, the container and/or the hydrating compositions therein can comprise a water preservative, optionally wherein the water preservative comprises sorbic acid or a salt or solvate thereof, or a chlorinated hydroxytriazine or a salt or solvate thereof, or citric acid or a salt or solvate thereof, optionally wherein the water preservative comprises potassium sorbate and/or sodium dichloroisocyanurate and/or citric acid.

In any of the embodiments herein, the filler substance can facilitate the accurate dispensing of the water preservative and formation of tablets/capsules comprising the water preservative. In any of the embodiments herein, the container and/or the hydrating compositions therein can comprise a water preservative formulated as tablets and/or capsules comprising an inert material or substance. In any of the embodiments herein, the filler substance can comprise cellulose, or calcium phosphate, or any combination thereof.

In any of the embodiments herein, the one or more components can comprise a water preservative and an effervescent reagent, optionally wherein the water preservative and an effervescent reagent are formulated as a tablet or capsule. In some embodiments, the effervescent reagent can promote the distribution of the water preservative in the hydrating composition such as water. In any of the embodiments herein, the container and/or the hydrating compositions therein can comprise tablets that can comprise a water preservative and an effervescent reagent, optionally wherein the effervescent reagent comprises sodium bicarbonate, adipic acid, citric acid anhydrous, or any combination thereof. In any of the embodiments herein, the container and/or the hydrating compositions therein can comprise tablets that can comprise a water preservative and an effervescent reagent, optionally wherein the effervescent reagent is adipic acid. In any of the embodiments herein, the container and/or the hydrating compositions therein can comprise tablets that can comprise a water preservative and an effervescent reagent, optionally wherein the effervescent reagent is citric acid anhydrous. In any of the embodiments herein, the container and/or the hydrating compositions therein can comprise a water preservative formulated as tablets, optionally wherein the tablets comprise sodium dichloroisocyanurate, citric acid, sodium bicarbonate, and/or cellulose. In any of the embodiments herein, the container and/or the hydrating compositions therein can comprise a water preservative formulated as tablets, optionally wherein the tablets comprise potassium sorbate, citric acid, and/or sodium bicarbonate. In any of the embodiments herein, the container can comprise a volume indicator of the hydrating composition, optionally wherein the volume indicator comprises a fill line on the side wall of the container. In any of the embodiments herein, the apparatus can be configured to provide a head space between the hydrating composition and the housing in a closed configuration, when the container is placed inside the housing and the hydrating composition is in the chamber.

In any of the embodiments herein, the hydrating composition can comprise or can be water (e.g., purified and/or sterilized water), and the volume of the hydrating composition in the chamber can be between about 200 mL and about 10,000 mL, optionally wherein the volume of the hydrating composition in the chamber is between about 500 mL and about 1,500 mL.

In any of the embodiments herein, the receptacle can be partially or fully submerged in the hydrating composition. In any of the embodiments herein, the receptacle can be clipped into an adapter structure on the bottom wall, tied to the bottom wall, and/or held to the bottom wall by a force from above the receptacle. In any of the embodiments herein, the receptacle can alternatively be mounted on or hung from a lid of the housing and/or a lid of the container, as long as the receptacle can contact the hydrating composition (e.g., for water to wet the eggs, diet, hatch stimulant, and/or water preservative in the receptacle) when the apparatus is in a closed configuration.

In any of the embodiments herein, the receptacle can comprise an egg section for holding eggs of the aquatic insect. In any of the embodiments herein, the receptacle can be configured to provide a fluid communication between the egg section and the hydrating composition when at least a portion of the receptacle contacts the hydrating composition.

In any of the embodiments herein, wherein prior to contacting the hydrating composition, the receptacle can maintain an internal relative humidity between about 50% and about 90%, optionally between about 55% and 75%, thereby maintaining viability of the eggs of the aquatic insect. In any of the embodiments herein, the receptacle can comprise a seal configured to form an airtight seal between the inner cavity and an outside environment, optionally wherein the seal seals the egg section and the diet section from the outside environment. In any of the embodiments herein, the seal can be removable, optionally wherein the seal comprises foil and is manually removable or optionally wherein the seal is dissolvable in the hydrating composition.

In any of the embodiments herein, the receptacle can comprise an internal surface configured to attach to the eggs of the aquatic insect, optionally wherein the internal surface is adhesive or rendered adhesive via spraying and/or painting the internal surface with an adhesive, and optionally wherein the internal surface is on a bottom wall, a side wall, or an internal wall of the receptacle or on an insert or enclosure in the receptacle. In any of the embodiments herein, the internal surface can be on a one-sided or two-sided adhesive strip, optionally wherein the two-sided adhesive strip is affixed to a surface enclosing the egg section.

In any of the embodiments herein, the apparatus can comprise a hatch stimulant, optionally wherein the hatch stimulant is in proximity to the eggs in the receptacle and reduces oxygen potential sufficient to trigger a synchronous larval hatch from the eggs. In any of the embodiments herein, the hatch stimulant may be in the receptacle or outside the receptacle, for instance, in the chamber comprising water (e.g., in a dissolvable capsule clipped to the bottom wall of the container) and/or in a separate receptacle. In any of the embodiments herein, the hatch stimulant may be dissolvable in water and may comprise cysteine, ascorbic acid, or a salt or solvate thereof, or any combination thereof. In any of the embodiments herein, the cysteine concentration in the hydrating composition can be between about 10 mM and about 125 mM, optionally between about 15 mM and about 100 mM. In any of the embodiments herein, the ascorbic acid concentration in the hydrating composition can be between about 1.5 mM and about 40 mM, optionally between about 3 mM and about 30 mM.

In any of the embodiments herein, the apparatus can comprise a diet in the receptacle or outside the receptacle, for instance, in the chamber comprising water (e.g., in a dissolvable capsule clipped to the bottom wall of the container) and/or in a separate receptacle. In any of the embodiments herein, a diet can be provided in the receptacle which comprises a diet section for holding the diet, e.g., for the aquatic insect larvae. In any of the embodiments herein, the receptacle can be configured to provide a fluid communication between the diet section and the hydrating composition when at least a portion of the receptacle contacts the hydrating composition. In any of the embodiments herein, the diet section can be configured to allow the hydrating composition to wet at least a portion of the diet in the diet section. In any of the embodiments herein, the diet section can be configured to allow the hydrating composition to wet the diet in a bottom section of the diet section while maintaining dry diet in a top section of the diet section. In any of the embodiments herein, the diet can be separated from the eggs by a perforated or castellated enclosure.

In any of the embodiments herein, the diet can be formulated to support development of the aquatic insect from egg to larva, to pupa, and/or to adulthood. In any of the embodiments herein, the diet can be formulated to minimize spoilage of the hydrating composition. In any of the embodiments herein, the diet can be formulated to dissolve and/or disintegrate in a controlled manner when contacted with the hydrating composition.

In any of the embodiments herein, the diet can be formulated as pellets, optionally wherein the diet comprises a single pellet type or multiple pellet types, optionally wherein the single pellet type comprises the same or different diet compositions, and optionally wherein the multiple pellet types comprise the same or different diet compositions. In any of the embodiments herein, the diet can be completely or partially coated with one or more layers of coating, optionally wherein the coating is configured to control the rate at which the diet dissolves and/or disintegrates when contacted with the hydrating composition. In any of the embodiments herein, the diet can be completely or partially coated with one or more layers of coating, optionally wherein the coating comprises shellac, carnauba wax, beeswax, or any combination thereof. In any of the embodiments herein, the diet can be completely or partially coated with one or more layers of coating, wherein the coating comprises shellac. In any of the embodiments herein, the diet can be completely or partially coated with one or more layers of coating, wherein the coating comprises carnauba wax. In any of the embodiments herein, the diet can be completely or partially coated with one or more layers of coating, wherein the coating comprises beeswax. In any of the embodiments herein, the diet can be completely or partially coated with one or more layers of shellac, carnauba wax, beeswax, or any combination thereof. In any of the embodiments herein, the diet can be completely or partially coated with one or more layers of shellac, one or more layers of carnauba wax, one or more layers of beeswax, or any combination thereof. In any of the embodiments herein, the diet can be mixed and formulated with an inert material. In any of the embodiments herein, the diet can be mixed and formulated with calcium sulfate hemihydrate (e.g., plaster of Paris). In any of the embodiments herein, the diet can be completely or partially coated with one or more layers of calcium sulfate hemihydrate or inserted into a block comprising calcium sulfate hemihydrate. In any of the embodiments herein, the diet can comprise a plurality of pellets coated by the same coating or by different coatings, optionally wherein the plurality of pellets comprise the same or different diet compositions. In any of the embodiments herein, the diet can be formulated to provide moisture in the receptacle which is sealed, thereby maintaining an internal relative humidity of the sealed receptacle between about 50% and 90%, optionally between about 55% and 75%.

In any of the embodiments herein, the receptacle can be sealed and the sealed receptacle may contain between about 0.01 g and about 0.5 g eggs of the aquatic insect, optionally wherein the sealed receptacle contains between about 0.01 g and about 0.02 g, between about 0.02 g and about 0.03 g, between about 0.03 g and about 0.04 g, between about 0.04 g and about 0.05 g, between about 0.05 g and about 0.06 g, between about 0.06 g and about 0.07 g, between about 0.07 g and about 0.08 g, between about 0.08 g and about 0.09 g, between about 0.09 g and about 0.1 g, between about 0.1 g and about 0.15 g, between about 0.15 g and about 0.2 g, between about 0.2 g and about 0.25 g, between about 0.25 g and about 0.3 g, between about 0.3 g and about 0.35 g, between about 0.35 g and about 0.4 g, between about 0.4 g and about 0.45 g, or between about 0.45 g and about 0.5 g eggs. In any of the embodiments herein, the sealed receptacle can contain between about 1,000 and about 60,000 eggs of the aquatic insect, optionally wherein the sealed receptacle contains between about 1,000 and about 2,000, between about 2,000 and about 3,000, between about 3,000 and about 4,000, between about 4,000 and about 5,000, between about 5,000 and about 6,000, between about 6,000 and about 7,000, between about 7,000 and about 8,000, between about 8,000 and about 9,000, between about 9,000 and about 10,000, between about 10,000 and about 15,000, between about 15,000 and about 20,000, between about 20,000 and about 25,000, between about 25,000 and about 30,000, between about 30,000 and about 35,000, between about 35,000 and about 40,000, between about 40,000 and about 45,000, between about 45,000 and about 50,000, between about 50,000 and about 55,000, or between about 55,000 and about 60,000 eggs of the aquatic insect. In any of the embodiments herein, at least about 30%, at least about 40%, at least about 50%, at least about 60%, at least about 70%, at least about 80%, at least about 90%, or more of the eggs in the receptacle may be hatched.

In any of the embodiments herein, the eggs in the receptacle can be free of a bacterium of the genus Wolbachia, optionally wherein the aquatic insect is a mosquito of the genus Aedes, and optionally wherein the mosquito is Aedes aegypti.

In any of the embodiments herein, the apparatus may contain between about 1,000 and about 50,000 larvae of the aquatic insect, optionally wherein the apparatus contains between about 1,000 and about 2,000, between about 2,000 and about 3,000, between about 3,000 and about 4,000, between about 4,000 and about 5,000, between about 5,000 and about 6,000, between about 6,000 and about 7,000, between about 7,000 and about 8,000, between about 8,000 and about 9,000, between about 9,000 and about 10,000, between about 10,000 and about 15,000, between about 15,000 and about 20,000, between about 20,000 and about 25,000, between about 25,000 and about 30,000, between about 30,000 and about 35,000, between about 35,000 and about 40,000, between about 40,000 and about 45,000, or between about 45,000 and about 50,000 larvae of the aquatic insect. In any of the embodiments herein, at least about 10%, at least about 20%, at least about 30%, at least about 40%, at least about 50%, at least about 60%, at least about 70%, at least about 80%, at least about 90%, or more of the larvae of the aquatic insect can be male.

In any of the embodiments herein, the apparatus may contain between about 500 and about 25,000 hatched male larvae of the aquatic insect, optionally wherein the apparatus contains between about 500 and about 1,000, between about 1,000 and about 1,500, between about 1,500 and about 2,000, between about 2,000 and about 3,000, between about 3,000 and about 4,000, between about 4,000 and about 5,000, between about 5,000 and about 6,000, between about 6,000 and about 7,000, between about 7,000 and about 8,000, between about 8,000 and about 9,000, between about 9,000 and about 10,000, between about 10,000 and about 15,000, between about 15,000 and about 20,000, or between about 20,000 and about 25,000 hatched male larvae of the aquatic insect.

In any of the embodiments herein, the apparatus may be configured to yield between about 250 and about 10,000 adults of the aquatic insect, optionally wherein the apparatus is configured to yield between about 250 and about 500, between about 500 and about 1,000, between about 1,000 and about 1,200, between about 1,200 and about 1,400, between about 1,400 and about 1,600, between about 1,600 and about 1,800, between about 1,800 and about 2,000, between about 2,000 and about 3,000, between about 3,000 and about 4,000, between about 4,000 and about 5,000, between about 5,000 and about 6,000, between about 6,000 and about 7,000, between about 7,000 and about 8,000, between about 8,000 and about 9,000, or between about 9,000 and about 10,000 adults of the aquatic insect. In any of the embodiments herein, at least about 10%, at least about 20%, at least about 30%, at least about 40%, at least about 50%, at least about 60%, at least about 70%, at least about 80%, at least about 90%, at least about 95%, at least about 99%, or 100% of the adults can be males.

In some aspects, disclosed herein is a receptacle comprising: a sealable enclosure comprising an egg section comprising eggs of an aquatic insect, and a diet section comprising diet for the aquatic insect, optionally wherein the egg section and the diet section are separated by a barrier with one or more openings; and a removable seal forming an airtight seal separating the enclosure from an outside environment, wherein when the seal is removed and at least a portion of the receptacle contacts a hydrating composition, the hydrating composition enters the egg section and the diet section and contacts at least a portion of the eggs and at least a portion of the diet.

In some aspects, disclosed herein is a receptacle comprising: (a) a receptacle base comprising: (i) a receptacle bottom wall, (ii) a receptacle side wall extending from the receptacle bottom wall, and (iii) an enclosure, optionally extending from the receptacle bottom wall, wherein the receptacle side wall and/or the enclosure forms an egg section and a diet section within the receptacle base, and wherein the egg section and the diet section are connected; (b) a receptacle lid comprising a plurality of perforations and configured to engage the receptacle base; and optionally (c) a receptacle seal configured to form an airtight seal with the receptacle base and/or the receptacle lid, thereby sealing the egg section and the diet section from an outside environment.

In any of the embodiments herein, the receptacle can be configured to hold eggs of an aquatic insect in the egg section, optionally wherein the eggs are between about 0.06 g and about 0.08 g. In any of the embodiments herein, the receptacle can comprise an adhesive surface on the receptacle side wall, on the receptacle bottom wall, on the enclosure, and/or in the egg section, configured to adhere to eggs of the aquatic insect. In any of the embodiments herein, the receptacle can be configured to hold diet for an aquatic insect in the diet section, optionally wherein the diet is formulated to dissolve and/or disintegrate in a controlled manner when contacted with water. In any of the embodiments herein, the egg section and/or the diet section can be configured to hold a hatch stimulant, optionally wherein the hatch stimulant is cysteine or ascorbic acid or a salt or solvate thereof. In any of the embodiments herein, the egg section and the diet section can be separated by the enclosure which is perforated and/or castellated. In any of the embodiments herein, the enclosure may comprise a plurality of panels extending from the receptacle bottom wall, optionally wherein the enclosure and the receptacle side wall form concentric circles. In any of the embodiments herein, one or more of the plurality of perforations on the receptacle lid may be configured to permit passage of water and one or more larvae of an aquatic insect. In any of the embodiments herein, one or more of the plurality of perforations on the receptacle lid may be independently between about 5 mm and about 50 mm in at least one dimension. In any of the embodiments herein, the receptacle seal can form an airtight seal with the receptacle side wall, and the receptacle lid may be outside, inside, or partially outside and partially inside the receptacle base.

In some aspects, disclosed herein is a receptacle comprising: (a) a receptacle base comprising: (i) a receptacle bottom wall, and (ii) a receptacle side wall extending from the receptacle bottom wall; (b) an enclosure comprising a tubular body and a flange disposed around the tubular body between a bottom end and an upper end of the tubular body, wherein the tubular body comprises a bottom opening, wherein the flange comprises a plurality of perforations, wherein the flange engages the receptacle side wall, the receptacle side wall and/or the tubular body forms an egg section and a diet section within the receptacle base, the egg section and the diet section are connected, and the egg section is connected to a space above the flange via the plurality of perforations on the flange; and optionally (c) a receptacle seal configured to form an airtight seal with the receptacle base, thereby sealing the enclosure, the egg section, and the diet section from an outside environment.

In any of the embodiments herein, the enclosure can be configured to insert into the receptacle base. In any of the embodiments herein, the enclosure can be disposed on or integral to the receptacle bottom wall. In any of the embodiments herein, the upper end of the tubular body can be closed. In any of the embodiments herein, the tubular body can further comprise an upper opening and a closer reversibly closing the upper opening, optionally wherein the closer does not comprise a perforation. In any of the embodiments herein, the receptacle can be configured to hold eggs of an aquatic insect in the egg section, optionally wherein the eggs are between about 0.06 g and about 0.08 g. In any of the embodiments herein, the receptacle can further comprise an adhesive surface on the receptacle side wall, on the receptacle bottom wall, on the tubular body, and/or in the egg section, configured to adhere to eggs of the aquatic insect. In any of the embodiments herein, the receptacle can be configured to hold diet for an aquatic insect in the diet section, optionally wherein the diet is formulated to dissolve and/or disintegrate in a controlled manner when contacted with water. In any of the embodiments herein, the egg section and/or the diet section may be configured to hold a hatch stimulant, optionally wherein the hatch stimulant is cysteine or ascorbic acid or a salt or solvate thereof. In any of the embodiments herein, the egg section and the diet section can be connected via the bottom opening of the tubular body, optionally wherein a bottom section of the tubular body is perforated and/or castellated. In any of the embodiments herein, the tubular body and the receptacle side wall may form concentric circles. In any of the embodiments herein, one or more of the plurality of perforations on the flange may be configured to permit passage of water and a larva of an aquatic insect. In any of the embodiments herein, one or more of the plurality of perforations on the flange may be independently between at least 1 mm in at least one dimension. In any of the embodiments herein, the receptacle seal may form an airtight seal with the receptacle side wall, and the receptacle insert may be outside, inside, or partially outside and partially inside the receptacle base. In any of the embodiments herein, the receptacle seal may form an airtight seal with the receptacle insert, which in turn may form an airtight seal with the receptacle base.

In some aspects, disclosed herein is a method for rearing an aquatic insect, comprising: a) inserting a receptacle into a container, wherein: the container comprises a bottom wall and a side wall extending from the bottom wall, and the bottom wall and the side wall form a chamber for holding water, the receptacle comprises an egg section holding eggs of the aquatic insect and a diet section holding a diet composition for the aquatic insect, and the receptacle is affixed to the bottom wall of the container; b) adding water to the chamber, whereby water enters the receptacle and contacts the eggs and the diet, thereby allowing the eggs to hatch in the receptacle.

In any of the embodiments herein, the receptacle can be inserted into the container, followed by adding water to the chamber to a predetermined level. In any of the embodiments herein, water can be added to the chamber to a predetermined level, followed by inserting the receptacle into the container. In any of the embodiments herein, the egg section can further comprise a hatch stimulant. In any of the embodiments herein, the receptacle can be fully submerged in water. In any of the embodiments herein, water may enter the egg section and the diet section at the same time, or water may enter the egg section prior to entering the diet section, or water may enter the diet section prior to entering the egg section.

In any of the embodiments herein, the diet in the diet section can be wetted prior to hatching of the eggs in the egg section, or at least a portion of the diet in the diet section can be kept dry until after hatching of the eggs in the egg section, until after larvae exit the receptacle, and/or until after adults of the aquatic insect emerge. In any of the embodiments herein, the diet can dissolve and/or disintegrate in a controlled manner in water, optionally at least about 50%, at least about 60%, at least about 70%, at least about 80%, at least about 90%, or at least about 95% of the diet is not dissolved and/or disintegrated in water within the first two, three, four, five, or six days of contacting the water. In any of the embodiments herein, the controlled release of the diet can provide a diet concentration in water that supports development of the aquatic insect and/or reduces spoilage of water compared to a higher diet concentration in water.

In any of the embodiments herein, the method can further comprise placing the container into a housing prior to or after step a) and/or step b), wherein the housing comprises a housing body and a lid configured to engage the housing body such that the housing has a closed configuration and an open configuration. In any of the embodiments herein, the container can be placed in the housing, followed by inserting the receptacle into the container, adding water to the chamber, and closing the housing such that it encloses the container.

In any of the embodiments herein, the housing in the closed configuration can maintain an average temperature of between about 10° C. and about 35° C. in the chamber and/or water therein, optionally wherein the average temperature is between about 15° C. and about 32° C., or optionally between about 20° C. and about 25° C.

In any of the embodiments herein, the lid can comprise one or more openings which, optionally together with one or more corresponding structures in the housing and/or the container, allow exit of adults of the aquatic insect, optionally the one or more openings are downward inclined from inside to outside of the housing.

In any of the embodiments herein, the receptacle, when sealed, may contain between about 0.01 g and about 0.5 g eggs of the aquatic insect prior to water contacting the eggs, optionally wherein the receptacle contains between about 0.01 g and about 0.02 g, between about 0.02 g and about 0.03 g, between about 0.03 g and about 0.04 g, between about 0.04 g and about 0.05 g, between about 0.05 g and about 0.06 g, between about 0.06 g and about 0.07 g, between about 0.07 g and about 0.08 g, between about 0.08 g and about 0.09 g, between about 0.09 g and about 0.1 g, between about 0.1 g and about 0.15 g, between about 0.15 g and about 0.2 g, between about 0.2 g and about 0.25 g, between about 0.25 g and about 0.3 g, between about 0.3 g and about 0.35 g, between about 0.35 g and about 0.4 g, between about 0.4 g and about 0.45 g, or between about 0.45 g and about 0.5 g eggs. In any of the embodiments herein, the receptacle, when sealed, may contain between about 1,000 and about 60,000 eggs, optionally wherein the sealed receptacle contains between about 1,000 and about 2,000, between about 2,000 and about 3,000, between about 3,000 and about 4,000, between about 4,000 and about 5,000, between about 5,000 and about 6,000, between about 6,000 and about 7,000, between about 7,000 and about 8,000, between about 8,000 and about 9,000, between about 9,000 and about 10,000, between about 10,000 and about 15,000, between about 15,000 and about 20,000, between about 20,000 and about 25,000, between about 25,000 and about 30,000, between about 30,000 and about 35,000, between about 35,000 and about 40,000, between about 40,000 and about 45,000, between about 45,000 and about 50,000, between about 50,000 and about 55,000, or between about 55,000 and about 60,000 eggs. In any of the embodiments herein, at least about 30%, at least about 40%, at least about 50%, at least about 60%, at least about 70%, at least about 80%, at least about 90%, or more of the eggs in the receptacle can be hatched.

In any of the embodiments herein, the eggs in the receptacle may be free of a bacterium of the genus Wolbachia, optionally wherein the aquatic insect is a mosquito of the genus Aedes, and optionally wherein the mosquito is Aedes aegypti.

In any of the embodiments herein, the container in the closed housing may contain between about 1,000 and about 50,000 larvae after the eggs hatch, optionally wherein the container in the closed housing contains between about 1,000 and about 2,000, between about 2,000 and about 3,000, between about 3,000 and about 4,000, between about 4,000 and about 5,000, between about 5,000 and about 6,000, between about 6,000 and about 7,000, between about 7,000 and about 8,000, between about 8,000 and about 9,000, between about 9,000 and about 10,000, between about 10,000 and about 15,000, between about 15,000 and about 20,000, between about 20,000 and about 25,000, between about 25,000 and about 30,000, between about 30,000 and about 35,000, between about 35,000 and about 40,000, between about 40,000 and about 45,000, or between about 45,000 and about 50,000 larvae after the eggs hatch, optionally wherein hatching of the eggs is synchronous, and optionally wherein at least about 10%, at least about 20%, at least about 30%, at least about 40%, at least about 50%, at least about 60%, at least about 70%, at least about 80%, at least about 90%, or more of the larvae are male.

In any of the embodiments herein, the container in the closed housing may contain between about 500 and about 25,000 male larvae after the eggs hatch, optionally wherein the container in the closed housing between about 500 and about 1,000, between about 1,000 and about 1,500, between about 1,500 and about 2,000, between about 2,000 and about 3,000, between about 3,000 and about 4,000, between about 4,000 and about 5,000, between about 5,000 and about 6,000, between about 6,000 and about 7,000, between about 7,000 and about 8,000, between about 8,000 and about 9,000, between about 9,000 and about 10,000, between about 10,000 and about 15,000, between about 15,000 and about 20,000, or between about 20,000 and about 25,000 male larvae after the eggs hatch, optionally wherein hatching of the eggs is synchronous.

In any of the embodiments herein, between about 250 and about 10,000 adults of the aquatic insect can emerge in the container in the closed housing and/or exit the closed housing, optionally wherein between about 250 and about 500, between about 500 and about 1,000, between about 1,000 and about 1,200, between about 1,200 and about 1,400, between about 1,400 and about 1,600, between about 1,600 and about 1,800, between about 1,800 and about 2,000, between about 2,000 and about 3,000, between about 3,000 and about 4,000, between about 4,000 and about 5,000, between about 5,000 and about 6,000, between about 6,000 and about 7,000, between about 7,000 and about 8,000, between about 8,000 and about 9,000, or between about 9,000 and about 10,000 adults of the aquatic insect emerge in the container in the closed housing and/or exit the closed housing.

In any of the embodiments herein, at least about 10%, at least about 20%, at least about 30%, at least about 40%, at least about 50%, at least about 60%, at least about 70%, at least about 80%, at least about 90%, at least about 95%, at least about 99%, or 100% of the adults can be males.

In any of the embodiments herein, the adults can be genetically engineered and the method may further comprise allowing the adults to mate with the same species in the wild. In any of the embodiments herein, the adults can comprise a gene expression system capable of producing a protein having a lethal, deleterious, or sterilizing effect, optionally wherein the gene expression system is a sex-specific gene expression system, optionally wherein the gene expression system and/or protein is activated in the absence of a substance, optionally wherein the substance is a chemical ligand, optionally wherein the chemical ligand is tetracycline or an analog or derivative thereof.

In any of the embodiments herein, the method can further comprise allowing the adults to mate with the same species in the wild results in at least some offspring that inherit the gene expression system, thereby suppressing a population of wild insects of said same species. In any of the embodiments herein, suppressing the population of wild insects may result in: (a) reduced transmission of human disease via the population of wild insects; and/or (b) reducing, inhibiting, or eliminating crop damage caused by the population of wild insects.

In some aspects, disclosed herein is a method for rearing an aquatic insect, comprising: a) inserting a receptacle into a container, wherein: the container comprises a bottom wall and a side wall extending from the bottom wall, and the bottom wall and the side wall form a chamber for holding water; the receptacle comprises: a receptacle base comprising: (i) a receptacle bottom wall, (ii) a receptacle side wall extending from the receptacle bottom wall, and (iii) an enclosure, optionally extending from the receptacle bottom wall, wherein the receptacle side wall and/or the enclosure forms an egg section and a diet section within the receptacle base, and wherein the egg section and the diet section are connected, a receptacle lid comprising a plurality of perforations and engaging the receptacle base, and a receptacle seal forming an airtight seal with the receptacle base and/or the receptacle lid, wherein the receptacle seal is removed prior to or after inserting the receptacle into the container; and the receptacle is affixed to the bottom wall of the container; b) adding water to the chamber, whereby the receptacle is submerged in water which enters the receptacle via the plurality of perforations and contacts the eggs and the diet, thereby allowing the eggs to hatch in the receptacle, allowing larvae to exit the receptacle via the plurality of perforations into the chamber, and/or allowing adults of the aquatic insect to emerge in the chamber.

In any of the embodiments herein, the enclosure can extend from the receptacle bottom wall and forms the diet section. In any of the embodiments herein, the egg section can be between the enclosure and the receptacle side wall and may further comprise a hatch stimulant. In any of the embodiments herein, the egg section and the diet section can be separated by the enclosure which is perforated and/or castellated. In any of the embodiments herein, the container can be in a housing comprising a housing body and a lid configured to engage the housing body such that the housing has a closed configuration and an open configuration, wherein the lid comprises one or more openings which, optionally together with one or more corresponding structures in the housing and/or the container, allow exit of adults of the aquatic insect when the housing is in the closed configuration.

In some aspects, disclosed herein is a method for rearing an aquatic insect, comprising: a) inserting a receptacle into a container, wherein: the container comprises a bottom wall and a side wall extending from the bottom wall, and the bottom wall and the side wall form a chamber for holding water; the receptacle comprises: a receptacle base comprising: (i) a receptacle bottom wall, and (ii) a receptacle side wall extending from the receptacle bottom wall, an enclosure comprising a tubular body and a flange disposed around the tubular body between a bottom end and an upper end of the tubular body, wherein the tubular body comprises a bottom opening, wherein the flange comprises a plurality of perforations, and wherein the flange engages the receptacle side wall, the receptacle side wall and/or the tubular body forms an egg section and a diet section within the receptacle base, the egg section and the diet section are connected, and the egg section is connected to a space above the flange via the plurality of perforations on the flange, and a receptacle seal configured to form an airtight seal with the receptacle base, wherein the receptacle seal is removed prior to or after inserting the receptacle into the container; and the receptacle is affixed to the bottom wall of the container; b) adding water to the chamber, whereby the receptacle is submerged in water which enters the receptacle via the plurality of perforations on the flange, contacts the eggs, and, through the bottom opening of the tubular body, contacts the diet, thereby allowing the eggs to hatch in the receptacle, allowing larvae to exit the receptacle via the plurality of perforations on the flange into the chamber, and/or allowing adults of the aquatic insect to emerge in the chamber.

In any of the embodiments herein, the egg section can be between the enclosure and the receptacle side wall and may further comprise a hatch stimulant. In any of the embodiments herein, the egg section and the diet section can be separated by at least a portion of the tubular body which is perforated and/or castellated.

In any of the embodiments herein, the container may be in a housing comprises a housing body and a lid configured to engage the housing body such that the housing has a closed configuration and an open configuration, wherein the lid comprises one or more openings which, optionally together with one or more corresponding structures in the housing and/or the container, allow exit of adults of the aquatic insect when the housing is in the closed configuration.

In any of the embodiments of the apparatus, receptacle, or method herein, the aquatic insect can be a mosquito. In any of the embodiments herein, the mosquito can be of the genus Stegomyia, Aedes, Anopheles, Culex, or Toxorhynchites. In any of the embodiments herein, the mosquito can be Aedes aegypti, Aedes albopictus, Culex pipiens, Culex quinquefasciatus, Anopheles stephensi, Anopheles albimanus or Anopheles gambiae. In any of the embodiments herein, eggs of two or more species of aquatic insects, e.g., two or more species of mosquitoes, can be used in the apparatus, receptacle, or method herein, as long as the two or more species are compatible with each other during development. The two or more species in the wild can be vectors of the same disease or different diseases.

In any of the embodiments herein, the genome of the eggs of the mosquito can comprise a splice control sequence capable of directing sex-specific splicing of a polynucleotide encoding a functional gene product (e.g., a gene product having a lethal or deleterious effect), optionally wherein the functional gene product is a tTAV or homolog thereof, a tTAV2 or homolog thereof, a tTAV3 or homolog thereof, or a tTAF or homolog thereof. In any of the embodiments herein, the genome of the eggs of the mosquito can comprise a transcription control element that controls transcription by the presence or the absence of a chemical ligand, optionally wherein the transcription control element is a tetracycline-responsive element.

In any of the embodiments herein, the sex-specific splicing may result in female-specific lethality of the eggs or larvae, pupae, or adults therefrom. In any of the embodiments herein, the genome of the eggs of the mosquito can comprise a polynucleotide expression system, comprising: a heterologous polynucleotide sequence encoding an RNA for RNA interference (RNAi) and/or a functional protein, the coding sequence of which is defined between a start codon and a stop codon; a promoter capable of initiating transcription in the insect operably linked to the heterologous polynucleotide sequence; and a splice control sequence, which, in cooperation with a spliceosome in the insect or its offspring, is capable of sex-specifically mediating in the insect or its offspring (i) a first splicing of an RNA transcript of the polynucleotide sequence to produce a first spliced mRNA product, which does not comprise a continuous open reading frame extending from the start codon to the stop codon, and (ii) an alternative splicing of said RNA transcript to yield an alternatively spliced mRNA product, which comprises a continuous open reading frame extending from the start codon to the stop codon. In any of the embodiments herein, the splice control sequence can be derived from Actin-4, doublesex (dsx), or transformer (tra). In any of the embodiments herein, the splice control sequence can be derived from an Actin-4 gene, optionally wherein said Actin-4 gene is an Aedes spp. Actin-4 gene, and optionally wherein said Actin-4 gene is an Aedes aegypti Actin-4 gene (AeActin-4).

In any of the embodiments herein, the RNA for RNAi and/or the functional protein can have a lethal or deleterious effect. The functional gene product can comprise a tTAV or homolog thereof, a tTAV2 or homolog thereof, a tTAV3 or homolog thereof, or a tTAF or homolog thereof. The genome of the eggs of the mosquito can comprise two or more of the polynucleotide expression systems, each comprising the same combination of components or a different combination of components. In any of the embodiments herein, the genome of the eggs of the mosquito can comprise a positive feedback loop, for instance, where expression of a functional gene product such as tTAV, tTAV2, tTAV3, or tTAF may drive expression of more of the functional gene product.

In any of the embodiments herein, the genome of the eggs of the mosquito can comprise a male germline gene expression system suitable for conditional expression of an effector gene in an insect male germline, the system comprising: a first expression unit comprising an effector gene and a promoter therefor operably linked thereto; a second expression unit comprising a coding sequence for a transcription factor and an upstream regulatory element operably linked thereto, the transcription factor being capable of acting upon the promoter in the first expression unit to drive expression of the effector gene, the upstream regulatory element comprising a promoter for the transcription factor; and a 5′ UTR adjacent a start site for the transcription factor coding sequence; the upstream regulatory element driving sufficient expression of the transcription factor such that the transcription factor protein in turn drives transcription of the effector gene before meiosis. In any of the embodiments herein, the promoter can be from Beta-2 Tubulin (B2T), matotopetli (topi) or always early (aly). In any of the embodiments herein, the 5′ UTR can be from B2T, a heat shock protein such as hsp83, aly, or topi.

In some embodiments, provided herein is a method comprising: obtaining information of a wild population of an arthropod in a geographic region, optionally wherein obtaining the information comprises forecasting; selecting a location in the geographic region, a date, and/or a time during the day based on the obtained information; placing an apparatus comprising eggs, pupae, larvae, and/or adults of bred arthropods at the selected location, time during the day, and/or date; and allowing adults to emerge from the eggs, pupae, and/or larvae and/or exit the apparatus, wherein the adults mate with the wild population. In some embodiments, the method is for controlling the wild population of the arthropod in the geographic region. In any of the embodiments herein, the arthropod can be an insect, such as a mosquito of the genus Stegomyia, Aedes, Anopheles, Culex, or Toxorhynchites. In any of the embodiments herein, the mosquito can be Aedes aegypti, Aedes albopictus, Culex pipiens, Culex quinquefasciatus, Anopheles stephensi, Anopheles albimanus or Anopheles gambiae.

In any of the embodiments herein, the forecasting can comprise using: information regarding the arthropod; information regarding conditions in and/or outside the apparatus; information regarding weather and climate in the geographic region; terrain information in the geographic region; and/or information regarding human population and/or activity in the geographic region. In any of the embodiments herein, the information regarding the arthropod can comprise: hatching time, length of the larval stage, length of the pupal stage, time from hatching to adulthood, adult life expectancy, and/or flight range of the species of the arthropod, one or more diseases transmitted or borne by the species of the arthropod, and/or crop or livestock damage caused by the species of the arthropod, and the information can optionally comprise a reference or an estimate, such as a reference or estimated value of any of the aforementioned parameters. In any of the embodiments herein, the information regarding the arthropod can comprise: the size of the wild population, the density of the wild population, and/or the distribution of the wild population in the geographic region, and/or the population size, density, and/or distribution of one or more predators of the arthropod in the geographic region, and the information can optionally comprise historical information and/or information obtained at the time of the forecasting. In any of the embodiments herein, the information regarding climate can comprise information regarding season, temperature, rainfall, humidity, and/or wind conditions in the geographic region. In any of the embodiments herein, the information can comprise historical information, a reference or an estimate, and/or information obtained at the time of the forecasting. In any of the embodiments herein, the information can comprise annual or monthly mean temperatures and precipitations in the geographic region. In any of the embodiments herein, the terrain information can comprise information regarding the amount and/or distribution of water bodies in the geographic region. In any of the embodiments herein, the water bodies can comprise stagnant water, rivers, lakes, ponds, tanks, reservoirs, and/or water treatment facilities. In any of the embodiments herein, the terrain information can comprise a reference or an estimate, such as a reference or estimated value of any of the aforementioned parameters. In any of the embodiments herein, the information regarding human population and/or activity can comprise information regarding the size, density, distribution, demographic information, and/or personal information of the human population in the geographic region. In any of the embodiments herein, the information can comprise historical information, a reference or an estimate, and/or information obtained at the time of the forecasting.

In any of the embodiments herein, obtaining any of the aforementioned information and/or the forecasting can be performed using one or more devices automatically, semi-automatically, or manually. In any of the embodiments herein, obtaining any of the aforementioned information and/or the forecasting can be performed using information obtained from one or more sources. In any of the embodiments herein, the one or more sources can comprise a local source in one or more devices configured to perform the forecasting and/or one or more internet servers.

In any of the embodiments herein, the forecasting can be for about a week, about two weeks, about three weeks, about four weeks, or longer from the time of forecasting, e.g., the time of placing the apparatus at the location, inserting the egg receptacle in the apparatus, or wetting the eggs in the receptacle.

In any of the embodiments herein, the geographic region can be selected using one or more devices, optionally wherein the geographic region is regular or irregular in shape. In any of the embodiments herein, the geographic region can be selected using the same device(s) for performing the forecast. In any of the embodiments herein, the geographic region can be selected using device(s) different from device(s) for performing the forecast. In any of the embodiments herein, the selected geographic region can be provided to device(s) for performing the forecast based on the selected geographic region.

In any of the embodiments herein, the location in the geographic region can be selected using one or more devices. In any of the embodiments herein, the location can be selected using the same device(s) for performing the forecast and/or the same device(s) for selecting the geographic region. In any of the embodiments herein, the location can be selected using different from device(s) for performing the forecast and/or device(s) for selecting the geographic region. In any of the embodiments herein, the forecasted information can be provided to device(s) for selecting the location in the geographic region.

In any of the embodiments herein, a plurality of locations can be selected in the geographic region, and one or more of the apparatuses disclosed herein can be placed at each location at the same time or at different times (e.g., on different dates and/or at different times during the day). In any of the embodiments herein, the plurality of locations can form a grid in which each location is a node. In any of the embodiments herein, the grid can comprise a regular grid and/or an irregular grid. In any of the embodiments herein, the grid can be a regular grid or an irregular grid. In any of the embodiments herein, one or more of the same apparatus (e.g., apparatuses having the same number of eggs and same configuration of the egg receptacles) can be placed at each of the plurality of locations. In any of the embodiments herein, one or more different apparatuses (e.g., apparatuses having different numbers of eggs and/or different configurations of the egg receptacles) can be placed at each of the plurality of locations. The one or more apparatuses can independently be any apparatus disclosed herein.

In any of the embodiments herein, the method can comprise using ground-based data gathering to obtain population density data of the arthropod population in and/or around the geographical region. In any of the embodiments herein, the method can comprise using the obtained population density data for the forecast.

In any of the embodiments herein, the method can comprise delivering the apparatus comprising eggs, pupae, larvae, and/or adults of bred arthropods at or near the location. In any of the embodiments herein, the apparatus can be delivered at or near the location using a ground distribution vehicle or an aerial craft, optionally wherein the aerial craft is a piloted aircraft or a pilotless drone. In any of the embodiments herein, the method can comprise providing information of the apparatus to a user to place the apparatus. In any of the embodiments herein, the information of the apparatus can comprise tracking formation. In any of the embodiments herein, the tracking formation can be provided on a map (e.g., in an app disclosed herein) on a device of the user. In any of the embodiments herein, the information of the apparatus can comprise expiry information of the apparatus, such as expiry information of eggs in an egg receptacle. In any of the embodiments herein, the method can comprise providing information of one or more apparatuses that have been placed in the geographic region.

In some embodiments, provided herein is a non-transitory computer-readable medium comprising processor-executable instructions to cause a processor to: obtain information of a wild population of an arthropod in a geographic region, optionally wherein the processor-executable instructions cause the processor to forecast the information; provide instruction or recommendation to a user for placing an apparatus comprising eggs of bred arthropods at one or more locations in the geographic region based on the forecasted information, wherein adults of the bred arthropod emerge from the eggs, pupae, and/or larvae and/or exit the apparatus to mate with the wild population.

In any of the embodiments herein, the method and/or the non-transitory computer-readable medium can be for use in controlling the wild population of the arthropod in the geographic region. In any of the embodiments herein, the adults that exit the apparatus can comprise, consist essentially of, or consist of males. In any of the embodiments herein, the adults that exit the apparatus can comprise, consist essentially of, or consist of females. In any of the embodiments herein, the adults that exit the apparatus can comprise both males and females. In any of the embodiments herein, the adults that exit the apparatus can comprise, consist essentially of, or consist of sterile male insects and/or sterile female insects, Wolbachia-infected male insects and/or Wolbachia-infected female insects, and/or genetically engineered male insects and/or genetically engineered female insects. In any of the embodiments herein, the adults that exit the apparatus can comprise, consist essentially of, or consist of genetically engineered insects that mate with wild insects of the same species of the opposite sex; sterile or sterilized insects that mate with wild insects of the same species of the opposite sex, optionally wherein the insects are sterilized using radiation; and/or insects that carry a bacterium of the genus Wolbachia that mate with wild insects of the same species of the opposite sex, optionally wherein the insects are infected with Wolbachia pipientis. In any of the embodiments herein, the adults that exit the apparatus can comprise, consist essentially of, or consist of male insects that are not genetically engineered but are irradiated and/or sterilized. In any of the embodiments herein, the adults that exit the apparatus can comprise, consist essentially of, or consist of genetically engineered male insects that mate with wild female insects of the same species, thereby control the wild population in the geographic region. In any of the embodiments herein, the arthropod can be a mosquito of the genus Aedes, Anopheles, or Culex. In any of the embodiments herein, the apparatus may contain eggs of the bred arthropods (e.g., eggs of genetically engineered, radiation-sterilized, and/or Wolbachia-infected mosquitoes) and no pupae, larvae, or adults of the bred arthropods.

### BRIEF DESCRIPTION OF THE DRAWINGS

The drawings illustrate certain embodiments of the features and advantages of this disclosure. These embodiments are not intended to limit the scope of the appended claims in any manner.

FIG. 1 shows an exemplary apparatus 100 comprising a housing 120, an optional container 140, and a receptacle 160 comprising eggs of an aquatic arthropod (e.g., insect). The container is optional in that it may be integrated with the housing as a single piece that is assembled from multiple parts or integrally manufactured (e.g., molded as one piece). In cases where a separate container is used, the container can be inserted into the housing.

FIG. 2 shows an exemplary receptacle 260 comprising a seal 261, eggs of an aquatic arthropod (e.g., insect) (not shown) in an egg section 265, and optionally diet 268 in a diet section 267. The receptacle can be inserted into the container/housing. The seal may be removed before inserting the receptacle or after the receptacle contains water (e.g., the seal may be dissolved when submerged in water). The eggs can include eggs of genetically engineered mosquitoes, radiation-sterilized mosquitoes, and/or Wolbachia-infected mosquitoes.

FIG. 3 shows inserting an exemplary receptacle 360 into the container 340. The receptacle may be affixed to the bottom wall of the container via an adapter structure 348. Additional components such as water preservatives 352 may be provided in the container, e.g., affixed to the bottom wall of the container via adapter structures 350.

FIG. 4 shows addition of water an exemplary container 440 to submerge the receptacle 460 and the eggs and at least a portion of the diet in the receptacle in water, thereby creating an aquatic environment capable of rearing the aquatic insect in the container.

FIG. 5 shows an exemplary housing lid or cover 121 in an open configuration. The housing lid 121 can be displaced from an open configuration to a closed configuration. The lid may rotatably engage a first side wall of the housing body and releasably engage a second side wall of the housing body via a releasable locking mechanism 527.

FIG. 6A shows an exemplary housing 620 in a closed configuration. An exemplary lid 621 comprises openings 623 which allow adults of the aquatic insect to exit the apparatus. FIG. 6B shows an exemplary insert 629 being inserted into the housing 620 in a closed configuration, which may release the releasable locking mechanism.

FIG. 7 shows an exemplary apparatus 700 with the housing 720 in an open configuration. A container 740 placed inside the housing contains water preservative capsules 752 and a receptacle 760 submerged in water 770.

FIG. 8A shows parts of an exemplary receptacle 860 in an unassembled configuration, comprising a receptacle base 880 forming an egg section 865 and a diet section 867 connected through slits 869, an insert 868, a receptacle lid 863 with perforations 870, and a seal 861. FIG. 8B shows parts of an exemplary receptacle 860 in an assembled configuration. The receptacle base 880 is sealed by a seal 861, which is depicted as transparent to show additional parts.

FIG. 9A shows parts of an exemplary receptacle 960 in an unassembled configuration, comprising a seal 961, receptacle lid 963, an enclosure 966 comprising a tubular body 972 and flange 974, an insert 968, and a receptacle base 980. FIG. 9B shows parts of an exemplary receptacle 960 in an assembled configuration.

FIG. 10 illustrates an example of a computing device 1000, in accordance with an embodiment. In some embodiments, the computing device is configured to be in the disclosed systems and is configured to perform the operational methods associated with the systems disclosed herein.

FIG. 11A shows assembling steps for an exemplary rearing device, e.g., for mosquitoes.

FIG. 11B shows an exemplary portable light-weight rearing device, e.g., for mosquitoes.

### DETAILED DESCRIPTION

The following detailed description is merely exemplary in nature and is not intended to limit the present disclosure or the application and uses thereof. Furthermore, there is no intention to be bound by any expressed or implied theory presented in the preceding Field, Background, Brief Summary, or Brief Description of the Drawings section, or the following detailed description. Specific embodiments of the present disclosure are in some cases described with reference to the figures, wherein like reference numbers indicate identical or functionally similar elements.

The following sections describe various non-limiting examples of systems and methods for insect rearing and/or deployment of the insects, including aquatic insects such as mosquitoes. It should be appreciated that “aquatic insect” refers to insects that have a lifecycle that includes an aquatic stage and is not limited to insects that are aquatic insects as adults. For example, examples according to this disclosure may be used to rear and deploy insects (such as Aedes aegypti, Aedes albopictus, or Anopheles gambiae mosquitoes), including genetically modified populations thereof that can act as a “living insecticide” or “biological pesticide” for insects of the same species in the wild. The genetically modified populations can be generated using any suitable genetic engineering method, including but not limited to a method involving a transposable element (e.g., piggyBac or Sleeping Beauty), meganuclease, zinc finger nuclease (ZFN), site specific recombinase (e.g., a Cre-LoxP or Flp-FRT system), a transcription activator-like effector nuclease (TALEN), a CRISPR (e.g., a CRISPR/Cas system such as CRISPR/Cas9a/b/c or CRISPR/Cas12a/b or CRISPR/Cas13a/b/c/d), or any combination thereof (e.g., a transposon-associated CRISPR-Cas system such as CRISPR-transposons).

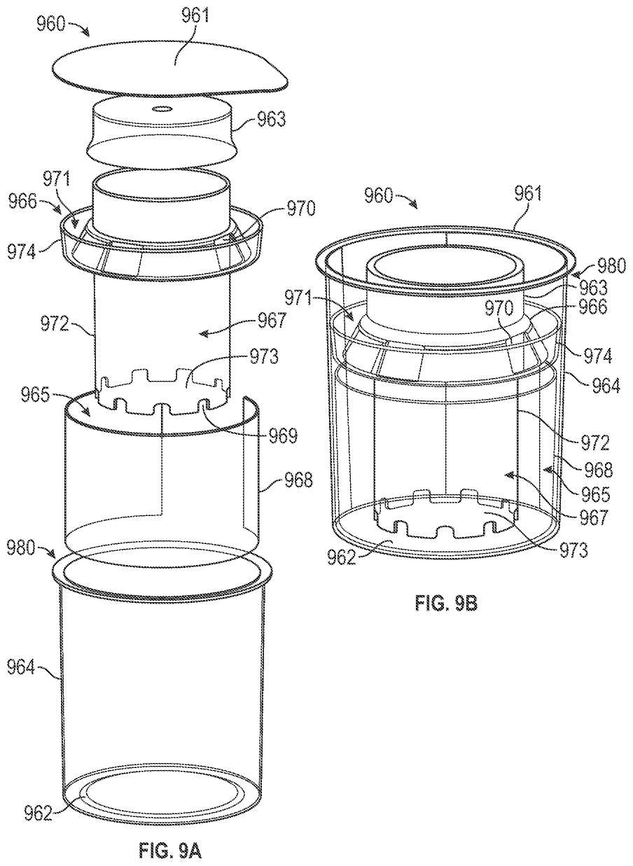

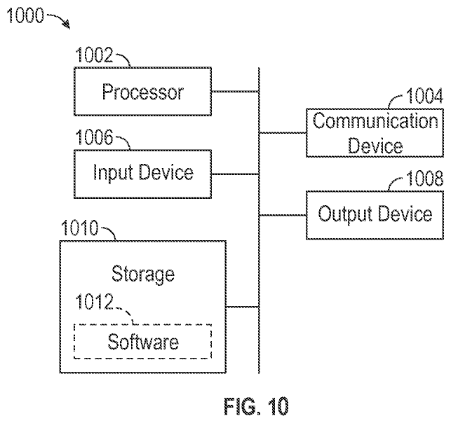

In some embodiments, the present disclosure relates to apparatuses, kits, and devices for insects, for instance, for rearing an aquatic insect from eggs, larvae, and/or pupae until they reach adulthood, at which time the insects can exit or be released from a device disclosed herein for controlling wild populations of the insect of the same species in a geographical area. In some embodiments, the apparatuses, kits, and devices are used to synchronize hatching of eggs, optionally thereby synchronizing development of the insects after hatching to achieve controlled (e.g., synchronized) release, for instance, within a period of time from installing a receptacle comprising insect eggs in the device and/or with a desired density of adult insects to be released per unit device and/or per unit area in a target geographical area. In other embodiments, hatching of eggs in a device disclosed herein is not synchronized. As such, using a device disclosed herein, hatching of eggs may but does not need to be synchronized. The device may be utilized in support of a self-limiting aquatic insect control program, such as one that involves only allowing survival of male offspring. The device may be utilized in support of any insect control program involving rearing and deployment of an aquatic insect, including ones involving deployment of males only, ones involving deployment of females only, and/or ones involving deployment of both males and females in any ratio. Utilization of a device disclosed herein may comprise using a single unit of a device or multiple units of the same device or different devices in any suitable temporal and/or spatial combination. For instance, the device(s) may be used over a small geographical area or a large geographical area, and adult insects may be released once (e.g., at a single time point or within a single time window) or multiple times (e.g., at multiple time points or within multiple time windows) over a period of time, e.g., insect deployment can be repeated in the geographical area and the repeated releases can be periodic. Furthermore, devices of different designs may be used in any suitable temporal and/or spatial combination. For instance, one or more units of a device comprising a receptacle of a first design (e.g., as shown in FIGS. 8A-8B) and one or more units of a device comprising a receptacle of a second design (e.g., as shown in FIGS. 9A-9B) may be used simultaneously or in tandem for the same geographical area and/or may be used in different sub-areas of a larger area (e.g., depending on estimated mosquito population sizes). The device may also be used in conjunction with one or more other insect control programs in any suitable order, including but not limited to a sterile insect technique (SIT) program, the introduction of a parasite, pathogen (e.g., viruses, bacteria such as Wolbachia pipientis and/or other Wolbachia bacteria, fungi, protozoa, nematodes and microsporidia), and/or predator to target mosquitoes, or any combination thereof, for instance, a part of an integrated pest management (IPM) program.

The device and its various embodiments described in greater detail below, provide a cost effective and repeatable release solution for many different insect species, particularly aquatic insects such as mosquitoes. In some embodiments, the device and/or its components are utilized to hatch eggs, mature the juvenile insects (such as insect larvae or pupae in water) to adults, keep them safe in a release site, and/or release them efficiently, precisely, in volume, at a high density, and with minimal manpower after installation. Optionally, the device and/or its components can be utilized to feed adults that emerge in the device. With the high volume/density device disclosed herein, insects, including mosquitoes, may be matured and released in a much more efficient and labor-saving way than current methods. In particular embodiments, the device described herein provide a cost effective and repeatable release solution for deploying male insects such as mosquitoes at a high density, e.g., at least about 100, at least about 250, at least about 500, at least about 750, at least about 1,000, at least about 1,250, at least about 1,300, at least about 1,350, at least about 1,400, at least about 1,450, at least about 1,500, or more than 1,500 male mosquitoes per unit device.

Also provided herein are computer systems, hardware, and software, including computer programs and applications, that can be used to implement a method of the present disclosure. In some aspects, provided herein are computer systems, hardware, and software that can be used in connection with devices and methods of rearing and/or releasing an aquatic insect, for instance, by interfacing with a potential user or a user of the pest control apparatus, device, kit, or system disclosed herein. In some aspects, the computer systems, hardware, and software can be used to facilitate and/or allow a user to efficiently rear and/or release insects, such as aquatic insects including mosquitoes. In some aspects, the computer systems, hardware, and software can be used to facilitate and/or allow a user to efficiently implement and/or manage an integrated pest management program.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. APPARATUS AND COMPONENTS THEREOF

In some aspects, disclosed herein is an apparatus for rearing an arthropod, such as an aquatic arthropod, including an aquatic insect. In some embodiments, the apparatus can also be used to deploy adults of an aquatic insect that have emerged in the apparatus, for instance, into a geographical area in which the apparatus has been placed, in order to control a wild population of the aquatic insect in the geographical area.

In some embodiments, the apparatus comprises two or more components or modules that are individually provided. For instance, individual components or modules can be manufactured, packaged, and/or obtained or shipped separately, and a user can assemble the individual components into a device. In some embodiments, the apparatus comprises two or more components or modules that are provided together, optionally pre-assembled or manufactured (e.g., molded) as one piece. For instance, the components or modules can be manufactured, packaged, shipped, and/or obtained together. In some embodiments, two or more or all of the components or modules can be manufactured, packaged, shipped, and/or obtained in one piece. In some embodiments, the components or modules can be integral to one another, e.g., they can be manufactured as one piece without requiring a user to assemble them. In some embodiments, the components or modules can releasably and/or removably engage one another and/or one or more other components or modules. A user can disassemble the components or modules and/or assemble them with other component(s) or module(s) as needed.

In some embodiments, the apparatus comprises: a housing; a container configured to be placed inside the housing, wherein the container comprises a bottom wall and a side wall extending from the bottom wall, and wherein the bottom wall and the side wall are configured to form a chamber for holding a hydrating composition; and a receptacle configured to hold eggs of the aquatic insect and engage the container such that when the hydrating composition is in the chamber, at least a portion of the receptacle contacts the hydrating composition to establish a fluid communication between an inner cavity of the receptacle and the hydrating composition. In some embodiments, the housing is referred to as an outer and the container is referred to as a liner. In some embodiments, the housing (e.g. an outer vessel) is configured to house the various components of the apparatus.

One or more features of the housing described in the present disclosure can be provided on or integrated into the container, or vice versa. For instance, the housing and the container can be combined into a single piece. Optionally, the housing and the container can be manufactured and/or packaged as one piece. Alternatively, the housing and the container can be separately manufactured and/or packaged but are configured to be assembled into one piece for use. As such, disclosed herein in some aspects is an apparatus for rearing an aquatic insect, comprising: a housing comprising a bottom wall and a side wall extending from the bottom wall, and wherein the bottom wall and the side wall are configured to form a chamber for holding a hydrating composition; a receptacle configured to hold eggs of the aquatic insect and engage the housing such that when the hydrating composition is in the chamber, at least a portion of the receptacle contacts the hydrating composition to establish a fluid communication between an inner cavity of the receptacle and the hydrating composition. An optional additional housing configured to house the various components of the apparatus including the housing may be used but is not necessary.

The apparatus may take any of a variety of three dimensional forms. In some embodiments, the apparatus and/or housing may generally take the form of a sphere, cube, cylinder, or prism. In some embodiments, the apparatus and/or housing may take the form of a rectangular, hexagonal, or any other type of prism.

In some embodiments, the apparatus and/or the housing is configured to be mounted or hung. In some embodiments, the apparatus and/or the housing is configured to be mounted on or hung from a post (e.g., a lamp post), tree, wall, building, fence, or other supporting object. In some embodiments, the apparatus and/or the housing is configured to be stably positioned on generally flat ground or a generally flat surface. In some embodiments, the apparatus and/or the housing is configured to be free-standing. In some embodiments, the apparatus and/or the housing is configured to be mobile, e.g., it may be carried or otherwise transported from one location or another. In some embodiments, the housing is configured to have lightweight and robust construction, allowing ease of distribution and repeated use. In some embodiments, the housing is configured to be used for about 1 month, 2 months, 6 months, 1 year, 2 years, 3 years, 4 years, 5 years or longer.

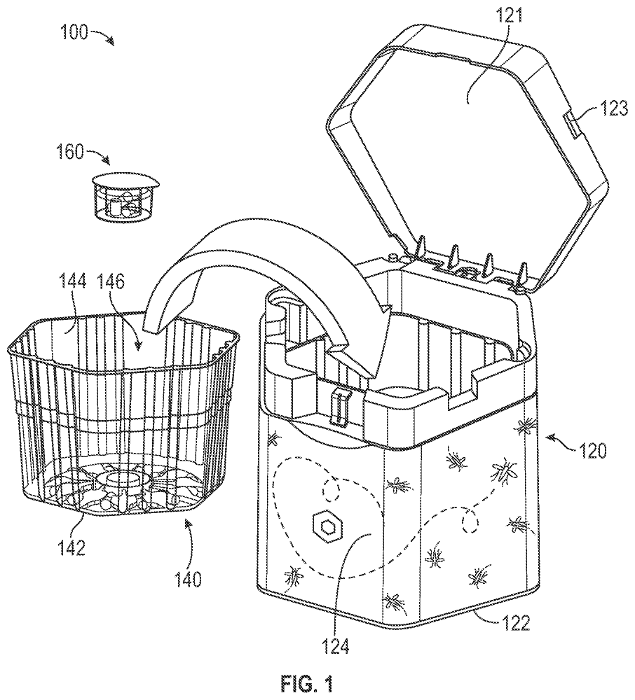

Referring now to FIG. 1, disclosed herein in some aspects is an apparatus 100 for rearing an arthropod (e.g., insect), including arthropods that lives some portion of their life cycle in water. In some aspects, the apparatus comprises a housing 120 (e.g., an outer vessel or box, sometimes referred to as “outer” for simplicity). The housing generally comprises a housing lid or cover 121 and a housing body comprising a housing bottom wall 122 and a housing side wall 124 extending from the housing bottom wall. The housing body may but in some aspects may not need to comprise a bottom wall (e.g., where the container comprises a bottom wall which after being placed in the housing can function as a bottom wall for the housing). In some aspects, the apparatus comprises a container 140 (e.g., an inner vessel or liner) which is placed inside the housing, or is separately provided and configured to be placed in the housing. The container in general comprises a bottom wall 142 and a side wall 144 extending from the bottom wall, and the bottom wall and the side wall form or are configured to form a chamber 146 for holding a hydrating composition, e.g., water or other compositions including a water-based slurry or the like that provides a flowable aquatic environment for the insect.

In some aspects, housing lid or cover 121 can function as a container lid or cover, housing bottom wall 122 can function as container bottom wall 142 and housing side wall 126 can function as container side wall 144, and/or the housing bottom wall and housing side wall can form chamber 146. As such, the housing and the container can be one single piece.

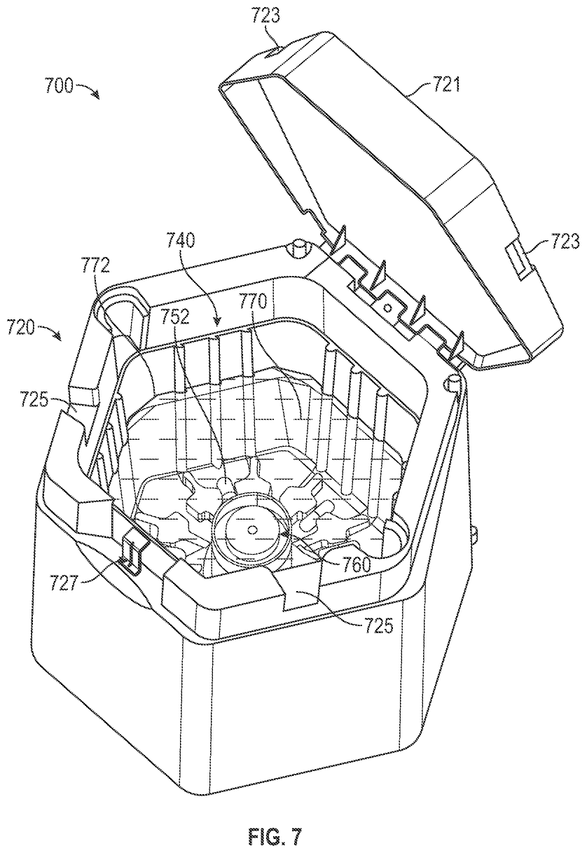

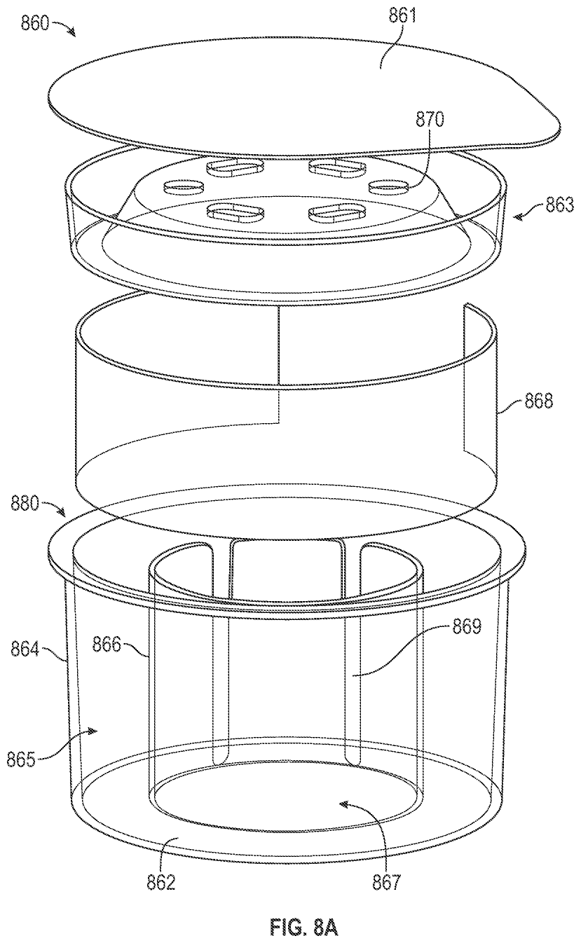

The housing 120 may be any suitable system that can receive and house the receptacle and the hydrating composition and/or a container in which the receptacle and the hydrating composition may be added. The housing lid or cover 121 can be configured to engage the housing body (e.g., housing side wall 124) such that the housing has a closed configuration (e.g., as shown in FIG. 6) and an open configuration (e.g., as shown in FIG. 1 or FIG. 5). The housing in the closed configuration can enclose the container and the receptacle and the hydrating composition therein. In general the lid is movable relative to the housing body and allows addition and/or removal of the container, the hydrating composition, and/or the receptacle. For instance, after adult insects emerged from one unit of the receptacle are released, the container, the hydrating composition, and/or the receptacle can be replaced by a fresh set and the housing can be reused while the used container, hydrating composition, and/or receptacle can be discarded and/or recycled. Thus in some aspects, the container releasably and/or removably engages the housing body and is replaceable. In some aspects, the container comprises a recyclable material and is optionally entirely made of recyclable material(s). The lid can releasably, rotatably, pivotably, slidably, and/or removably engage the housing body. In some aspects, the lid rotatably engages a first side wall of the housing body and releasably engages a second side wall of the housing body via a releasable locking mechanism (e.g., locking mechanism 527 as shown in FIG. 5). As shown in FIG. 1, lid 121 is rotatable around an axis on a first side wall of the hexagonal housing body and can releasably engage a second side wall of the housing body on the opposite side of the first side wall. The lid and the housing body can be separately provided and then releasably or fixedly assembled to form the housing, or can be manufactured as a single piece.

In some embodiments, the lid rotatably engages a first side wall of the housing body and releasably engages a second side wall of the housing body via a releasable locking mechanism. In some embodiments, the releasable locking mechanism includes an access slot. In some embodiments, the access slot forms a narrow opening between the housing body and the lid when the housing has a closed configuration. The access slot is configured such that an object inserted into the access slot may contact and release the releasable locking mechanism, causing the housing to move from a closed configuration to an open configuration. In some embodiments, the object inserted into the access slot is a rigid and generally flat object (e.g., a key or a card such as a rectangular card).

In some embodiments, the housing is configured to allow release of an adult of the aquatic insect (e.g., an adult mosquito). In some embodiments, the housing comprises one or more openings configured to allow exit of an adult of the aquatic insect (e.g. an adult mosquito). In some embodiments, the housing body and/or the lid comprises one or more openings configured to allow exit of an adult of the aquatic insect.

In some embodiments, the lid comprises one or more openings which provide air communication between the chamber and an outside environment when the housing is in the closed configuration, thereby allowing exit of the adult of the aquatic insect into the outside environment. In some embodiments, the chamber is enclosed at least in part by the container and/or the housing, such as the housing lid when the housing is in a closed configuration. In some embodiments, the lid comprises one or more openings which, together with one or more corresponding structures in the housing and/or the container, provide air communication between the chamber and an outside environment when the housing is in the closed configuration, thereby allowing exit of the adult of the aquatic insect into the outside environment. In some embodiments, the one or more openings are each independently between about 2 mm and about 30 mm in at least one dimension. In some embodiments, the one or more openings are between about 3 mm and about 20 mm long and/or between about 3 mm and about 20 mm wide.

In some embodiments, the one or more openings are configured to prevent rain and/or a predator of the aquatic insect from entering the chamber. In some embodiments, the one or more openings are configured to prevent substances, objects, organisms, or parts thereof (e.g. plants, leaves, pollen, dirt, debris, litter, animals, parasites, single-celled organisms), from the exterior of the apparatus from entering the chamber. In some embodiments, the one or more openings are configured to minimize evaporation of water from the apparatus. In some embodiments, the one or more openings are located on a side of one or more of the apparatus, housing, housing body, or lid. In some embodiments, the openings are downward inclined from the inside to the outside of the housing.

Referring to FIG. 1, the housing 120 comprises one or more openings 123 configured to allow exit of an adult of the aquatic insect once it emerges inside the closed housing. FIG. 1 shows one or more openings 123 are at least partially provided in lid 121. In some aspects, the one or more openings together with one or more corresponding structures in the housing and/or the container provide air communication between the chamber and an outside environment, such as a geographical area in which the aquatic insects are to be deployed. In some aspects, the apparatus comprises two exit slots 123 incorporated into lid 121, e.g., one each on opposite sides of the hexagonal lid. The exit slots combined with additional features molded into the housing body and/or the container allow adult insects such as adult aquatic insects (e.g., mosquitoes) to escape. In some aspects, the one or more openings configured to allow exit of an adult of the aquatic insect can be provided in the housing body, or by the combination of the lid and the housing body (for instance, the lid can comprise a notch and the housing body comprises another notch at a corresponding position such that when the housing is closed, the notches form an opening).

The one or more openings 123 such as exit slots can be of any suitable size, shape, or configuration. For instance, when the housing is in the closed configuration, exit slots of a suitable size and shape allow the adult of the aquatic insect to exit into the outside environment, whereas rain and animals, including other insects and predators of the aquatic insect, can be precluded from entering the closed housing. In particular examples, predators of mosquitoes (e.g., a dragonfly, a damselfly, a predatory mosquito, e.g., in the genus Toxorhynchites) can be precluded from entering the housing due to their larger size than the exit slots. The one or more openings can be independently between about 2 mm and about 30 mm in at least one dimension, such as between about 3 mm and about 5 mm long and/or between about 3 mm and about 5 mm wide; between about 5 mm and about 10 mm long and/or between about 5 mm and about 10 mm wide; or between about 8 mm and about 12 mm long and between about 8 mm and about 12 mm wide. In some aspects, the one or more openings are large enough for adult insects such as mosquitoes to crawl and/or fly out while preventing other larger creatures including predators from crawling and/or flying into the closed housing and/or container. In some aspects, the one or more openings are of sufficient size for adult mosquitoes to crawl out but not fly out. The one or more openings can be independently of the same size or different sizes. In some aspects, the one or more openings can be independently in the shape of a triangle, circle, semi-circle, oval, ellipse, slot, square, rectangle, parallelogram, rhombus, trapezium, kite, or polygon (pentagon, hexagon, octagon, nonagon, decagon, etc.), or any combination thereof. The one or more openings can be independently of a regular or irregular shape, and can be of the same shape or different shapes. In some aspects, the one or more openings may be configured to prevent rain and/or a predator of the aquatic insect from entering the chamber. For instance, the one or more openings can be downward inclined from inside to outside of the housing.

Referring to FIG. 5, once the container 540 and the receptacle (not shown in FIG. 5) are placed inside the housing 520, the housing lid or cover 521 can be closed. For instance, the housing lid can engage a locking mechanism 527 provided on the housing and/or the container, such that the housing remains in a closed configuration until the locking mechanism is manipulated to release the lid. For instance, a spring mechanism 529 can pop open the lid once the housing is unlocked. In general, once the housing is locked after a fresh set of the container and the receptacle are placed inside the housing, there is no need to open the housing until the container and the receptacle need to be replaced, e.g., after all or the majority of adult insects having emerged from eggs in the receptacle and developed in water 570 are released into the target geographical area. Adult insects such as genetically engineered mosquitoes can exit the closed housing via one or more openings 523 connected to the inside of the closed housing and the chamber above the water, for instance, via one or more corresponding structures 525 (e.g., notches, through holes, channels, or the like) on the housing side wall and/or the container side wall. In some cases, however, the closed housing can be opened during egg hatching and/or during further development of the insects inside the housing (e.g., at the larva and/or pupa stages) and generally before adults emerge. In some cases, since egg hatching inside the housing can be synchronized, adults insects can emerge in a synchronized manner as well, such that the closed housing may be opened at one or more time points to allow the adults to escape, as an alternative (e.g., in cases where no exit slot for adult insects is provided) or in addition to allowing the adults to escape via exit slots. In some cases, adults insects can emerge in batches and the housing can be opened and closed multiple times. As such, FIG. 5 can depict the closing of the housing lid at a number of time points, including after inserting an initial set of container and receptacle (e.g., an egg/diet pot), after inserting a replacement set of container and receptacle, during insect development, and/or after one or more batches of adult insects have emerged in the housing.

Referring to FIG. 6A, the housing 620 in a closed configuration is shown and may or may not contain a container and/or a receptacle inside. The opening 623 (e.g., exit slot) is shown on housing lid 621, which may be opened by pressing an insert or bottom or the like 629 to release a releasable locking mechanism, for instance, as shown in FIG. 6B. The locking mechanism can comprise a quick release lid safety clip, e.g., 527 as shown in FIG. 5. As such, FIG. 6B can depict opening the housing lid at a number of time points, including before inserting an initial set of container and receptacle (e.g., an egg/diet pot), before inserting a replacement set of container and receptacle, during egg hatching, larva, and/or pupa stages, and/or before and/or after one or more batches of adult insects have emerged in the housing. The housing can be opened for a variety of purposes, for instance, for adding and/or removing water (e.g., without removing and/or harming larvae and/or pupae in the water), for adding one or more diet compositions and/or supplements, for adding one or more agents that regulate hatching and/or further development, for removing waste, for adding one or more water preservatives, for adjusting the amount and/or configuration of a thermal insulating material, for monitoring insect development, temperature (e.g., in the water and/or head space), water amount and/or quality, and/or nutrient level, or any combination thereof.

In some embodiments, the housing is configured to thermally insulate the interior of the housing, including various components of the apparatus disclosed herein, so as to maintain an internal temperature that facilitates rearing of insects including an aquatic insect. In some embodiments, the housing and/or the container comprises a temperature insulating material. In some embodiments, the apparatus, housing body, the lid, the bottom wall of the container, and/or the side wall of the container comprises a temperature insulating material. In some embodiments, the apparatus comprises a filler between the housing and the container when the container is placed inside the housing. In some embodiments, the filler comprises a temperature insulating material. In some embodiments, the filler comprises a foam. In some embodiments, the foam is a temperature insulating foam disposed on or contacting with an inner surface of the housing and/or an outer surface of the container. In some embodiments, the housing comprises a shell and a filler inside the shell. In some embodiments, the shell comprises polypropylene and the filler comprises polyurethane foam. In some embodiments, the housing is configured to maintain a temperature that facilitates rearing of insects including an aquatic insect in a container within the housing. Designs with or without insulation may be used. In some embodiments, the thickness of the housing can be designed and/or adjusted to facilitate maintaining a temperature inside the housing, for example, by providing insulation of the container from the outside environment. In some embodiments, the color of the exterior surface of the housing can be designed and/or adjusted to facilitate maintaining a temperature inside the housing. In some embodiments, the color of the exterior surface of the housing is configured to reduce the solar gain inside the housing. In some embodiments, the housing comprises cardboard. In some embodiments, the exterior surface of the housing can be white. In some embodiments, the housing can be placed in shaded locations in the field to maintain a temperature that facilitates rearing of insects including an aquatic insect.

In some embodiments, an apparatus or system disclosed herein may comprise additional features, such as one or more meshes, one or more fabric screens, and/or one or more cardboard or plastic panels, which may help further reduce solar heat gain to the apparatus, especially when the apparatus is deployed in a full sun location (such as posts or walls). In some embodiments, the housing can be used with one or more additional shields to further reduce the solar gain inside the housing. In some embodiments, the housing can be placed in locations with or without full sun and used with one or more additional shields (e.g., a mesh, a screen, a cardboard, or plastic panel) to further reduce the solar gain inside the housing. In some embodiments, the additional features, such as meshes or screens, can be built into the apparatus for deployment or recommended for use in specific applications when required. In some embodiments, one or more features may be added to the apparatus or system that offer thermal reduction as a result of physical means, which can include the addition of one or more solar panels, one or more fans, or one or more motors to control water or headspace parameters, or addition of a sealed chemical cooling sachet that is initiated and added to the housing and/or the container (e.g., attached to the outside surface of the container or a part that does not touch water) but not into the water. For instance, the solar panel may provide power to actuate the motor and/or the fan, and the motor can in turn actuate structures in the apparatus to change one or more water parameters and/or one or more headspace parameters. For example, the motor may actuate a lid (the “roof” of the headspace) such that when the lid is moved up or down or otherwise change position, shape, and/or dimension, the position, shape, and/or dimension of the headspace may also be changed. In some embodiments, the housing and/or additional insulation can provide an in-apparatus temperature suitable for rearing of insects including aquatic insects such as mosquitoes. In some embodiments, the exterior surface of the housing can be wrapped with an additional thermal insulation layer to maintain the in-apparatus temperature, for instance when the housing itself does not provide sufficient thermal insulation. In some embodiments, the apparatus is deployed in a location where the environmental temperature is suitable for rearing of insects including aquatic insects, even without thermal insulation provided by the housing and/or additional thermal insulating material. In some embodiments, the apparatus can be combined with additional physical means to maintain an in-apparatus temperature suitable for rearing of insects including aquatic insects such as mosquitoes. In some embodiments, the apparatus can be combined with using a fan. In some embodiments, the apparatus can be attached with one or more solar panels (e.g., to power one or more motors and/or the fans). In some embodiments, the apparatus can further include a sealed chemical cooling sachet wherein the sealed chemical cooling sachet can be initiated and added to the exterior and/or the interior space of the apparatus except for the water-containing container. The software and/or computer systems disclosed herein (e.g., as described in Section V) can be used to recommend the use of the one or more additional features, such as a mesh, a fabric screen, a cardboard, a plastic panel, a shield, a solar panel, a fan, a motor to control water or headspace parameters, and/or a sealed chemical cooling sachet.

In some embodiments, the apparatus, housing, and/or container are configured to have temperature control mechanisms. In some embodiments, the apparatus, housing, and/or container are configured for passive cooling. In some embodiments, the exterior of the housing comprises reflective coloring.

In some embodiments, the housing, the container, and/or the filler is configured to maintain an average temperature suitable for rearing the aquatic insect. In some embodiments, the housing, the container, and/or the filler is configured to maintain an average temperature of between about 10° C. and about 40° C. in the chamber and/or the hydrating composition therein. In some embodiments, the housing, the container, and/or the filler is configured to maintain an average temperature of between about 15° C. and about 32° C. in the chamber and/or the hydrating composition therein. In some embodiments, the housing, the container, and/or the filler is configured to maintain an average temperature of between about 20° C. and about 28° C. in the chamber and/or the hydrating composition therein. In any of the embodiments herein, the average temperature in the chamber and/or the hydrating composition therein can be about 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C. In any of the embodiments herein, the average temperature in the chamber and/or the hydrating composition therein may fluctuate as the outside temperature changes; however, the housing and/or the container are configured to keep the average temperature relatively stable compared to the outside temperature throughout the day, for days, and/or for weeks, such as at least three weeks or four weeks. In any of the embodiments herein, the average temperature in the chamber and/or the hydrating composition therein can be maintained between about 10° C. and about 40° C., between about 15° C. and about 32° C., or between about 20° C. and about 28° C., for about 7 days, 14 days, 21 days, 28 days, or longer.

In some embodiments, the container comprises a bottom wall and a side wall extending from the bottom wall, and wherein the bottom wall and the side wall are configured to form a chamber for holding a hydrating composition. In some embodiments, the container is configured to fit securely inside the housing body. In some embodiments, the container releasably and/or removably engages the housing body. In some embodiments, the container rests inside the housing body. In some embodiments, the container is configured for single use. In some embodiments, the container is replaceable. In some embodiments, the container is configured to be discarded or recycled. In some embodiments, the container comprises a recyclable material. In some embodiments, the container consists essentially of one or more recyclable materials. In some embodiments, the container comprises a recycled material, such as recycled PET (polyethylene terephthalate). In some embodiments, the container is configured for ease of shipping in multiples. In some embodiments, two or more of the containers can be stacked. In some embodiments, the container is placed in a housing configured to maintain the shape of the container. In some embodiments, the housing is configured to shield the container from the outside environment. In some embodiments, the housing comprises a folding such as a cardboard lip that can be folded to seal the device. In some embodiments, the housing comprises one or more escape holes that allow adult mosquitoes to exit the device. In some embodiments, the housing comprise cardboard coated with a layer which may prevent or delay water damage to the housing. In some embodiments, the housing is coated with wax. In some embodiments, the housing is coated with a resin, such as a water resistant resin material.

Referring to FIG. 1, the housing 120 and/or container 140 can comprise or be made of any suitable material or combination of materials. In general the housing and/or the container can comprise a temperature insulating material (also referred to as thermal insulating material). For instance, the housing body, the housing lid, the housing bottom wall, the housing side wall, the container bottom wall, and/or the container side wall can comprise or be made of a suitable temperature insulating material. In some particular aspects, a filler is provided between the housing and the container when the container is placed inside the housing. In other particular aspects, the housing and/or the container comprises a shell and a filler inside the shell. The filler can comprise or be made of a suitable temperature insulating material, optionally a foam (e.g., polyurethane foam, polyisocyanurate foam, or phenolic foam), mineral wool, cellular glass, plant material (e.g., straw, wood fiber, cotton, etc.), wool, cellulose, polystyrene (EPS), aerogel, or the like, or any combination thereof. The foam can be a temperature insulating foam disposed on or contacting with an inner surface of the housing and/or an outer surface of the container. In cases where the housing and/or the container comprises a shell, the temperature insulating foam can be inside the shell. For instance, the housing and the container can be a single piece comprising a shell, and the thermal insulating material can be provided inside the shell. The shell can comprise polypropylene and the filler can comprise a polyurethane foam. The housing, the container, and/or the filler can be configured to maintain an average temperature of between about 10° C. and about 40° C. in the chamber and/or the hydrating composition therein, such as between about 12° C. and about 35° C., between about 15° C. and about 32° C., between about 20° C. and about 28° C., or between about 22° C. and about 25° C. Without being bound by theory, the average temperature can be maintained (relative to the environment temperature which may fluctuate in a wider range) due to the shape and/or the size of the housing and the container, the material of the housing and the container (including the amount, shape, and/or configuration of the thermal insulating material), the size, shape, and/or configuration of the opening(s), the amount and/or thermal properties of the hydrating composition, or any combination thereof.

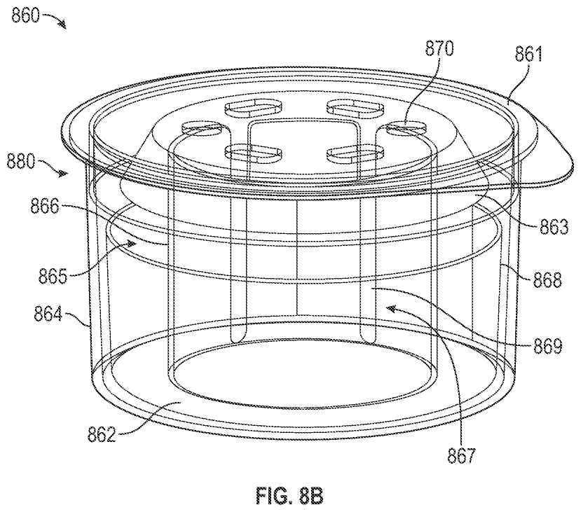

In general the housing and/or the container may comprise surfaces (e.g., inner surfaces of the housing lid or side wall in a closed housing or surfaces of the container above water) which adults of the aquatic insect may access. In some embodiments, the apparatus is configured to allow survival of an adult of the aquatic insect (e.g. an adult mosquito), for a period of time prior to release, and the interior of the housing and/or apparatus comprises a surface on which an adult of the aquatic insect is capable of landing and/or resting. For example, the inner surfaces of the housing and/or the container can provide a surface on which an adult of the aquatic insect can land and/or rest, e.g., by the tarsals of an adult of the aquatic insect gripping onto the surface or a structure thereon. In some embodiments, the inner surfaces of the housing and/or the container comprise a plurality of indentations and/or protrusions. Referring to FIG. 7, the surfaces (e.g., surfaces of container 740 above water fill line 772) are grippable by tarsals of the adults (e.g., mosquitoes) such that the insects do not exhaust themselves while trying to escape from the closed housing. In some aspects, the inner surfaces of a closed housing that are accessible to adults comprise a plurality of indentations, protrusions, and/or other structures for resting. The size, shape, and/or configuration (e.g., the angle of a resting surface) of the indentations, protrusion, and/or other structures can be optimized for the particular species. For instance, Anopheles adults rest at an angle of about 45 degrees to the surface they are standing on, while adult Aedes and Culex rest with the body parallel to the surface.

In some embodiments, the container comprises one or more adapter structures configured to engage various components. In some embodiments, the container comprises one or more adapter structures configured to engage the receptacle. In some embodiments, the container comprises one or more adapter structures configured to engage a water preservative or a structure comprising and/or containing a water preservative (e.g., a packaged or encapsulated water preservative), and/or engage a structure configured to comprise and/or contain a water preservative (e.g., a capsule, or a container that can be filled with a water preservative and optionally with an inert filler substance). In some embodiments, the water preservative may be packaged or encapsulated together with an inert filler substance (e.g., a salt), a binder, a disintegrant, a lubricant, a coloring agent, one or more other preservatives, or any combination thereof. In some embodiments, the one or more adapter structures are on the container bottom wall and/or side wall.

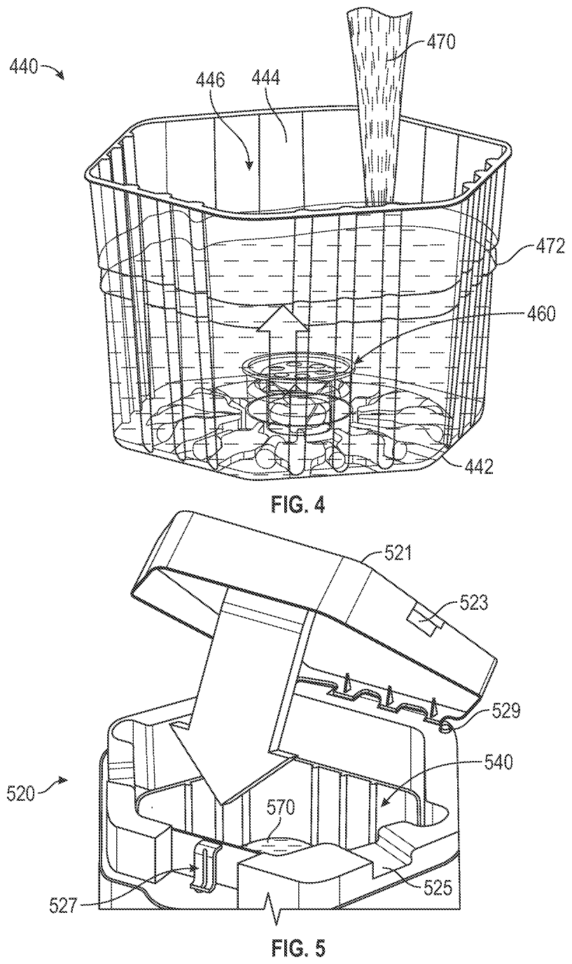

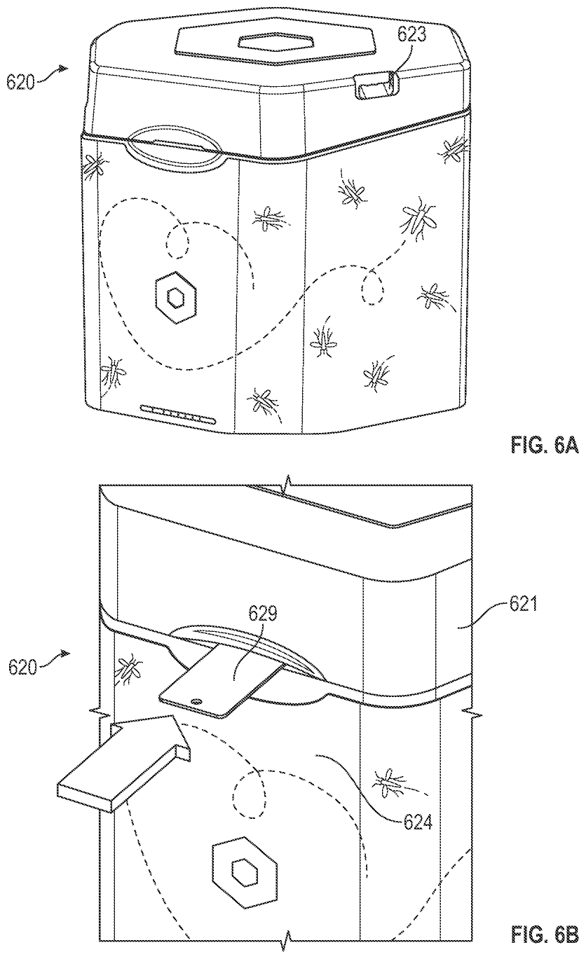

Referring to FIG. 3, the receptacle 360 is configured to engage one or more adapter structures 348 in the container 340. The container can comprises one or more additional adapter structures 350 configured to engage one or more additional components 352 (e.g., water preservative capsules) other than the receptacle. In general the adapter structures 348 and 350 can be on the bottom wall 342 of the container (or the housing in cases where no separate container is used), but the locations of the adapter structures are not so limited as long as the receptacle and/or the one or more additional components can contact the hydrating composition when it is added to the chamber 346 formed in the container or in the housing. For instance, the adapter structures 348 and 350 can be provided on the side wall 344 such that at least a portion of the receptacle and/or at least a portion of the one or more additional components (e.g., water preservative capsules) are below a water fill line (e.g., 472 as shown in FIG. 4).

One or more additional components may be added in the container, including one or more water preservatives. As shown in FIG. 3, the water preservative can be provided in one or more capsules 352 (e.g., containing water preservative powders in a dissolvable shell) clipped into an adapter structure 350 on the bottom wall 342. As an alternative or in addition to the capsule(s), water preservative(s) can be added as a powder, tablets or pellets pre-loaded (e.g., packed) in the container, and/or as a separate additive dissolved and/or dispersed into the hydrating composition by the user. In some aspects, water preservative(s) can be pre-loaded in the receptacle, e.g., in the same section as the diet and/or the eggs or in a separate section, as long as the water preservative(s) can be dissolved and/or dispersed into the hydrating composition once it is added. In some embodiments, water preservative(s) can be provided and/or stored in sachets and can be sprinkled into the container and/or the hydrating composition by a user. Any suitable water preservative can be used and may comprise sorbic acid or a salt or solvate thereof, a chlorinated hydroxytriazine or a salt or solvate thereof, and/or citric acid or a salt or solvate thereof. In some embodiments, the water preservative may comprise a sorbic acid or a salt, solvate, or derivative (e.g., an ester) thereof; a dichloroisocyanuric acid or a salt, solvate, or derivative (e.g., an ester) thereof; a metabisulphite or a salt, solvate, or derivative thereof; bronopol or a derivative thereof; a benzoic acid or a salt, solvate, or derivative (e.g., an ester) thereof; a formic acid or a salt, solvate, or derivative (e.g., an ester) thereof; a chloric acid or a salt, solvate, or derivative (e.g., an ester) thereof; a nitrous acid or a salt, solvate, or derivative (e.g., an ester) thereof; a propionic acid or a salt, solvate, or derivative (e.g., an ester) thereof; and/or a citric acid or a salt, solvate, or derivative (e.g., an ester) thereof. For instance, suitable water preservative may comprise potassium sorbate, sodium dichloroisocyanurate, sodium metabisulphite, bronopol, sodium benzoate, sodium formate, sodium chloride, sodium nitrite, sodium propionate, and/or citric acid. In some embodiments, the water preservative(s) can be provided in a suitable form, such as a solid, liquid, semi-solid, gel, syrup, paste, powder, agglomerate, granule, compressed material, reconstitutable solid, suspension, lump, particulate, bead, emulsion, or wet lump, or any combination or mixture thereof. In some embodiments, the water preservative(s) can be provided in sachets, capsules, pellets, and/or tablets which further comprises a filler substance to facilitate the accurate dispensing of the water preservative and the formation of the sachets, capsules, pellets, and/or tablets. Any suitable filler substance can be used and may comprise cellulose or calcium phosphate, or any combination thereof. In some embodiments, the filler substance may comprise cellulose. In some embodiments, the filler substance may comprise calcium phosphate. In some embodiments, the water preservative(s) can be provided in sachets, capsules, pellets, and/or tablets which further comprises an effervescent reagent to promote the distribution of the water preservative in the hydrating compositions. In some embodiments, the effervescent reagent may comprise sodium bicarbonate or solvate thereof. In some embodiments, the effervescent reagent may comprise adipic acid or a salt or solvate thereof. In some embodiments, the effervescent reagent may comprise citric acid anhydrous or a salt or solvate thereof. In some embodiments, the effervescent reagent may be provided in a suitable form, such as a solid, liquid, semi-solid, gel, syrup, paste, powder, agglomerate, granule, compressed material, reconstitutable solid, suspension, lump, particulate, bead, emulsion, or wet lump, or any combination or mixture thereof. In some embodiments, the sachets, capsules, pellet, and/or tablets may comprise sodium dichloroisocyanurate, citric acid, sodium bicarbonate and/or cellulose. In some embodiments, the sachets, capsules, pellet, and/or tablets may comprise potassium sorbate, citric acid, and/or sodium bicarbonate.

Referring to FIG. 4, the container and/or housing 440 may comprise a volume indicator of the hydrating composition 470 in the chamber 446. The volume indicator may comprise a fill line 472 on the side wall 444 of the container and/or housing. In general the apparatus is configured to provide a head space between the hydrating composition and the housing in a closed configuration, when the container is placed inside the housing and the hydrating composition is in the chamber. The hydrating composition can be water, and the hydrating composition in the chamber can be in any suitable volume, e.g., about 200 mL, about 300 mL, about 400 mL, about 500 mL, about 600 mL, about 700 mL, about 800 mL, about 900 mL, about 1,000 mL, about 1,100 mL, about 1,200 mL, about 1,300 mL, about 1,400 mL, about 1,500 mL, about 1,600 mL, about 1,700 mL, about 1,800 mL, about 1,900 mL, about 2,000 mL, about 2,500 mL, about 3,000 mL, about 3,500 mL, about 4,000 mL, about 4,500 mL, about 5,000 mL, about 5,500 mL, about 6,000 mL, about 6,500 mL, about 7,000 mL, about 7,500 mL, about 8,000 mL, about 8,500 mL, about 9,000 mL, about 9,500 mL, about 10,000 mL, or more. The receptacle 460 can be partially or fully submerged in the hydrating composition 470. For instance, the receptacle can be securely affixed to the bottom wall 442 and the top of the receptacle remains below the fill line 472 such that the hydrating composition 470 can keep the eggs and at least a portion of the diet wet during egg hatching and further development of the insects until adulthood. The receptacle can be clipped into an adapter structure on the bottom wall, tied to the bottom wall, and/or held to the bottom wall by a force from above the receptacle.

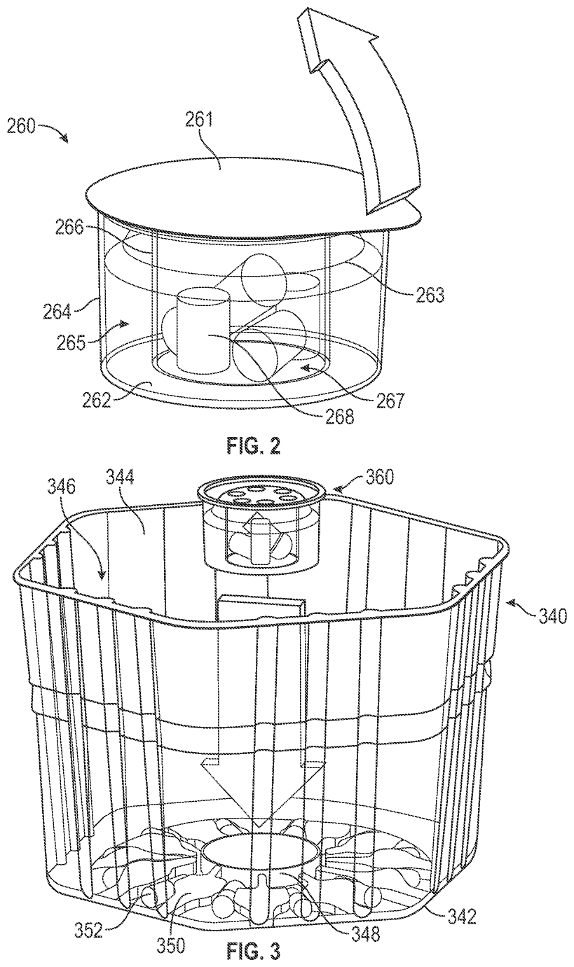

In some aspects, an apparatus disclosed herein comprises a manufactured, sealable receptacle that can be attached to the container and is capable of holding the eggs of an aquatic insect and optionally the diet for the insect, both of which become at least partially wet in either order when the hydrating composition (e.g., a water-based solution) is added. Referring to FIG. 2, the receptacle 260 comprises a receptacle base comprising a receptacle bottom wall 262, a receptacle side wall 264, and one or more inner wall structures 266, such as an enclosure enclosing a diet section 267 configured to house one or more diet formulations 268 such as diet pellets or any other suitable form, such as a solid, liquid, semi-solid, gel, syrup, paste, powder, agglomerate, granule, compressed material, reconstitutable solid, suspension, lump, particulate, bead, emulsion, or wet lump, or any combination or mixture thereof.

The receptacle side wall and/or the one or more inner wall structures can form an egg section 265 within the receptacle base, where the egg section and the diet section are connected, e.g., capable of forming a fluid communication between the two sections. Once the hydrating composition such as water enters the receptacle, it can flow from the diet section to the egg section, or vice versa. The receptacle 260 can further comprise a receptacle lid 263 comprising a plurality of perforations (e.g., 870 as shown in FIG. 8A) and configured to engage the receptacle base. In some aspects, the receptacle 260 can further comprise a receptacle seal 261. The receptacle seal can be configured to form an airtight seal with the receptacle base and/or the receptacle lid, thereby sealing the egg section and the diet section from an outside environment. In some aspects, the sealed receptacle and contents therein (e.g., one or more diet formulations) maintain the freshness and viability of the eggs in the sealed receptacle. The seal can be removed (e.g., immediately prior to use) in order for water to wet the eggs (for hatching) and the diet (e.g., for providing nutrients to the insects throughout the larval stage). In some embodiments, the diet may optionally provide nutrients to the insects throughout the larval, pupal, and/or adult stages, e.g., until the adults exit. In some aspects, the seal can disintegrate in the presence of water, e.g., the seal can be a dissolvable seal. The receptacle can in a variety of configurations, e.g., as shown in FIGS. 8A-8B and FIGS. 9A-9B.

Various non-limiting embodiments of the receptacle are described in Section II below for the purpose of illustration.

### II. RECEPTACLE AND COMPONENTS THEREOF

In addition to the housing and/or the container, the apparatus comprises one or more receptacles, such as one, two, three, or more receptacles, that are configured to hold eggs of the aquatic insect and to engage the housing and/or the container.

In some embodiments, the one or more receptacles herein comprise a sealable enclosure comprising eggs of an arthropod and/or diet for the arthropod; and a removable seal forming an airtight seal separating the enclosure from an outside environment. For instance, when the seal is removed and at least a portion of the receptacle contacts a hydrating composition, the hydrating composition enters the sealable enclosure.

In some embodiments, the one or more receptacles herein comprise a sealable enclosure comprising an egg section and a diet section, the egg section and the diet section separated by a barrier with one or more openings; and optionally a removable seal forming an airtight seal separating the enclosure from an outside environment. In some aspects, when the seal is removed and at least a portion of the receptacle contacts a hydrating composition, the hydrating composition enters the egg section and the diet section.

In some embodiments, the one or more receptacles herein comprise a sealable enclosure comprising an egg section comprising eggs of an arthropod and a diet section comprising diet for the arthropod, where the egg section and the diet section are separated by a barrier with one or more openings. The receptacle may further comprise a removable seal forming an airtight seal separating the enclosure from an outside environment. For instance, when the seal is removed and at least a portion of the receptacle contacts a hydrating composition, the hydrating composition can enter the egg section and the diet section to wet at least a portion of the eggs and at least a portion of the diet.

In some aspects, the one or more receptacles are additionally configured to hold a diet composition for insect larvae and/or an agent that regulates insect development (e.g., a hatch stimulant such as cysteine and/or ascorbic acid). In other aspects, the diet composition and/or the development regulating agent may be provided separately from the one or more receptacles, for instance, by adding the diet and/or agent into the hydrating composition (and outside the receptacle) before, during, or after inserting a receptacle in the container.

Referring to FIG. 1, the receptacle 160 can be of any suitable design such that when the hydrating composition is in chamber 146 (provided in container 140 or an integrated housing/container), at least a portion of receptacle 160 contacts the hydrating composition to establish a fluid communication between an inner cavity of the receptacle and the hydrating composition. In cases where receptacle 160 is inserted in the container and affixed to the bottom wall, addition of water to chamber 146 can cause water to contact (e.g., submerge) the receptacle. A seal of the receptacle may be removed prior to or after contacting water, and water may enter the receptacle to wet the eggs (and optionally at least a portion of another component such as diet and/or an egg hatch stimulant). In other cases, water can be added into chamber 146 before or after the container is placed in the housing, and then receptacle 160 is inserted in the container and affixed to the bottom wall, optionally followed by adding more water to the chamber to a fill line.

In some embodiments, the apparatus comprises a receptacle. In some embodiments, the receptacle comprises: (a) a receptacle base comprising: (i) a receptacle bottom wall, (ii) a receptacle side wall extending from the receptacle bottom wall, and (iii) an enclosure, optionally extending from the receptacle bottom wall, wherein the receptacle side wall and/or the enclosure forms an egg section and a diet section within the receptacle base, and wherein the egg section and the diet section are connected (e.g., via air communication); (b) a receptacle lid comprising a plurality of perforations and configured to engage the receptacle base; and (c) a receptacle seal configured to form an airtight seal with the receptacle base and/or the receptacle lid, thereby sealing the egg section and the diet section from an outside environment.

In some embodiments, the receptacle comprises: (a) a sealable enclosure comprising an egg section comprising eggs of an aquatic insect (and optionally a hatch stimulant), and a diet section comprising diet for the aquatic insect, optionally wherein the egg section and the diet section are separated by a barrier with one or more openings; and (b) a removable seal forming an airtight seal separating the enclosure from an outside environment; wherein when the seal is removed and at least a portion of the receptacle contacts a hydrating composition, the hydrating composition enters the egg section and the diet section and contacts at least a portion of the eggs and at least a portion of the diet.

In some embodiments, the receptacle is configured to hold eggs of an aquatic insect in the egg section. In some embodiments, the receptacle is configured to hold between about 0.01 g and about 0.5 g, between about 0.01 g and about 0.02 g, between about 0.02 g and about 0.03 g, between about 0.03 g and about 0.04 g, between about 0.04 g and about 0.05 g, between about 0.05 g and about 0.06 g, between about 0.06 g and about 0.07 g, between about 0.07 g and about 0.08 g, between about 0.08 g and about 0.09 g, between about 0.09 g and about 0.1 g, between about 0.1 g and about 0.15 g, between about 0.15 g and about 0.2 g, between about 0.2 g and about 0.25 g, between about 0.25 g and about 0.3 g, between about 0.3 g and about 0.35 g, between about 0.35 g and about 0.4 g, between about 0.4 g and about 0.45 g, or between about 0.45 g and about 0.5 g eggs. In some embodiments, the receptacle comprises an adhesive surface on the receptacle side wall, on the receptacle bottom wall, on the enclosure, and/or in the egg section, configured to adhere to eggs of the aquatic insect. In some embodiments, the receptacle is configured to hold diet for an aquatic insect in the diet section, optionally wherein the diet is formulated to dissolve and/or disintegrate in a controlled manner when contacted with water. In some embodiments, the egg section and/or the diet section is configured to hold a hatch stimulant. In some embodiments, the hatch stimulant is cysteine or ascorbic acid or a salt or solvate thereof. In some embodiments, the egg section and the diet section are separated by the enclosure which is perforated and/or castellated. In some embodiments, the enclosure comprises a plurality of panels extending from the receptacle bottom wall. In some embodiments, the enclosure and the receptacle side wall form concentric circles. In some embodiments, one or more of the plurality of perforations on the receptacle lid are configured to permit passage of water and a larva of an aquatic insect. In some embodiments, one or more of the plurality of perforations on the receptacle lid are independently between about 5 mm and about 50 mm in at least one dimension. In some embodiments, the receptacle seal forms an airtight seal with the receptacle side wall, and the receptacle lid is inside the receptacle base.

Referring to FIG. 8A, provided herein is a receptacle 860 comprising an egg section 865 for holding eggs (not shown) of the aquatic insect. The receptacle can comprise a receptacle lid 863 and a receptacle base 880 that engages the lid to form an internal cavity. The receptacle can be configured to provide a fluid communication between the internal cavity (e.g., the egg section and/or the diet section) and the hydrating composition, for instance, via one or more perforations 870 on the receptacle lid 863. The one or more perforations can be of any suitable size, shape, and/or configuration. In addition to the perforation size, shape, and/or configuration, the total number of perforations and their locations on the receptacle lid can also be selected to optimize wetting of the eggs and/or diet. In some embodiments, mosquito eggs are provided on an surface comprising an adhesive (to which the eggs adhere), such as an adhesive tape or strip, and the eggs do not stick to the walls of the receptacle. In other embodiments, the eggs can be provided on the receptacle bottom wall 862, the receptacle side wall 864, the outside wall of an enclosure 866, and/or an insert 868 (e.g., a tape or strip which is optionally adhesive on one or both surfaces). The insert is optional since an adhesive can be sprayed and/or painted directly onto a surface in or enclosing the egg section. For instance, an adhesive can be sprayed and/or painted on at least a portion of the receptacle bottom wall 862, at least a portion of the receptacle side wall 864, and/or at least a portion of the outside wall of the enclosure 866. The enclosure, optionally with at least a portion of the receptacle side wall and/or at least a portion of the receptacle bottom wall, can form a diet section 867 for housing one or more diet compositions. The enclosure and the diet section formed thereby are optional, since the one or more diet compositions and the eggs can be provided in the same section (e.g., a chamber or cavity inside the assembled receptacle). In some aspects, the diet and the eggs can be pre-mixed, while in other aspects the diet and the eggs can be kept separate (e.g., in different chambers and/or cavities) until wetting by water brings the eggs closer to the diet or vice versa. In particular aspects, the enclosure is perforated or castellated such as comprising one or more slits 869, thereby permitting water to move between the egg section and the diet section. In some aspects, water can enter the receptacle through perforations 870, wetting the eggs and/or the diet in any suitable order. In some aspects, water enters through perforations 870 into the egg section 865 and the diet section 867 at the same time. In some aspects, the receptacle lid and perforations thereon are such that water first wets the diet in the diet section enclosed by enclosure 866, and then enters egg section 865 to wet the eggs therein through slits 869. Hatching can occur in the receptacle, e.g., in the egg section; however, once larvae emerge they can move between the egg section and the diet section, and/or between water in the receptacle and water outside the receptacle. For example, larvae can exit perforations 870 into the chamber in the container.

Prior to use, the receptacle base can be sealed by a seal 861 to form a sealed receptacle 860 as shown in FIG. 8B.

In some embodiments, the apparatus comprises a receptacle. In some embodiments, the receptacle comprises: (a) a receptacle base comprising (i) a receptacle bottom wall, and (ii) a receptacle side wall extending from the receptacle bottom wall; (b) an enclosure comprising a tubular body and a flange disposed around the tubular body between a bottom end and an upper end of the tubular body, wherein the tubular body comprises a bottom opening, wherein the flange comprises a plurality of perforations, wherein the flange engages the receptacle side wall, the receptacle side wall and/or the tubular body forms an egg section and a diet section within the receptacle base, the egg section and the diet section are connected, and the egg section is connected to a space above the flange via the plurality of perforations on the flange; (c) a receptacle seal configured to form an airtight seal with the receptacle base, thereby sealing the enclosure, the egg section, and the diet section from an outside environment.

In some embodiments the enclosure is configured to insert into the receptacle base. In some embodiments, the enclosure is disposed on or integral to the receptacle bottom wall. In some embodiments, the upper end to the tubular body is closed. In some embodiments, the tubular body further comprises an upper opening and a closer reversibly or irreversibly closing the upper opening. In some embodiments, the closer does not comprise a perforation.

In some embodiments, the receptacle is configured to hold eggs of an aquatic insect in the egg section. In some embodiments, the receptacle is configured to hold between about 0.01 g and about 0.5 g, between about 0.01 g and about 0.02 g, between about 0.02 g and about 0.03 g, between about 0.03 g and about 0.04 g, between about 0.04 g and about 0.05 g, between about 0.05 g and about 0.06 g, between about 0.06 g and about 0.07 g, between about 0.07 g and about 0.08 g, between about 0.08 g and about 0.09 g, between about 0.09 g and about 0.1 g, between about 0.1 g and about 0.15 g, between about 0.15 g and about 0.2 g, between about 0.2 g and about 0.25 g, between about 0.25 g and about 0.3 g, between about 0.3 g and about 0.35 g, between about 0.35 g and about 0.4 g, between about 0.4 g and about 0.45 g, or between about 0.45 g and about 0.5 g eggs. In some embodiments, the receptacle comprises an adhesive surface on the receptacle side wall, on the receptacle bottom wall, on the tubular body, and/or in the egg section, configured to adhere to eggs of the aquatic insect.

In some embodiments, the receptacle is configured to hold diet for an aquatic insect in the diet section, optionally wherein the diet is formulated to dissolve and/or disintegrate in a controlled manner, e.g., at a controlled rate, when contacted with the hydrating composition such as water. In some embodiments, the release of nutrients from the diet in water is minimized in the first about three, about four, or about five days after contacting water. In some embodiments, at least about 50%, at least about 60%, at least about 70%, at least about 80%, at least about 90%, or at least about 95% of the diet is not dissolved and/or disintegrated within the first two, three, four, five, or six days of contacting the water.

In some embodiments, the egg section and/or the diet section is configured to hold a hatch stimulant. In some embodiments, the hatch stimulant comprises cysteine, ascorbic acid, or a salt or solvate thereof.

In some embodiments, the egg section and the diet section are connected via the bottom opening of the tubular body, optionally wherein a bottom section of the tubular body is perforated and/or castellated. In some embodiments, the tubular body and the receptacle side wall form concentric circles. In some embodiments one or more of the plurality of perforations on the flange are configured to permit passage of water and a larva of an aquatic insect. In some embodiments, one or more of the plurality of perforations on the flange are independently between about 1 mm and about 50 mm in at least one dimension. In some embodiments, the plurality of perforations are at least about 1 mm, at least about 2 mm, at least about 3 mm, at least about 4 mm, at least about 5 mm, at least about 6 mm, at least about 7 mm, at least about 8 mm, at least about 9 mm, or at least about 10 mm in at least one dimension.

In some embodiments, the receptacle seal forms an airtight seal with the receptacle side wall, and the receptacle insert is inside the receptacle base. In some embodiments, the receptacle seal forms an airtight seal with the receptacle insert, which in turn forms an airtight seal with the receptacle base.

Referring to FIG. 9A, provided herein is an alternative design where receptacle 960 comprises an egg section 965 for holding eggs (not shown) of the aquatic insect. The receptacle can comprise a receptacle lid 963 that engages an enclosure 966. The enclosure can comprise a tubular body 972 and a flange 974 disposed around the tubular body between a bottom end and an upper end of the tubular body. The tubular body can comprise a bottom opening 973. The flange 974 engages the receptacle side wall 964, which together with the tubular body forms the egg section 965 within the receptacle base 980. A diet section 967 can be formed by the receptacle lid 963 and the enclosure 966. The egg section and the diet section is connected, and the egg section is connected to a space 971 above the flange via one or more perforations 970 on the flange 974. The receptacle can be configured to provide a fluid communication between the egg section and/or the diet section and the hydrating composition, for instance, via one or more perforations 970. The one or more perforations can be of any suitable size, shape, and/or configuration. In addition to the perforation size, shape, and/or configuration, the total number of perforations and their locations can also be selected to optimize wetting of the eggs and/or diet. The eggs can be provided on the receptacle bottom wall 962, the receptacle side wall 964, the outside wall of the enclosure 966, and/or an insert 968 (e.g., a tape or strip which is optionally adhesive on one or both surfaces). The insert is optional since an adhesive can be sprayed and/or painted directly onto a surface in or enclosing the egg section. For instance, an adhesive can be sprayed and/or painted on at least a portion of the receptacle bottom wall 962, at least a portion of the receptacle side wall 964, and/or at least a portion of the outside wall of the enclosure 966. The enclosure, together with at least a portion of the receptacle bottom wall, can form a diet section 967 for housing one or more diet compositions. The enclosure and the diet section formed thereby are optional, since the one or more diet compositions and the eggs can be provided in the same section (e.g., a chamber or cavity inside the assembled receptacle). In some aspects, the diet and the eggs can be pre-mixed, while in other aspects the diet and the eggs can be kept separate (e.g., in different chambers and/or cavities) until wetting by water brings the eggs closer to the diet or vice versa. In particular aspects, the enclosure is perforated or castellated at the bottom opening 973, thereby permitting water to move between the egg section and the diet section. In some aspects, water can enter the receptacle through perforations 970, wetting the eggs and/or the diet in any suitable order. In some aspects, water enters through perforations 870 into the egg section 865 and the diet section 867 at the same time. In some aspects, the flange and perforations thereon are such that water first wets the eggs in the egg section, and then enters the diet section via the perforated or castellated bottom opening to wet the diet near the bottom opening. In some aspects, when the eggs are wetted, a portion of the diet in the diet section above the bottom opening is kept dry. As the wetted diet dissolves and/or disintegrates into the hydrating composition (e.g., water) and/or becomes consumed by larvae of the aquatic insect, dry diet above the wetted diet can be lowered to be wetted, thereby providing diet in a controlled manner over a period of time. Hatching can occur in the receptacle, e.g., in the egg section; however, once larvae emerge they can move between the egg section and the diet section, and/or between water in the receptacle and water outside the receptacle. For instance, larvae can exit perforations 970 into the chamber in the container.

Prior to use, the receptacle base can be sealed by a seal 961 to form a sealed receptacle 960 as shown in FIG. 9B.

### A. Egg Section and Diet Section

In some embodiments, the receptacle is configured to hold eggs of the aquatic insect and engage the container such that when the hydrating composition is in the chamber, at least a portion of the receptacle contacts the hydrating composition to establish a fluid communication between an inner cavity of the receptacle and the hydrating composition. In some embodiments, the receptacle is partially or fully submerged in the hydrating composition. In some embodiments, the receptacle is coupled (e.g., attached, fastened, clipped, secured, held) to the container or an adapter structure thereof. In some embodiments, the receptacle is coupled to an adapter structure on the bottom wall or side wall, and/or held to the bottom wall by a force from above the receptacle. In some embodiments, the receptacle is coupled to the interior of the housing. For instance, the receptacle can be fastened to a roof (e.g., the inner side of the housing lid) and be partially submerged in water when the lid is closed.

In some embodiments, the receptacle is configured to hold eggs of the aquatic insect. In some embodiments, the receptacle is configured to hold diet for of the aquatic insect. In some embodiments, the inner cavity of the receptacle comprises an egg section for holding eggs of the aquatic insect. In some embodiments, the inner cavity of the receptacle comprises a diet section for holding diet for the aquatic insect. In some embodiments, the receptacle is configured to provide a fluid communication between the egg section and the hydrating composition when at least a portion of the receptacle contacts the hydrating composition.

In some embodiments, prior to contacting the hydrating composition, the receptacle is a sealed receptacle. In some embodiments, the sealed receptacle is configured to preserve the viability of the aquatic insect eggs. In some embodiments, the sealed receptacle is configured to preserve the environment of the inner cavity, including humidity. In some embodiments, the sealed receptacle maintains an internal relative humidity that maintains viability of the eggs of the aquatic insect. In some embodiments, the sealed receptacle maintains an internal relative humidity of between about 55% and 75%. In some embodiments, the internal relative humidity of the sealed receptacle is or is maintained between about 55% and about 60%, between about 60% and about 65%, between about 65% and about 70%, between about 70% and about 75%, between about 75% and about 80%, or between about 85% and about 90%.

In some embodiments, the receptacle is a sealed receptacle comprising a removable seal. In some embodiments, the receptacle comprises a seal configured to form an airtight seal between the inner cavity and an outside environment. In some embodiments, the seal seals the egg section and the diet section from the outside environment. In some embodiments, the seal is removable. In some embodiments, the seal comprises foil and is manually removable. In some embodiments, the seal is manually removed prior to the receptacle contacting the hydrating composition. In some embodiments, the seal is dissolvable in the hydrating composition.

In some embodiments, the receptacle comprises an internal surface configured to hold or attach to the eggs of the aquatic insect. In some embodiments, the internal surface is comprised in the egg section. In some embodiments, the internal surface comprises an adhesive. In some embodiments an adhesive substance is applied, sprayed, or painted onto the internal surface. In some embodiments, the internal surface is comprised on an adhesive strip. In some embodiments, the adhesive strip is a one-sided or two-sided adhesive strip. In some embodiments, the adhesive strip is affixed to a surface enclosing the egg section. In some embodiments, the adhesive is an acrylic adhesive.

In some embodiments, the apparatus comprises a hatch stimulant. In some embodiments, the stimulant stimulates synchronous larval hatch from the eggs after contacting the hydrating composition. In some embodiments, the hatch stimulant is in the receptacle, and/or in close proximity to the eggs. In some embodiments, the hatch stimulant is adhered to the internal surface of the receptacle which the eggs are adhered to. In some embodiments, the hatch stimulant reduces oxygen potential sufficient to trigger a generally synchronous larval hatch from the eggs. In some embodiments, the hatch stimulant is dissolvable in water. In some embodiments, the amount and/or concentration of stimulant is sufficient to stimulate synchronous larval hatch. In some embodiments, the amount and/or concentration of stimulant is not sufficient to kill or negatively affect the viability of the eggs and/or larvae of the aquatic insect. In some embodiments, the hatch stimulant comprises cysteine, ascorbic acid, or a salt or solvate thereof, or any combination thereof.

In some embodiments, the receptacle comprises a diet section for holding diet for the aquatic insect larvae. In some embodiments, the receptacle is configured to provide fluid communication between the diet section and the hydrating composition when at least a portion of the receptacle contacts the hydrating composition. In some embodiments, the diet section is configured to allow the hydrating composition to wet all, or at least a portion of, the diet in the diet section. In some embodiments, the diet section is configured to allow the hydrating composition to wet a portion of the diet while maintaining a portion of the diet dry. In some embodiments, the diet section is configured to allow the hydrating composition to wet the diet in a bottom section of the diet section while maintaining dry diet in a top section of the diet section. In some embodiments, the diet and/or diet section is separated from the eggs and/or egg section by an enclosure or barrier with one or more openings that allow fluid communication. In some embodiments, the diet and/or diet section is separated from the eggs and/or egg section by a perforated, slotted, or castellated enclosure or barrier.

### B. Eggs

In some embodiments, the sealed receptacle contains between about 0.01 g and about 0.5 g, between about 0.01 g and about 0.02 g, between about 0.02 g and about 0.03 g, between about 0.03 g and about 0.04 g, between about 0.04 g and about 0.05 g, between about 0.05 g and about 0.06 g, between about 0.06 g and about 0.07 g, between about 0.07 g and about 0.08 g, between about 0.08 g and about 0.09 g, between about 0.09 g and about 0.1 g, between about 0.1 g and about 0.15 g, between about 0.15 g and about 0.2 g, between about 0.2 g and about 0.25 g, between about 0.25 g and about 0.3 g, between about 0.3 g and about 0.35 g, between about 0.35 g and about 0.4 g, between about 0.4 g and about 0.45 g, or between about 0.45 g and about 0.5 g eggs of the aquatic insect.

In some embodiments, at least about 10%, at least about 20%, at least about 30%, at least about 40%, at least about 50%, at least about 60%, at least about 70%, at least about 75%, at least about 80%, at least about 85%, at least about 90%, at least about 95%, or at least about 99% of the eggs in the sealed receptacle are viable and/or hatch. In some embodiments, at least about 10%, at least about 20%, at least about 30%, at least about 40%, at least about 50%, at least about 60%, at least about 70%, at least about 75%, at least about 80%, at least about 85%, at least about 90%, at least about 95%, or at least about 99% of the male eggs in the sealed receptacle are viable and/or hatch. In some embodiments, a majority of the eggs (male and female) in the sealed receptacle are viable and/or hatch.

In some embodiments, the receptacle comprises male and female eggs. In some embodiments, the receptacle comprises male eggs only. In some embodiments, the receptacle comprises female eggs only. As such, the eggs may but do not need to be sex sorted. In some embodiments, the eggs are approximately 50% male and 50% female. In some embodiments, a majority of the eggs are male. In some embodiments, at least about 10%, at least about 20%, at least about 30%, at least about 40%, at least about 50%, at least about 60%, at least about 70%, at least about 75%, at least about 80%, at least about 85%, at least about 90%, at least about 95%, at least about 99%, or 100% of the eggs are male.

In some embodiments, the apparatus is configured to contain and/or yield between about 1,000 and about 50,000 larvae of the aquatic insect, optionally wherein the apparatus contains between about 1,000 and about 2,000, between about 2,000 and about 3,000, between about 3,000 and about 4,000, between about 4,000 and about 5,000, between about 5,000 and about 6,000, between about 6,000 and about 7,000, between about 7,000 and about 8,000, between about 8,000 and about 9,000, between about 9,000 and about 10,000, between about 10,000 and about 15,000, between about 15,000 and about 20,000, between about 20,000 and about 25,000, between about 25,000 and about 30,000, between about 30,000 and about 35,000, between about 35,000 and about 40,000, between about 40,000 and about 45,000, or between about 45,000 and about 50,000 larvae of the aquatic insect.

In some embodiments, the apparatus is configured to contain and/or yield between about 500 and about 25,000 hatched male larvae of the aquatic insect, optionally wherein the apparatus contains between about 500 and about 1,000, between about 1,000 and about 1,500, between about 1,500 and about 2,000, between about 2,000 and about 3,000, between about 3,000 and about 4,000, between about 4,000 and about 5,000, between about 5,000 and about 6,000, between about 6,000 and about 7,000, between about 7,000 and about 8,000, between about 8,000 and about 9,000, between about 9,000 and about 10,000, between about 10,000 and about 15,000, between about 15,000 and about 20,000, or between about 20,000 and about 25,000 male larvae of the aquatic insect.

In some embodiments, the larvae comprise males and females. In some embodiments, the larvae are approximately 50% male and 50% female. In some embodiments, a majority of the larvae are male. In some embodiments, at least about 10%, at least about 20%, at least about 30%, at least about 40%, at least about 50%, at least about 60%, at least about 70%, at least about 75%, at least about 80%, at least about 85%, at least about 90%, at least about 95%, at least about 99%, or 100% of the larvae are male.

In some embodiments, the apparatus is configured to yield between about 250 and about 500, between about 500 and about 1,000, between about 1,000 and about 1,200, between about 1,200 and about 1,400, between about 1,400 and about 1,600, between about 1,600 and about 1,800, between about 1,800 and about 2,000, between about 2,000 and about 3,000, between about 3,000 and about 4,000, between about 4,000 and about 5,000, between about 5,000 and about 6,000, between about 6,000 and about 7,000, between about 7,000 and about 8,000, between about 8,000 and about 9,000, or between about 9,000 and about 10,000 adults of the aquatic insect.

In some embodiments, the adults of the aquatic insect that the apparatus yields comprise males and females. In some embodiments, the adults of the aquatic insect that the apparatus yields are approximately 50% male and 50% female. In some embodiments, a majority of the adults of the aquatic insect that the apparatus yields are male. In some embodiments, at least about 10%, at least about 20%, at least about 30%, at least about 40%, at least about 50%, at least about 60%, at least about 70%, at least about 75%, at least about 80%, at least about 85%, at least about 90%, at least about 95%, at least about 99%, or 100% of the adults of the aquatic insect that the apparatus yields are male.

In some embodiments, the insect eggs disclosed herein contain an artificial Wolbachia infection, and the insect can be a Culicidae (mosquito) species, such as species within the subfamilies Culicinae and Anophelinae, e.g., Aedes albopictus, Aedes aegypti, and Aedes polynesiensis. Wolbachia are natural bacteria present in up to 60% of insect species, including some mosquitoes. However, Wolbachia is not usually found in the Aedes aegypti mosquito, the primary species responsible for transmitting human viruses such as Zika, dengue, chikungunya and yellow fever. As such, mosquitoes may be artificially infected by a strain of Wolbachia which does not normally or naturally infect the selected mosquito species. For instance, cytoplasm of infected mosquito embryo can be microinjected into target mosquito embryo to generate the artificial bacteria infected mosquito. When an infected male mosquito is crossed with an uninfected female mosquito or a female mosquito with a different infection type, a reduced number of offspring will be generated. As such, certain mosquito population control methods employ release of male mosquitoes infected with Wolbachia to mate with wild uninfected female mosquitoes to suppress a wild mosquito population.

When an infected female is crossed with an uninfected male or a male infected with a similar infection, infected offspring will be generated. As such, certain mosquito population control methods based on artificial Wolbachia infection allow both male and female adult mosquitoes to emerge, and the artificially infected males and females are introduced into a target mosquito population to control the reproduction capability of the population. Over time, the percentage of mosquitoes carrying Wolbachia grows until it remains high without the need for further releases. Mosquitoes with Wolbachia have a reduced ability to transmit viruses to people, including Zika, dengue, chikungunya and yellow fever. As explained above, such methods are efficient when both infected males and infected females are released, such that both males and females in the target population can mate with effected mosquito to propagate infected offspring, thereby limiting mosquito-borne pathogens to control or prevent mosquito-borne diseases such as dengue and Zika.

In some embodiments, the insect eggs disclosed herein do not contain an artificial Wolbachia infection.

In some embodiments, the genome of the eggs of the mosquito comprises a splice control sequence capable of directing sex-specific splicing of a polynucleotide encoding a functional gene product, optionally wherein the functional gene product is a tTAV or homolog thereof, a tTAV2 or homolog thereof, a tTAV3 or homolog thereof, or a tTAF or homolog thereof.

In some embodiments, the genome of the eggs of the mosquito further comprises a transcription control element that controls transcription by the presence or the absence of a chemical ligand, optionally wherein the transcription control element is a tetracycline-responsive element.

In some embodiments, sex-specific splicing results in female-specific lethality of the eggs or larvae, pupae, or adults therefrom.

In some embodiments, the aquatic insect is a sterilized insect. In some embodiments, the aquatic insect is an engineered insect. In some embodiments, the insect is engineered to contain a self-limiting gene. In some embodiments, the self-limiting gene has a lethal, deleterious, or sterilizing effect on the aquatic insect. In some embodiments, the self-limiting gene has a lethal, deleterious, or sterilizing effect on the offspring of the aquatic insect. In some embodiments, activation of the self-limiting gene is controlled by the presence or absence of a substance, such as tetracycline. In some embodiments, in the absence of the substance, the self-limiting gene is activated. In some embodiments, the insect can live and reproduce normally when fed a diet containing the substance, such as tetracycline, and can therefore be bred. In some embodiments, the self-limiting gene is expressed in a sex-specific manner, e.g., predominantly in females or in females only. In some embodiments, only male pupae eclose to adults, that is, only adult males emerge from their pupal cases. In some embodiments, the genetically-engineered males are released to mate with wild-type females. In some embodiments, mating of the engineered males to wild-type females results in progeny that inherit the self-limiting gene which does not permit them to mature to adult. In some embodiments, the engineered insect also contain a heritable, fluorescent marker to distinguish them from native pest insects and/or to help with the management of pest control programs.

In some embodiments, the aquatic insect is a pest. In some embodiments, the aquatic insect is a pest that spreads human disease. In some embodiments, the aquatic insect is a pest that damages crops. In some embodiments, the aquatic insect is a mosquito. In some embodiments, species of mosquitoes that may be released using the apparatus include, but are not limited to, mosquitoes of a genera selected from the group consisting of Anopheles sp., Culex sp., Aedes sp., and Toxorhynchites sp. In particular embodiments, the mosquitoes are selected from Anopheles fluviatilis, Culex quinquefasciatus, Anopheles strode, Anopheles pseudopuncti, Aedes aegypti, Anopheles shannoni, Anopheles apicimaculata, Aedes rubrithorax, Anopheles argyritarsis, Anopheles neomaculipal, Anopheles fluminensis, Aedes alboannulatus, Aedes albopictus, Anopheles punctimaculata, Anopheles anomolophyllus, Anopheles vestitipennis, Anopheles albimanus, Anopheles stephensi, Anopheles gambiae, Anopheles arabiensis, Anopheles funestus, Anopheles darlingii, Toxorhynchites brevipalpie, Toxorhynchites splendens, Toxorhynchites ambionensis, Toxorhynchites rutilus, and Toxorhynchites moctezumai. In specific embodiments, the mosquitoes are selected from the group consisting of Aedes aegypti, Aedes rubrithorax, Aedes albopictus, and Aedes alboannulatus.

In some embodiments, the insects disclosed herein can live and reproduce normally when they are fed a diet containing a chemical substance (e.g. tetracycline or an analog or derivative thereof, or a similar chemical compound) and can therefore be bred. However, in the absence of the chemical substance, a self-limiting gene is activated. In some embodiments, the insects disclosed herein are Aedes aegypti mosquito, and male pupae may be separated from female pupae, where only male pupae are allowed to eclose to adults. The genetically-engineered Aedes aegypti males can then be released to mate with wild-type female Aedes aegypti mosquitoes. The progeny inherit the self-limiting gene which does not permit them to mature to adult mosquitoes.

In some embodiments, the insect eggs disclosed herein comprise a gene expression system that imparts homozygous, sex-specific lethality in insects, such as female-specific lethality in mosquitoes. In some embodiments, in addition to female-specific lethality in the offspring, the insect strain can additionally be engineered such that the male offspring are sterile. The sterile males may be released to mate with wild female to suppress propagation of the insect population.

In some embodiments, the insects disclosed herein can also contain a heritable, fluorescent marker to distinguish them from native pest insects and to help scientists with the management of pest control programs.

The insect eggs can be generated using any suitable genetic engineering method, including but not limited to a method involving a transposable element (e.g., piggyBac or Sleeping Beauty), meganuclease, zinc finger nuclease (ZFN), site specific recombinase (e.g., a Cre-LoxP or Flp-FRT system), a transcription activator-like effector nuclease (TALEN), a CRISPR (e.g., a CRISPR/Cas system such as CRISPR/Cas9a/b/c or CRISPR/Cas12a/b or CRISPR/Cas13a/b/c/d), or any combination thereof (e.g., a transposon-associated CRISPR-Cas system such as CRISPR-transposons). The insect eggs can also be engineered for use in any suitable insect population suppression and/or modification approach, including but not limited to genetic-based approaches, such as various gene drive (GD)-based approaches and other approaches including pgSIT. RIDL, and fsRDIL. For instance, the eggs can carry gene drives that selfishly enable their spread without necessarily conferring a selective advantage to their carriers. Examples include homing-based gene drives (HGDs), sex-linked meiotic drives (e.g., sex-distorter gene drive programmed to home into dsx and express an endonuclease that shreds the X-chromosome), gene drives based on Medea, and various under dominance systems. For instance, in CRISPR HGDs, Cas9 can be guided by a programmable gRNA to generate a double stranded break (DSB) in a precise location, which is then repaired using the cell's homology-directed repair (HDR) machinery. When this process occurs in the germline of heterozygous individuals, wild-type alleles can be converted into drive alleles, thereby enabling super-Mendelian inheritance of the drive allele. In other embodiments, the insect eggs are for use in RIDL, a self-limiting approach, which involves a dominant lethal gene that utilizes modified components of the Tet-OFF operon system. In the absence of tetracycline, transactivator or an analog thereof binds to the operon sequence to induce toxic product expression in a tissue- and temporal-specific manner, where high concentrations of toxic products lead to lethality. In some embodiments, the insect eggs are for use in fsRIDL, a similar approach to RIDL, with added sex-specificity. A sex-specific intron ensures that transactivator or an analog thereof can express only in flight muscles of females to prevent them from flying. In some embodiments, the insect eggs produce transgenic mosquitoes carrying components encoding Cas9 and several gRNAs targeting sex-determination genes to enable the production of sterile male offspring. In some embodiments, the insect eggs are used for self-limiting split drive which involves separating both Cas9 and gRNA/GD element components to enable a safe, noninvasive, self-limiting system. Suitable genetic control technologies that can use the apparatus, device, kit, and system disclosed herein can include any described in Wang et al., Nature Communications 12: 4388 (2021), incorporated herein by reference in its entirety for all purposes.

### C. Diet Compositions

In some embodiments, provided herein are nutrient compositions (e.g., diet) formulated to support the yield of adult aquatic insects, such as mosquitoes. In some embodiments, provided herein are nutrient compositions to support the growth and development of mosquitoes from the egg to the larval stage, from the larval to the pupal stage, and/or from the pupal to the adult stage.

In some embodiments, the diet is formulated to support development of the aquatic insect from egg to larva, to pupa, and/or to adulthood. In some embodiments, the diet is formulated to minimize spoilage of the hydrating composition. In some embodiments, the diet is formulated to dissolve and/or disintegrate in a controlled manner when contacted with the hydrating composition.

In some embodiments, the diet is formulated as pellets. In some embodiments, the diet comprises a single pellet type or multiple pellet types. In some embodiments, the single pellet type comprises the same or different diet compositions. In some embodiments, the multiple pellet types comprise the same or different diet compositions. In some embodiments, the diet pellets comprise pellets designed for feeding to various animals, such as shrimp, guinea pig, or fish (e.g., carp and/or tilapia). In some embodiments, the diet pellets may comprise an alga such as spirulina, for instance, for larvae of insects such as mosquitoes of the genus Anopheles.

In some embodiments, the diet pellets are high in fat/oil content. In some embodiments, the diet pellets are low in fat/oil content. In some embodiments, the diet pellets are high in protein content. In some embodiments, the diet pellets are low in protein content. In some embodiments, the diet pellets comprise less than about 5% (w/w), less than about 10% (w/w), less than about 15% (w/w), or less than about 20% (w/w) fat/oil. In some embodiments, the diet pellets comprise about 15% (w/w), about 20% (w/w), about 25% (w/w), about 30% (w/w), about 35% (w/w), about 40% (w/w), about 45% (w/w), about 50% (w/w), about 55% (w/w), about 60% (w/w), about 65% (w/w), about 70% (w/w), or about 75% (w/w) protein. The protein can be from one or more microbial sources, one or more plant sources, and/or one or more animal sources. In some embodiments, the diet pellets comprise less than about 15% (w/w) fat/oil and between about 15% (w/w) and about 60% (w/w) protein. In some embodiments, the diet pellets comprise less than about 12% (w/w) fat/oil and between about 18% (w/w) and about 50% (w/w) protein. In some embodiments, the diet pellets comprise less than about 10% (w/w) fat/oil and between about 20% (w/w) and about 45% (w/w) protein. In some embodiments, the diet pellets comprise less than about 10% (w/w) fat/oil and between about 25% (w/w) and about 40% (w/w) protein. In some embodiments, the diet pellets comprise less than about 5% (w/w) fat/oil and between about 25% (w/w) and about 40% (w/w) protein.

In some embodiments, the diet may comprise about 1% (w/w), about 2% (w/w), about 5% (w/w), about 10% (w/w), about 15% (w/w), or higher water content, or a water content between any of the aforementioned values. In some embodiments, the diet is formulated and/or conditioned to provide moisture in the sealed receptacle. In some embodiments, the water content in the diet contributes to maintaining an internal relative humidity of the sealed receptacle that maintains viability of the eggs.

In some embodiments, the diet may comprise one or more components derived from a microorganism, one or more components derived from a plant, one or more components derived from an animal, or one or more inorganic components, or any combination thereof. In some embodiments, the diet pellets may comprise any one or more of dehulled soybean meal, ground corn, fish meal, fish oil, mineral oil, wheat middlings, wheat flour, corn gluten, rice hulls, calcium carbonate, yeast (e.g., brewers dried yeast), dehydrated alfalfa meal, sodium chloride, betaine (hydrochloride or anhydrous), a source of Vitamin A (e.g., Vitamin A acetate), a source of Vitamin B (e.g., a Vitamin B-12 supplement), a source of Vitamin C (e.g., L-ascorbyl-2-polyphosphate), a source of Vitamin E (e.g., DL-α tocopheryl acetate), a source of Vitamin K (e.g., menadione sodium bisulfite complex), a preservative (e.g., propionic acid and/or ethoxyquin), an alga such as spirulina, biotin, choline chloride, calcium pantothenate, zinc sulfate, zinc oxide, niacin, nicotinic acid, cholecalciferol, pyridoxine hydrochloride, folic acid, thiamine mononitrate, riboflavin, rosemary extract, citric acid, dicalcium phosphate, DL-methionine, manganous oxide, ferrous carbonate, ferrous sulfate, copper sulfate, calcium iodate, cobalt carbonate, sodium selenite, and a proteinate (e.g., zinc proteinate, copper proteinate, manganese proteinate, and/or iron proteinate). In some embodiments, any one or more of the aforementioned components (in any suitable combination), e.g., spirulina, may be provided separately from diet pellets comprising one or more other components. In some embodiments, the diet comprises TetraOBond™ Koi Vibrance. In some embodiments, the diet comprises Sera™ Koi Summer. The aforementioned components are non-limiting examples and may be used (e.g., included in the diet pellets) in any suitable combination, and may be used with other components not specifically mentioned herein.

In some embodiments, the diet pellets are formulated such that the pellet breakdown rate facilitates slow release of nutrients into the hydrating composition (e.g., water) over a period of time. In some embodiments, the diet is completely or partially coated with a coating, such as shellac. In some embodiments, the diet is completely or partially enclosed by a substance for slow feed release. In some embodiments, the diet can be in the form of a pallet inserted in a block of plaster of Paris configured to release the feed in the hydrating composition (e.g., water) over an extended period of time. In some embodiments, a diet pellet can be completely or partially coated, e.g., with a coating comprising shellac. In any of the embodiments herein, the diet can be completely or partially coated with one or more layers of coating, optionally wherein the coating comprises carnauba wax. In any of the embodiments herein, the diet can be completely or partially coated with one or more layers of coating, optionally wherein the coating comprises beeswax. In some embodiments, the coating is configured to control the rate at which the diet dissolves and/or disintegrates when contacted with the hydrating composition. In some embodiments, the thickness of the coating is a determinant of the rate at which the diet dissolves and/or disintegrates when contacted with the hydrating composition. In some embodiments, the material type, composition, shape, and/or configuration of the coating or enclosing material can be designed, selected, and/or adjusted to provide a rate at which the diet dissolves and/or disintegrates when contacted with the hydrating composition. In some embodiments, the rate at which the diet dissolves and/or disintegrates in the hydrating composition is slowed by the coating or enclosing material such that spoilage of the hydrating composition is reduced. In some embodiments, the coating or enclosing material comprises shellac. In any of the embodiments herein, the diet can be completely or partially coated with one or more layers of coating, optionally wherein the coating comprises carnauba wax. In any of the embodiments herein, the diet can be completely or partially coated with one or more layers of coating, optionally wherein the coating comprises beeswax. In some embodiments, the coating or enclosing material comprises plaster of Paris. In some embodiments, the coating comprises imperfections such as cellulose fibers. The diet may be coated with one or more different coating materials and/or one or more different coating layers. In some embodiments, the diet can be partially or completely coated, and the partial or complete coating can be configured to dissolve or otherwise disintegrate upon contacting water.

In some embodiments, the diet comprises a combination of diet pellets with different diet compositions, sizes, and/or coatings. In some embodiments, the combination of diet pellets are configured for different rates of break-down (e.g., the rate at which the diet dissolves and/or disintegrates), and/or for supporting larvae at different developmental stages. In some embodiments, the diet comprises a combination of a first pellet type and a second pellet type. In some embodiments, the first pellet type is configured for rapid break-down and supporting early larval development. In some embodiments, the second pellet type is configured for slow break-down and supporting late larval development. The first and second pellet type can be independently selected as needed.

In some embodiments, the diet comprises a plurality of pellets coated by the same coating or by different coatings. In some embodiments, the plurality of pellets comprise the same or different diet compositions.

In some embodiments, the diet is formulated and/or conditioned to provide moisture in the sealed receptacle. In some embodiments, the diet contributes to maintaining an internal relative humidity of the sealed receptacle that maintains viability of the eggs. In some embodiments, the diet contributes to maintaining an internal relative humidity of the sealed receptacle between about 55% and 90%. In some embodiments, the diet and/or the eggs are conditioned to maintain the internal relative humidity of the sealed receptacle between about 55% and about 60%, between about 60% and about 65%, between about 65% and about 70%, between about 70% and about 75%, between about 75% and about 80%, or between about 85% and about 90%. In some embodiments, a diet composition (e.g., diet pellets) can be conditioned in specific conditions, including relative humidity, temperature, and period (e.g., about 24 hours, 48 hours, 72 hours, or longer), based on the diet selected to ensure desired moisture level and the ability of the diet to maintain the internal relative humidity of a sealed receptacle containing aquatic insect eggs such as mosquito eggs.

While the diet compositions are described in connection with the receptacle, it should be appreciated that diet can be provided separately (e.g., outside the receptacle or in a receptacle that is separate from the receptacle comprising eggs). For instance, as an alternative or in addition to providing diet in the receptacle, diet can be added in the hydrating composition such as water near the receptacle by a user. In some embodiments, the diet and the eggs can be provided in different receptacles.

### III. COMPOSITIONS FOR EGG HATCHING

Once a receptacle comprising eggs is installed inside a housing/container and a hydrating composition is added to wet the eggs and at least a portion of the diet, the housing may be closed and eggs are allowed to hatch inside the closed housing.

FIG. 7 shows an exemplar apparatus 700 with the housing 720 in an open configuration, where the container 740 is placed in the housing. Both the receptacle 760 and water preservative capsule(s) 752 are submerged in water 770, below water fill line 772. In some aspects, water enters the receptacle and wets the eggs and at least a portion of the diet in the receptacle. In some aspects, water also wets a hatch stimulant which may be placed in the vicinity of the eggs in the receptacle. In some aspects, water that enters the receptacle contains a hatch stimulant already dissolved and/or dispersed therein such that hatching is triggered upon the eggs contacting the water, e.g., after the water is sufficient to cover the eggs. In some aspects, water that enters the receptacle can comprise one or more diet and/or nutrients for the insect, such as sugar that mimics nectar from flowers for male mosquitoes to feed on. Wetting of the diet (e.g., in the form of coated or uncoated pellets or other suitable form) also helps dissolving and/or dispersing the diet and nutrients in the water for larvae to feed on.

In a variety of ways described herein, hatching of the eggs in the receptacle can be synchronized, thereby synchronizing further development of larvae hatched from the eggs. In some embodiments, a hatch stimulant is provided to synchronize egg hatching inside the receptacle. In some embodiments, the hatch stimulant is provided in the egg section and can be mixed with or separated from the eggs in the egg section. In some embodiments, a hatch stimulant may be placed in the vicinity of the diet in the receptacle, for instance, the hatch stimulant can be mixed with or separated from the diet in the diet section. In some embodiments, a hatch stimulant may be placed in a section of a sealed receptacle other than the egg section and the diet section, and upon introduction of water into the receptacle, the hatch stimulant can be caused to contact the eggs and/or the diet, e.g., after the hatch stimulant dissolves in water. In some embodiments, a hatch stimulant may be placed in the vicinity of a receptacle but outside the receptacle. In some embodiments, the hatch stimulant can be dissolved in water to wet the eggs. In some embodiments, the hatch stimulant dissolved in water can diffuse to the eggs in the receptacle.

The hatch stimulant can be provided in any suitable form, including but not limited to a solid, liquid, semi-solid, gel, syrup, paste, pellet, powder, agglomerate, granule, compressed material, reconstitutable solid, suspension, lump, particulate, bead, emulsion, or wet lump, or any combination or mixture thereof, any one or more of which may be coated (with one or more different coating materials and/or one or more different coating layers) or uncoated. In cases where the hatch stimulant is coated, the hatch stimulant can be partially or completely coated, and the partial or complete coating can be configured to dissolve or otherwise disintegrate upon contacting water.

In some embodiments, the hatch stimulant can comprise cysteine, ascorbic acid, or a salt or solvate thereof, or any combination thereof. In some embodiments, the cysteine concentration in the hydrating composition is between about 10 mM and about 125 mM. In some embodiments, the cysteine concentration between about 15 mM and about 100 mM, such as about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, or about 95 mM. In some embodiments, the ascorbic acid concentration in the hydrating composition is between about 1.5 mM and about 40 mM. In some embodiments, the ascorbic acid concentration is between about 3 mM and about 30 mM, such as about 6 mM, about 9 mM, about 12 mM, about 15 mM, about 18 mM, about 21 mM, about 24 mM, or about 27 mM.

### IV. COMPOSITIONS FOR WATER PRESERVATION

In some embodiments, the hydrating composition is predominantly water. In some embodiments, the hydrating composition is a water-based solution. In some embodiments, the hydrating composition is a water-based suspension. The hydrating composition can be selected, for instance to mimic the natural habitat of the particular species of the aquatic insect. In some cases, mosquito may lay eggs in clean or muddy water that has been stagnant for days—while Aedes genus mosquitoes tend to lay eggs in clean water, Anopheles and Culex genus mosquitoes may lay eggs in muddy or dirty waters that are rich in organic food source.

In some embodiments, nutrient compositions such as diet can spoil or promote spoilage, such as the spoilage of water. In some aspects, provided herein are compositions that support a high yield of adult aquatic insects without promoting spoilage by providing metered release of nutrients into water.

In some embodiments, the apparatus comprises a water preservative. In some embodiments, the water preservative is configured to establish and/or maintain the quality of the hydrating composition to provide suitable conditions for rearing of aquatic insects. In some embodiments, the water preservative comprises one or more capsules coupled (e.g. attached, fastened, clipped, secured, held) to one or more of the adapter structures on the container bottom wall and/or side wall. In some embodiments, the water preservative comprises powder. In some embodiments, the water preservative is comprised in the container, or is added to the container. In some embodiments, the water preservative is comprised in or coupled to the receptacle. In some embodiments, the water preservative can be added as a powder, tablets or pellets pre-loaded (e.g., packed) into the container, and/or as a separate additive dissolved and/or dispersed into the hydrating composition by the user. In some embodiments, water preservative(s) can be stored in sachets and sprinkled into the container and/or the hydrating composition by a user.

In some embodiments, the water preservative comprises sorbic acid or a salt or solvate thereof, or a chlorinated hydroxytriazine or a salt or solvate thereof. In some embodiments, the water preservative comprises citric acid or a salt or solvate thereof. In some embodiments, the water preservative comprises potassium sorbate and/or sodium dichloroisocyanurate and/or citric acid. In some embodiments, the water preservative may comprise a sorbic acid or a salt, solvate, or derivative (e.g., an ester) thereof; a dichloroisocyanuric acid or a salt, solvate, or derivative (e.g., an ester) thereof; a metabisulphite or a salt, solvate, or derivative thereof; bronopol or a derivative thereof; a benzoic acid or a salt, solvate, or derivative (e.g., an ester) thereof; a formic acid or a salt, solvate, or derivative (e.g., an ester) thereof; a chloric acid or a salt, solvate, or derivative (e.g., an ester) thereof; a nitrous acid or a salt, solvate, or derivative (e.g., an ester) thereof; a propionic acid or a salt, solvate, or derivative (e.g., an ester) thereof; and/or a citric acid or a salt, solvate, or derivative (e.g., an ester) thereof. In some embodiments, the preservative comprises potassium sorbate, sodium dichloroisocyanurate, sodium metabisulphite, bronopol, sodium benzoate, sodium formate, sodium chloride, sodium nitrite, sodium propionate, and/or citric acid.

In some embodiments, the container is configured to hold a specified volume of the hydrating composition. In some embodiments, the specified volume is between about 200 mL and about 10,000 mL, or between about 500 mL and about 1500 mL. In some embodiments, the hydrating composition comprises or is water. In some embodiments, the container comprises a volume indicator which indicates the specified volume of the hydrating composition. In some embodiments, the volume indicator comprises a fill line on the side wall of the container. In some embodiments water or a hydrating composition is added to the chamber until the surface of the water or hydrating composition is approximately level with the volume indicator and/or fill line.

In some embodiments, the apparatus is configured to provide a head space between the hydrating composition and the housing in a closed configuration, when the container is placed inside the housing and the hydrating composition is in the chamber. In some embodiments, the head space provides sufficient space for an adult of the aquatic insect to survive prior to exiting the apparatus. In some embodiments, the head space can be adjusted by adjusting the configuration of the housing. In some embodiments, the head space can remain substantially the same whereas the size and/or the shape of the housing may vary.

The quality of available water can vary widely among geographic locales. In some examples, water quality refers to chemical and biological characteristics, such as pollution or contamination. Even when purified water is available, there exists a possibility of contamination from the surrounding ambient environment and/or components of the system, such as the nutritional compositions.

In some embodiments, provided herein are compositions that improve, such as purify, and/or maintain, such as preserve, water quality. In some aspects, provided herein are compositions that improve, such as purify, and maintain, such as preserve, water quality.

In some embodiments, the compositions for water purification and/or preservation are encapsulated within a capsule, such as a two piece system wherein an elongated cap closes or fits securely over the body of the second piece. The capsule encapsulating any of the provided water purification and/or preservation compositions can be any suitable material, including those disclosed herein. In some embodiments, the composition of the capsule itself can be selected or manipulated to provide a preferred effect, such as an optimal dissolution rate, and/or not being toxic to a selected aquatic insect species. In some embodiments, the capsule is a gelatin capsule, such as a fish gelatin capsule, a hypromellose capsule, a starch capsule, such as a potato starch capsule, a pullulan capsule, a polyvinyl acetate capsule. In some embodiments, the capsule is hard or soft, such as a soft or hard gelatin capsule. In some embodiments, the soft capsule is plasticized by glycerin, sorbitol, or a similar polyol. In some embodiments, the composition of the capsule itself comprises a preservative, such as a preservative to prevent the growth of microbiological contaminants.

In some embodiments, the capsule encapsulating any of the provided water purification and/or preservation compositions is clear or not colored. In some embodiments, the capsule encapsulating any of the provided water purification and/or preservation compositions is colored. In some embodiments, the size of the capsule encapsulating any of the provided water purification and/or preservation compositions is 000, 00, 0, 1, 2, 3, 4, or 5, with 000 having the largest size and 5 having the smallest size. Various capsule sizes and the weight or volume of the compositions they can encapsulate are known in the art. In one example, the length of a closed 00 sized capsule is about 0.917 inches (about 23.3 mm), which can encapsulate about 546 mg (of a powder with a density of 0.6 g/ml) or 0.91 ml of a provided composition. In some embodiments, the capsule encapsulating any of the provided water purification and/or preservation compositions comprises an inert filler substance. In some embodiments, the filler substance can comprise cellulose and/or calcium phosphate. In some embodiments, the filler substance is cellulose. In some embodiments, the filler substance is calcium phosphate.

In some embodiments, the compositions for water purification and/or preservation are formulated as a tablet or pellet, such as one-piece block wherein powders of the compositions are pressed into. The tablets or pellets containing any of the provided water purification and/or preservation compositions can be of any suitable material and manufactured by any suitable method. In some embodiments, the formulation of the tablets or pellets can comprise other compositions other than water preservative to provide a desirable effect, such as an optimal dispersion rate, and/or easier manufacturing procedures for the tablets or pellets. In some embodiments, the tablets or pellets containing any of the provided water purification and/or preservation compositions is clear or not colored. In some embodiments, the tablets or pellets containing any of the provided water purification and/or preservation compositions is colored. In some embodiments, the size of the tablets or pellets containing any of the provided water purification and/or preservation compositions is 000, 00, 0, 1, 2, 3, 4, or 5, with 000 having the largest size and 5 having the smallest size. Various tablet or pellet sizes and the weight or volume of the compositions therein can be used. In one example, the length of a closed 00 sized capsule is about 0.917 inches (about 23.3 mm), which can encapsulate about 546 mg (of a powder with a density of 0.6 g/ml) or 0.91 ml of a provided composition. In some embodiments, the tablets or pellets containing any of the provided water purification and/or preservation compositions comprises an inert filler substance. In some embodiments, the filler substance can comprise cellulose and/or calcium phosphate. In some embodiments, the filler substance is cellulose. In some embodiments, the filler substance is calcium phosphate. In some embodiments, the tablets or pellets containing any of the provided water purification and/or preservation compositions comprises a reagent to promote the distribution of any of the provided water purification and/or preservation compositions in the hydrating compositions. In some embodiments, the reagent is an effervescent reagent. In some embodiments, the effervescent reagent comprises bicarbonate or a salt or solvate thereof, such as sodium bicarbonate. In some embodiments, the effervescent reagent may comprise adipic acid or a salt or solvate thereof. In some embodiments, the effervescent reagent may comprise citric acid anhydrous or a salt or solvate thereof. In some embodiments, the tablets or pellets containing any of the provided water purification and/or preservation compositions comprises sodium dichloroisocyanurate, citric acid, sodium bicarbonate and cellulose. In some embodiments, the tablets or pellets containing any of the provided water purification and/or preservation compositions comprises potassium sorbate, citric acid, and sodium bicarbonate.

In some embodiments, the compositions for water purification and/or preservation comprises sorbic acid or a salt thereof. In some embodiments, the compositions for water purification and/or preservation comprises sorbic acid. In some embodiments, the compositions for water purification and/or preservation comprises potassium sorbate. In some embodiments, the compositions for water purification and/or preservation comprises calcium sorbate. In some embodiments, the compositions for water purification and/or preservation comprises sodium sorbate. In some embodiments, the composition comprising sorbic acid or a salt thereof is provided in one single unit, for example, in a single capsule. In some embodiments, the composition comprising sorbic acid or a salt thereof is provided in more than one unit, for example, in two, three, four, five, six, seven, eight, nine, 10, 20, 30, 40, 50, 60, 70, 80, 90, 100, or more than 100 capsules. In some embodiments, the composition comprises a total amount of sorbic acid or a salt thereof that is about 1 g-5 g, about 1 g-4 g, about 1 g-3 g, about 1 g-2 g, about 2 g-5 g, about 2 g-4 g, about 2 g-3 g, about 3 g-5 g, about 3 g-4 g, or about 4 g-5 g. about 1.5 g, about 1.6 g, about 1.7 g, about 1.8 g, about 1.9 g, about 2.0 g, about 2.1 g, about 2.2 g, about 2.3 g, about 2.4 g, about 2.5 g, about 2.6 g, about 2.7 g, about 2.8 g, about 2.9 g, about 3.0, about 3.1 g, about 3.2 g, about 3.3 g, about 3.4 g, or about 3.5 g. In some embodiments, the concentration of sorbic acid or a salt thereof in the hydrating composition (e.g., water) is about 0.2 g/L, about 0.4 g/L, about 0.6 g/L, about 0.8 g/L, about 1.0 g/L, about 1.2 g/L, about 1.4 g/L, about 1.6 g/L, about 1.8 g/L, about 2.0 g/L, about 2.2 g/L, about 2.4 g/L, about 2.6 g/L, about 2.8 g/L, about 3.0 g/L, or higher, or in a concentration between any of the aforementioned values, as long as the higher concentrations are not toxic to the selected aquatic insect species.

In some embodiments, the compositions for water purification and/or preservation comprise an organic acid, such as a carboxylic acid. In some embodiments, the compositions for water purification and/or preservation comprise a tricarboxylic acid. In some embodiments, the compositions for water purification and/or preservation comprise citric acid. In some embodiments, the composition comprising the organic acid, such as a carboxylic acid, is provided in one single unit, for example, in a single capsule. In some embodiments, the organic acid, such as a carboxylic acid, is provided in more than one unit, for example, in two, three, four, five, six, seven, eight, nine, or 10 capsules. In some embodiments, the composition comprises a total amount of organic acid, such as a carboxylic acid (e.g., citric acid), that is at least or about 10 mg, at least or about 20 mg, at least or about 30 mg, at least or about 40 mg, at least or about 50 mg, at least or about 60 mg, at least or about 70 mg, at least or about 80 mg, at least or about 90 mg, at least or about 100 mg, at least or about 200 mg, at least or about 300 mg, at least or about 400 mg, or at least or about 500 mg. In some embodiments, the composition comprises a total amount of organic acid, such as a carboxylic acid, that is about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, or about 70 mg. In some embodiments, the concentration of carboxylic acid (e.g., citric acid) in the hydrating composition (e.g., water) is about 1 mg/L, about 2 mg/L, about 5 mg/L, about 10 mg/L, about 20 mg/L, about 50 mg/L, about 100 mg/L, about 150 mg/L, about 200 mg/L, about 250 mg/L, about 300 mg/L, about 350 mg/L, about 400 mg/L, or higher, or in a concentration between any of the aforementioned values, as long as the higher concentrations are not toxic to the selected aquatic insect species.

In some embodiments, the compositions for water purification and/or preservation comprises a disinfectant. In some embodiments, the compositions for water purification and/or preservation comprises a chlorine-based disinfectant. In some embodiments, the compositions for water purification and/or preservation comprises sodium hypochlorite, calcium hypochlorite, or sodium dichloroisocyanurate. In some embodiments, the composition comprising the disinfectant is provided in one single unit, for example, in a single capsule. In some embodiments, the composition comprising the disinfectant is provided in more than one unit, for example, in two, three, four, five, six, seven, eight, nine, or 10 capsules. In some embodiments, the disinfectant is used at a concentration of about 5 mg/L, about 10 mg/L, about 15 mg/L, about 20 mg/L, about 25 mg/L, or about 30 mg/L, or any concentration between the aforementioned values, in the hydrating composition such as water.

In some embodiments, any combination of the described compositions can be used to provide water purification or preservation. In some embodiments, any of the provided compositions can be combined within the same unit, such as within the same capsule. In some embodiments, separate units comprising any of the provided compositions, such as single capsules, can be combined. In some embodiments, an organic acid, such as citric acid, and sorbic acid or a salt thereof, such as potassium sorbate, can be combined within the same unit. In some embodiments, separate units of an organic acid, such as citric acid, and sorbic acid or a salt thereof, such as potassium sorbate, can be combined.

In some embodiments, any combination of the described compositions can be used to provide water purification or preservation. In some embodiments, any of the provided compositions can be combined within the same unit, such as within the same tablet or pellet. In some embodiments, separate units comprising any of the provided compositions, such as single capsules, tablets, or pellets, can be combined. In some embodiments, an organic acid, such as citric acid, and sorbic acid or a salt thereof, such as potassium sorbate, can be combined within the same unit. In some embodiments, separate units of an organic acid, such as citric acid, and sorbic acid or a salt thereof, such as potassium sorbate, can be combined.

In some embodiments, one or more pH regulators can be provided in the hydrating composition such as water. In some embodiments, the water purification and/or preservation composition alters the pH of water. In some embodiments, the water purification and/or preservation composition comprises one or more pH regulators. In some embodiments, the water purification and/or preservation composition comprises pH regulators, wherein the pH regulator comprises a phosphate buffer system. In some embodiments, the pH regulator is based on a phosphate buffer system. In some embodiments, the water purification and/or preservation composition comprises one or more pH regulators, e.g., a Seachem™ pH regulator. In some embodiments, the one or more PH regulators comprise a Seachem™ neutral pH regulator. In some embodiments, the one or more pH regulators comprise a Seachem™ acid pH regulator. In some embodiments, the one or more pH regulators comprise a Seachem™ neutral pH regulator and a Seachem™ acid pH regulator. In some embodiments, the water purification and/or preservation composition comprises Seachem™ neutral pH regulator and Seachem™ acid pH regulator, wherein the mass ratio of the neutral pH regulator to the acid pH regulator is from 0.3 to 0.7, from 0.4 to 0.6, or about 0.5. In some embodiments, the water purification and/or preservation composition comprises Seachem™ neutral pH regulator that is about 1.5 g, about 1.6 g, about 1.7 g, about 1.8 g, about 1.9 g, about 2.0 g, about 2.1 g, about 2.2 g, about 2.3 g, about 2.4 g, or about 2.5 g per 570 mL of water. In some embodiments, the neutral pH regulator is at or about 0.0014 g/mL of water. In some embodiments, the water purification and/or preservation composition comprises Seachem™ acid pH regulator that is about 3.2 g, about 3.4 g, about 3.6 g, about 3.8 g, about 4.0 g, about 4.2 g, about 4.4 g, about 4.6 g, about 4.8 g, or about 5.0 g per 570 mL of water. In some embodiments, the acid pH regulator is at or about 0.0028 g/mL of water.

In some embodiments, the provided compositions alter the pH of the hydrating composition (e.g., water) to about 4.0-8.0, about 4.0-8.0, about 4.0-8.0, about 4.0-7.5, about 4.0-7.5, about 4.0-7.5, about 4.0-7.0, about 4.0-6.0, about 4.0-5.0, about 4.3-7.0, about 4.3-6.0, about 4.3-5.0, about 4.4-7.0, about 4.4-6.0, about 4.4-5.0, about 4.5-7.0, about 4.5-6.0, about 4.5-5.0, about 5.0-7.0, about 5.1-6.9, about 5.2-6.8, about 5.3-6.7, about 5.4-6.6, about 5.5-6.5, about 5.6-6.5, about 5.7-6.4, about 5.8-6.3, about 5.9-6.2, or about 6.0-6.5. In some embodiments, the provided compositions alter the pH of water to at least or about 4.0, at least or about 4.1, at least or about 4.2, at least or about 4.3, at least or about 4.4, at least or about 4.5, at least or about 4.6, at least or about 4.7, at least or about 4.8, at least or about 4.9, at least or about 5.0, at least or about 5.1, at least or about 5.2, at least or about 5.3, at least or about 5.4, at least or about 5.5, at least or about 5.6, at least or about 5.7, at least or about 5.8, at least or about 5.9, at least or about 6.0, at least or about 6.1, at least or about 6.2, at least or about 6.3, at least or about 6.4, at least or about 6.5, at least or about 6.6, at least or about 6.7, at least or about 6.8, at least or about 6.9, or at least or about 7.0.

In some embodiments, the hydrating composition (e.g., water) has an average pH value of about 4.0-8.0, about 4.0-8.0, about 4.0-8.0, about 4.0-7.5, about 4.0-7.5, about 4.0-7.5, about 4.0-7.0, about 4.0-6.0, about 4.0-5.0, about 4.3-7.0, about 4.3-6.0, about 4.3-5.0, about 4.4-7.0, about 4.4-6.0, about 4.4-5.0, about 4.5-7.0, about 4.5-6.0, about 4.5-5.0, about 5.0-7.0, about 5.1-6.9, about 5.2-6.8, about 5.3-6.7, about 5.4-6.6, about 5.5-6.5, about 5.6-6.5, about 5.7-6.4, about 5.8-6.3, about 5.9-6.2, or about 6.0-6.5, over a period of 2, 3, 4, 5, 6, 7, 8, 9, 10, 11, 12, 13, 14, 15, 16, 17, 18, 19, 20, 21, 22, 23, 24, 25, 26, 27, 28, 29, 30, or more days from hydration of the eggs in the apparatus. In some embodiments, the hydrating composition (e.g., water) has an average pH value of about 6.0-7.5 over the lifetime of the apparatus.

In some embodiments, the hydrating composition (e.g., water) comprises a pH regulator. In some embodiments, the pH regulator maintains an average pH value of the hydrating composition between about 5.8 and about 7.5. In some embodiments, the pH regulator maintains an average pH value of the hydrating composition between about 6.0 and about 7.0. In some embodiments, the pH regulator maintains an average pH value of the hydrating composition between about 6.3 and about 6.5. In some embodiments, the pH value of the hydrating composition (e.g., water) may change during the use of the apparatus, e.g., as the eggs hatch and mosquitoes develop and escape from the apparatus, the pH value of the hydrating composition can change over a 7-day period, a 14-day period, a 21-day period, or a 28-day period from hydration of the eggs in the apparatus. In some embodiments, the pH regulator maintains an average pH value of the hydrating composition over a 7-day period, a 14-day period, a 21-day period, or a 28-day period from hydration of the eggs in the apparatus, such that the pH value of the hydrating composition is maintained between about 5.8 and about 7.5, between about 6.0 and about 7.0, or between about 6.3 and about 6.5 in the various time periods.

In some embodiments, the hydrating composition such as water has a pH of about 6.4. In some embodiments, depending on the geographic locale (including the water pH at the geographic locale), one or more pH regulators can be used to regulate pH of the water in the container for mosquito development. In some embodiments, altering the pH to the described levels enhances the purifying and/or preserving activities of the provided compositions. In some embodiments, combining an organic acid, such as citric acid, and sorbic acid or a salt thereof, such as potassium sorbate, enhances the purifying and/or preserving activities of the provided compositions.

### V. COMPUTER SYSTEMS

The present disclosure provides computer systems that are programmed to implement and/or be used in connection with devices and methods of the disclosure. In some aspects, provided herein a computer system that is programmed or otherwise configured to implement methods of rearing and/or releasing an aquatic insect, for instance, by interfacing with a potential user or a user of the pest control apparatus, device, kit, or system disclosed herein. The computer system can regulate various aspects of the present disclosure, such as, for example, controlling the installation, operation, maintenance, and/or replacement of the pest control apparatus, device, kit, or system disclosed herein. The computer system can be an electronic device of a user or a computer system that is remotely located with respect to the electronic device. The electronic device can be a mobile electronic device.

In some embodiments, a computer system disclosed herein can comprise or be used with a non-transitory computer-readable medium comprising processor-executable instructions to cause a processor to: obtain information of a wild population of an arthropod in a geographic region, optionally wherein the processor-executable instructions cause the processor to forecast the information; provide instruction or recommendation to a user for placing an apparatus comprising eggs, pupae, larvae, and/or adults of bred arthropods at one or more locations in the geographic region based on the forecasted information, wherein adults of the bred arthropod emerge from the eggs, pupae, and/or larvae and/or exit the apparatus to mate with the wild population.

In some embodiments, a computer system disclosed herein can be used in one or more steps of a method comprising: forecasting information of a wild population of an arthropod in a geographic region; selecting a location in the geographic region, a date, and/or a time during the day based on the forecasted information; placing an apparatus comprising eggs of bred arthropods at the selected location, time during the day, and/or date; and allowing adults to emerge from the eggs in the apparatus, wherein the adults mate with the wild population. In some embodiments, the arthropod is a mosquito, and the forecasting comprises using: information regarding the arthropod; information regarding conditions in and/or outside the apparatus; information regarding weather and climate in the geographic region; terrain information in the geographic region; and/or information regarding human population and/or activity in the geographic region. Exemplary information that can be used for the forecasting includes but is not limited to: time, length of the larval stage, length of the pupal stage, time from hatching to adulthood, adult life expectancy, and/or flight range of the species of the arthropod; one or more diseases transmitted or borne by the species of the arthropod; crop or livestock damage caused by the species of the arthropod; the size of the wild population; the density of the wild population; the distribution of the wild population in the geographic region; the population size, density, and/or distribution of one or more predators of the arthropod in the geographic region; information regarding season, temperature, rainfall, humidity, and/or wind conditions in the geographic region; the amount and/or distribution of water bodies (e.g., stagnant water, rivers, lakes, ponds, tanks, reservoirs, and/or water treatment facilities) in the geographic region; and/or the size, density, distribution, demographic information, and/or personal information of the human population in the geographic region. In any of the embodiments herein, obtaining the information and/or the forecasting can be performed using one or more devices automatically, semi-automatically, or manually, including using a computer system or component(s) thereof disclosed herein. In any of the embodiments herein, obtaining the information and/or the forecasting can comprise using information obtained from one or more sources, such as one or more databases on the Internet and/or in the computer system or component(s) thereof.

In some embodiments, the computer system comprises a central processing unit (CPU), which can be a single core or multi core processor, or a plurality of processors for parallel processing. In some embodiments, the computer system comprises memory or memory location (e.g., random-access memory, read-only memory, flash memory), electronic storage unit (e.g., hard disk), communication interface for communicating with one or more other systems, and peripheral devices, such as cache, other memory, data storage and/or electronic display adapters. In some embodiments, the memory, storage unit, interface and peripheral devices are in communication with the CPU through a communication bus such as a motherboard. In some embodiments, the storage unit is a data storage unit or data repository for storing data. In some embodiments, the computer system is operatively coupled to a computer network with the aid of the communication interface. In some embodiments, the network is the Internet, an internet and/or extranet, or an intranet and/or extranet that is in communication with the Internet. In some embodiments, the network is a telecommunication and/or data network. In some embodiments, the network comprises one or more computer servers, which can enable distributed computing, such as cloud computing. The network, in some cases with the aid of the computer system, can implement a peer-to-peer network, which may enable devices coupled to the computer system to behave as a client or a server.

In some embodiments, the CPU of the computer system can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. The CPU can be part of a circuit, such as an integrated circuit. One or more other components of the system can be included in the circuit.

In some embodiments, the storage unit can store files, such as drivers, libraries, and saved programs. The storage unit can store user data, e.g., user preferences and user programs. The computer system in some cases can include one or more additional data storage units that are external to the computer system, such as located on a remote server that is in communication with the computer system through an intranet or the Internet.

In some embodiments, the computer system communicates with one or more remote computer systems through the network, including a local area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

In some embodiments, the computer system communicates with a remote computer system of a user (e.g., a cloud computing system, a server system). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system via the network.

In some embodiments, the computer system comprises a front end. In some embodiments, the front end is a native application (app) for a particular operating system or a web app. In some embodiments, the native app is a mobile app for e.g., Google, Android, or iOS. In some embodiments, the front end uses a JavaScript framework. In some embodiments, the front end platform links the app to a cloud service. In some embodiments, the front end is based on Angular. In some embodiments, the cloud service is based on Azure. In some embodiments, the system further comprises a back end. In some embodiments, the back end is a cloud service. In some embodiments, the cloud back end service comprises data storage, security and/or processing. In some embodiments, the app is a preconfigured app. In some embodiments, the preconfigured app comprises a user-friendly interface for a computer or mobile device, such as a companion web app.

Methods as described herein can be implemented by way of machine (e.g., computer processor) executable code stored on an electronic storage location of the computer system, such as, for example, on the memory or electronic storage unit. The machine executable or machine-readable code can be provided in the form of software. During use, the code can be executed by the processor. In some cases, the code can be retrieved from the storage unit and stored on the memory for ready access by the processor. In some situations, machine-executable instructions are stored on memory. The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Such processors may comprise, or may be in communication with, media, for example one or more non-transitory computer-readable media, that may store processor-executable instructions that, when executed by the processor, can cause the processor to perform methods according to this disclosure as carried out, or assisted, by a processor. Carrier-wave transmission media may take the form of electric or electromagnetic signals, or acoustic or light waves such as those generated during radio frequency (RF) and infrared (IR) data communications. Examples of non-transitory computer-readable medium may include, but are not limited to, an electronic, optical, magnetic, or other storage device capable of providing a processor, such as the processor in a web server, with processor-executable instructions. Other examples of non-transitory computer-readable media include, but are not limited to, a floppy disk, CD-ROM, magnetic disk, memory chip, ROM, RAM, ASIC, configured processor, all optical media, all magnetic tape or other magnetic media, or any other medium from which a computer processor can read. Many of these forms of computer readable media may be involved in carrying one or more sequences of one or more instructions to a processor for execution. The processor, and the processing, described may be in one or more structures, and may be dispersed through one or more structures. The processor may comprise code to carry out methods (or parts of methods) according to this disclosure.

Methods and systems of the present disclosure can be implemented by way of one or more algorithms. An algorithm can be implemented by way of software upon execution by the central processing unit. The algorithm can, for example, determine optimal conditions for implementing the methods and using the compositions of the present disclosure.

In some embodiments, the computer system can be or is in communication with an electronic display that comprises a user interface (UI) for providing, for example, an interface to start and/or monitor the progress of egg hatching, insect development, and/or insect deployment. Examples of UI's include, without limitation, a graphical user interface (GUI) and web-based user interface.

In some embodiments, the pest control apparatus, device, kit, or system (including the computer system) disclosed herein can be used in combination with a customer relationship management (CRM) software. In some embodiments, the pest control apparatus, device, kit, or system can be used in combination with an enterprise resource planning (ERP) software. In some embodiments, the pest control apparatus, device, kit, or system can be used in combination with a software comprising CRM and ERP. In some embodiments, the CRM and/or ERP software is used to manage business activities. Non-limiting examples of business activities include management of customers, orders (e.g., logistics operation and/or fulfilment), and contracts. In some embodiments, the pest control apparatus, device, kit, or system is used in connection with an ERP/CRM software, which may but does not need to be based on MS Dynamics.

In some embodiments, the app provides an interface facilitating the user to create an account or sign up. In some embodiments, the app provides an interface facilitating a returning user to sign in, such as by signing in with a user name/username or email address and a password. In some embodiments, the app provides an interface facilitating the user returning to recover a forgotten password.

In some embodiments, the app provides the user with one or more option(s) for a product and/or a service for in-app purchases, including for example, discounts on products and/or services as well as incentive to use and/or purchase the products and/or services. In some embodiments, the app displays the product and/or service for purchase, such as a description of the selected product and/or service, the price of the selected product and/or service, and the total cost associated with the same. In some embodiments, the app provides the user with options for payment. In some embodiments, the app provides the user with options for sponsoring a geographic region to be treated, e.g., using one or more apparatuses comprising eggs, pupae, larvae, and/or adults of bred arthropods (e.g., sterilized mosquitoes or genetically engineered self-limiting mosquitoes) disclosed herein. In some embodiments, the app provides the user with tracking data on the shipped product(s).

In some embodiments, the app provides an interface for the user to input data. In some embodiments, the app provides an interface for the user to input previous customer use data. In some embodiments, the app provides an interface for the user to input information about areas, for example, a local area's mosquito control activities. In some embodiments, the app provides an interface for the user to input data from historic records. Non-limiting examples of historic records include previous surveys, data from municipalities, and/or mosquito collections.

In some embodiments, the app provides an interface for the user to input disease data from health districts and/or hospitals. In some embodiments, the app provides an interface for the user to input demographic data on human populations. In some embodiments, the app provides an interface for the user to input apparatus and/or receptacle data. In some embodiments, the app provides an interface for the user to input release data.

In some embodiments, the app provides an interface for the user to input data on current and past trapping data. In some embodiments, trapping data comprises data from egg traps. In some embodiments, trapping data comprises data from adult traps. In some embodiments, the app provides an interface for the user to input locations that have previously been treated, are currently being treated, and/or where release is ongoing. In some embodiments, the app provides an interface for the user to input satellite imagery, area locations, population/housing density, and/or polygons. In some embodiments, the app provides an interface for the user to input information about mosquito production and/or quality control, for example, batch release data. In some embodiments, the app provides an interface for the user to input information on weather, for example, wind, rainfall, and temperature.

In some embodiments, the app comprises a mapping unit. In some embodiments, the mapping unit comprises a system to process data. In some embodiments, the mapping unit comprises ArcGIS. In some embodiments, the mapping unit comprises a non-ArcGIS system. In some embodiments, the mapping unit comprises one or more Microsoft power apps. In some embodiments, the mapping unit comprises an analytical model such as a predictive model. In some embodiments, the mapping unit provides spatial analysis of historical data in addition to prediction. In some embodiments, the predictive model is based one or more factor(s). In some embodiments, the one or more factor(s) comprises weather and/or climate data. In some embodiments, the one or more factor(s) comprises ongoing release data from the targeted area(s).

In some embodiments, the mapping unit comprises modelling of distribution for areas without mosquito traps. In some embodiments, the mapping unit comprises modelling of distribution based on trap and weather data from similar areas. In some embodiments, the mapping unit ensures resolution of spatial granularity of data to create adjacent or overlapping polygons, enabling creation of real-time distribution mapping.

In some embodiments, the mapping unit displays a graphical image of apparatus and/or receptacle location(s) and apparatus and/or receptacle status within an area selected by the user. In some embodiments, the apparatus and/or receptacle status comprises an indicator, such as a color and/or an icon, of whether the apparatus and/or receptacle has been deployed, when the cartridge of the apparatus and/or receptacle should be changed, and/or when whether the apparatus and/or receptacle should be disposed of. In some embodiments, the mapping unit displays a graphical image of apparatus and/or receptacle locations and/or free spots within an area selected by the user. In some embodiments, more than one area can be selected using the mapping unit. In some embodiments, the mapping unit provides the user with recommendations. In some embodiments, the mapping unit provides the user with recommendations on dosage for one or more area(s) selected by the user. In some embodiments, the mapping unit provides the user with recommendations on placement of apparatus and/or receptacles for one or more area(s) selected by the user.

In some embodiments, the app provides the user with multilingual outputs. In some embodiments, the app provides the user with outputs in Spanish, Tagalog, Portuguese, French, English, and/or other languages. In some embodiments, the app provides a graphic user interface (GUI) for the user to monitor purchased products, apparatus and/or receptacle location, pending orders, and to use a QR code scan function. In some embodiments, scanning the QR code provides the user with product- or purchase-related information, such as shipping status, location tracking, and/or product status and expiration.

In some embodiments, the app provides the user with instructions for use of the apparatus and/or receptacle. In some embodiments, the app provides step-by-step instructions or tutorials. In some embodiments, the app provides tutorials for installing and/or recharging the apparatus and/or receptacle. In some embodiments, the app provides tutorials for installing the apparatus and/or receptacle as a standalone unit or attached to a surface. In some embodiments, the tutorials comprise information on installing mounting brackets, attaching the apparatus and/or receptacle, securing the apparatus and/or receptacle, closing the lid of the apparatus and/or receptacle, and opening the lid of the apparatus and/or receptacle. In some embodiments, the app provides instructions for insect management, such as insect management after installing the apparatus and/or receptacle. In some embodiments, the app provides the user with user tutorials or answers to common questions, such as a help section. In some embodiments, the tutorial(s) is/are optional.

In some embodiments, the app provides the user with information on the location of one or more apparatus and/or receptacle(s) in an area. In some embodiments, the app provides the user with information on the performance of the apparatus and/or receptacle(s) in the area. In some embodiments, the app provides the user with information on the location of one or more apparatus and/or receptacle(s) and the performance of the apparatus and/or receptacle(s) in the area, for example, the effect on the native mosquito population. In some embodiments, the app may comprise a web app that provides a mechanism to select service companies for deployment and/or a dashboard to allocate tasks and track product deployment, and/or a mobile app for individual deployers to view tasks allocated and instructions for deployment.

In some embodiments, the app provides the user with information about local area's mosquito control activities. In some embodiments, the app provides the user with treatment rates and/or treatment failures in a particular area or location. In some embodiments, the app provides a value, such as a percentage, for the number of apparatus and/or receptacle(s) deployed and/or a value describing the area covered, such as a protected area, by the apparatus and/or receptacle(s).

In some embodiments, the app provides the user with one or more alert(s) or notification(s), such as an update or a prompt. In some embodiments, the app provides the user one or more alert(s) or notification(s), such as an actionable notification. In some embodiments, the one or more alert(s) or notification(s) is based on the user's activity, for example, purchasing activity.

In some embodiments, the one or more alert(s) or notification(s) inform the user on events based on aspects of arthropod (e.g., insect, such as mosquito) life cycle. Non-limiting examples of such events include egg expiration dates, time from apparatus and/or receptacle activation to mosquito maturity and release.

In some embodiments, the one or more alert(s) or notification(s) inform the user of time-sensitive actions. Non-limiting examples of time-sensitive actions include installation tasks, cartridge expiry date and/or when to change the internal cartridge, timing on replacement orders, such as when to re-order or re-stock consumables.

In some embodiments, the app provides the user with an interface to manage actionable notifications, for example, a task list and one or more options for assigning a task. In some embodiments, the app provides the user with one or more alert(s) or notification(s) related to a task, such as when a task should be performed and to whom a task is assigned. In some embodiments, the app provides the user with one or more recommendation(s). In some embodiments, the one or more recommendation(s) relates to dosage, such as a dosage recommendation based on an area selected by the user. In some embodiments, the app provides visualizations and/or dashboards to report on progress and/or trends pertaining to the insect control program and/or the device itself. In some embodiments, the app provides flexible branding and/or dynamic content, such as branding options for white labelling purposes as well as functionality to upload help, tutorial and marketing content.

While some examples of methods and systems herein are described in terms of software executing on various machines, the methods and systems may also be implemented as specifically-configured hardware, such as field-programmable gate array (FPGA) specifically to execute the various methods according to this disclosure. For example, examples can be implemented in digital electronic circuitry, or in computer hardware, firmware, software, or in a combination thereof. In one example, a device may include a processor or processors. The processor comprises a computer-readable medium, such as a random access memory (RAM) coupled to the processor. The processor executes computer-executable program instructions stored in memory, such as executing one or more computer programs. Such processors may comprise a microprocessor, a digital signal processor (DSP), an application-specific integrated circuit (ASIC), field programmable gate arrays (FPGAs), and state machines. Such processors may further comprise programmable electronic devices such as PLCs, programmable interrupt controllers (PICs), programmable logic devices (PLDs), programmable read-only memories (PROMs), electronically programmable read-only memories (EPROMs or EEPROMs), or other similar devices.

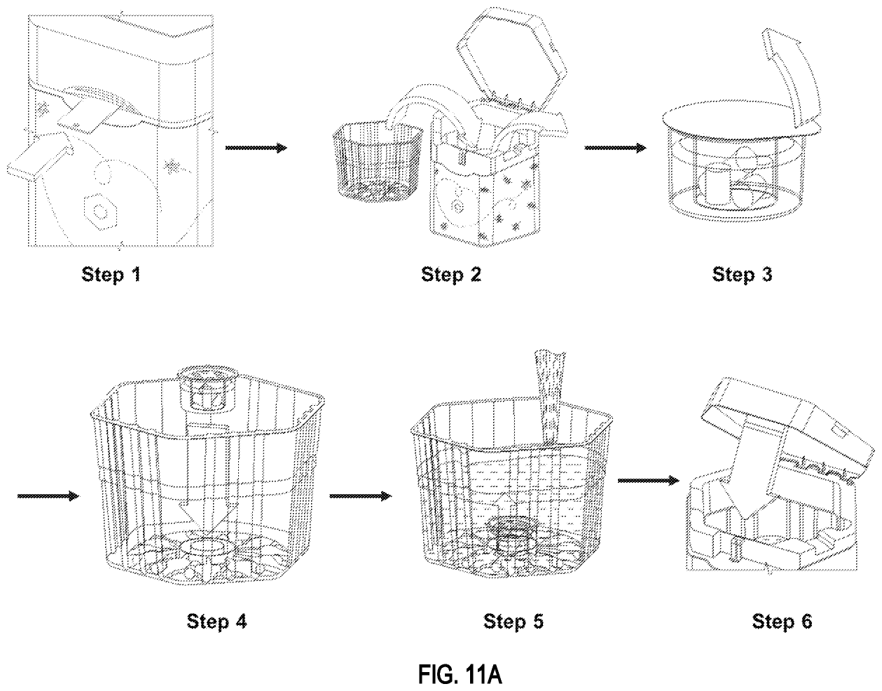

Referring to FIG. 10, device 1000 can be a host computer connected to a network. Device 1000 can be a client computer or a server. The device can be any suitable type of microprocessor-based device, such as a dedicated computing device, a personal computer, work station, server, handheld computing device (portable electronic device) such as a phone or tablet, an edge-AI device, or a neural network device. The device can include, for example, one or more of processors 1002, input device 1006, output device 1008, storage 1010, and communication device 1004. Input device 1006 and output device 1008 can generally correspond to those described above and can either be connectable or integrated with the computer. Input device 1006 can be any suitable device that provides input, such as a camera sensor, touchscreen, keyboard or keypad, mouse, or voice-recognition device. Output device 1008 can be any suitable device that provides output, such as an illuminator, a touchscreen, haptics device, or speaker. Storage 1010 can be any suitable device that provides storage, such as an electrical, magnetic, or optical memory including a RAM, cache, hard drive, or removable storage disk. Communication device 1004 can include any suitable device capable of transmitting and receiving signals over a network, such as a network interface chip or device. The components of the computer can be connected in any suitable manner, such as via a physical bus, or wirelessly.

Software 1012, which can be stored in storage 1010 and executed by processor 1002, can include, for example, the programming that embodies the functionality of the present disclosure (e.g., as embodied in the devices described above). Software 1012 can also be stored and/or transported within any non-transitory, computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1010, that can contain or store programming for use by or in connection with an instruction-execution system, apparatus, or device. Software 1012 can also be propagated within any transport medium for use by or in connection with an instruction-execution system, apparatus, or device, such as those described above, that can fetch instructions associated with the software from the instruction-execution system, apparatus, or device and execute the instructions. In the context of this disclosure, a transport medium can be any medium that can communicate, propagate, or transport programming for use by or in connection with an instruction-execution system, apparatus, or device. The transport readable medium can include, but is not limited to, an electronic, magnetic, optical, electromagnetic, or infrared wired or wireless propagation medium.

Device 1000 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines. Device 1000 can implement any operating system suitable for operating on the network. Software 1012 can be written in any suitable programming language. In various embodiments, application software embodying the functionality of the present disclosure can be deployed in different configurations, such as in a client/server arrangement or through a Web browser as a Web-based application or Web service, for example.

In some embodiments, provided herein is a method, comprising: obtaining information of a wild population of an arthropod (e.g., an insect, an arachnid such as ticks, etc.) in a geographic region; selecting a location in the geographic region, a date, and/or a time during the day based on the obtained information; placing an apparatus comprising eggs, pupae, larvae, and/or adults of bred arthropods at the selected location, time during the day, and date; and allowing adults to emerge from the eggs and/or exit the apparatus, wherein the adults mate with the wild population. In some embodiments, the arthropod is an insect, for instance, a mosquito.

In some embodiments, the method comprises obtaining information of the wild population of the arthropod by an automated method and/or a manual method, such as using a smart counting device and/or one or more traps for the arthropod. In some embodiments, the method comprises obtaining information of the wild population of the arthropod using forecasting. In some embodiments, the forecasting can comprise overall data analysis, such as regression and/or classification of historical data and/or data obtained at the time of the forecasting.

In some embodiments, the method comprises selecting a location in the geographic region based on the obtained information in order to place the apparatus at the location. In some embodiments, the method comprises selecting a date/time based on the obtained information in order to place the apparatus on the date/time. In some embodiments, the method comprises selecting a location in the geographic region and a time and date based on the obtained information in order to place the apparatus at the location and on the date and time. In some embodiments, the method uses weather/climate data to forecast and select when and where to place one or more of the apparatuses in the geographic region.

In some embodiments, the method comprises using information regarding the arthropod; information regarding weather and climate in the geographic region; terrain information in the geographic region; and/or information regarding human population and/or activity in the geographic region, in order to obtain information and make forecast to help select when and where to place one or more of the apparatuses in the geographic region for the purpose of controlling wild populations of an arthropod species in the geographic region.

In some embodiments, the method comprises using information regarding in-apparatus conditions, such as light, humidity, water, and airspace temperature, as well as information regarding the outside of the apparatus, such as installation photos, drone monitoring footage, etc. Such information can be used for selecting the apparatus or component(s) thereof (e.g., receptacles containing various numbers of eggs), monitoring the use of the apparatus, and/or selecting when and where to place one or more additional apparatuses in the geographic region.

In some embodiments, the method comprises using information regarding hatching time, length of the larval stage, length of the pupal stage, time from hatching to adulthood of the species of the arthropod, adult life expectancy, and/or flight range of the species of the arthropod, one or more diseases transmitted or borne by the species of the arthropod, and/or crop or livestock damage caused by the species of the arthropod. In some embodiments, the method comprises using information regarding the duration of adulthood of an insect, and in the case of flying insects such as mosquitoes, the flight range of adult insects, in order to inform placement distribution of the apparatus (e.g., one or more devices comprising eggs, pupae, larvae, and/or adults of genetically engineered, radiation-sterilized, and/or Wolbachia-infected mosquitoes). In some embodiments, the information is a reference or an estimate. In some embodiments, the information can be used to forecast and help select when and where to place one or more of the apparatuses in the geographic region for the purpose of controlling wild populations of an arthropod species in the geographic region.

In some embodiments, the method comprises using information regarding the size of the wild population, the density of the wild population, and/or the distribution of the wild population in the geographic region, and/or the population size, density, and/or distribution of one or more predators of the arthropod in the geographic region. In some embodiments, the method comprises using information regarding the one or more predators to estimate predation impact on the eggs, pupae, larvae, and/or adults of the bred arthropod. In some embodiments, the information comprises historical information and/or information obtained at the time of the forecasting. In some embodiments, the information can be used to forecast and help select when and where to place one or more of the apparatuses in the geographic region for the purpose of controlling wild populations of an arthropod species in the geographic region.

In some embodiments, the method comprises using information regarding season, temperature, rainfall, humidity, and/or wind conditions in the geographic region. In some embodiments, the information comprises historical information, a reference or an estimate, and/or information obtained at the time of the forecasting. In some embodiments, the information comprises annual or monthly mean temperatures and precipitations in the geographic region. In some embodiments, the information can be used to forecast and help select when and where to place one or more of the apparatuses in the geographic region for the purpose of controlling wild populations of an arthropod species in the geographic region.

In some embodiments, the method comprises using information regarding the amount and/or distribution of water bodies in the geographic region. In some embodiments, the water bodies comprise stagnant water, rivers, lakes, ponds, tanks, reservoirs, and/or water treatment facilities in the geographic region. In some embodiments, the terrain information is a reference or an estimate. In some embodiments, the information can be used to forecast and help select when and where to place one or more of the apparatuses in the geographic region for the purpose of controlling wild populations of an arthropod species in the geographic region.

In some embodiments, the method comprises using information regarding human population and/or activity is information regarding the size, density, distribution, demographic information, and/or personal information of the human population in the geographic region. In some embodiments, the information comprises historical information, a reference or an estimate, and/or information obtained at the time of the forecasting. In some embodiments, the information comprises data (e.g., demographic information and/or personal information) relating to age, gender, household income, and/or other potential factors influencing level of exposure of the human population to arthropods in the geographic region. In some embodiments, the information comprises data, e.g., physical and/or email addresses, of the hosts of the apparatus in the geographic region or hosts in other comparable geographic region(s). In some embodiments, the information comprises municipal information used to identify hotspots where the apparatus can be placed. In some embodiments, the information comprises information regarding historical spraying, disease hotspots, locations of rubbish dumps/cisterns/citations for fly tipping, etc. In some embodiments, the information can be from one or more datasets from governments, such as a municipal government. In some embodiments, the information can be used to forecast and help select when and where to place one or more of the apparatuses in the geographic region for the purpose of controlling wild populations of an arthropod species in the geographic region.

In some embodiments, the method comprises forecasting performed using one or more devices. In some embodiments, obtaining the information and/or the forecasting is performed automatically, semi-automatically, or manually, for instance, using one or more apps disclosed herein. In some embodiments, the forecasting comprises using information obtained from one or more sources. In some embodiments, the one or more sources comprise a local source in one or more devices configured to perform the forecasting and/or one or more internet servers, such as internet servers hosting government datasets regarding historical spraying, disease hotspots, and/or locations of rubbish dumps/cisterns/citations for fly tipping. In some embodiments, the forecasting is for about a week, about two weeks, about three weeks, about four weeks, or longer from the time of forecasting. In some embodiments, the forecasting can be for 2, 3, 4, 5, 6, 7, 8, 9, 10, 11, 12, 13, 14, 15, 16, 17, 18, 19, 20, 21, 22, 23, 24, 25, 26, 27, 28, 29, 30, or more days.

In some embodiments, the geographic region is selected using one or more devices, and the geographic region can be regular or irregular in shape. In some embodiments, using an app disclosed herein, a user can select and/or define the geographic region. In some embodiments, the geographic region is selected using the same device(s) for performing the forecast. In some embodiments, the geographic region is selected using device(s) different from device(s) for performing the forecast. In some embodiments, the selected geographic region is provided to device(s) for performing the forecast based on the selected geographic region. In some embodiments, the process or device for selecting the geographic region can exchange information with the process or device for performing the forecast, such that the forecast can be adjusted based on adjustments in the selection of geographic region, and/or the selection of geographic region can be adjusted based on adjustments in the forecast.

In some embodiments, the location in the geographic region is selected using one or more devices. In some embodiments, the location is selected using the same device(s) for performing the forecast and/or the same device(s) for selecting the geographic region. In some embodiments, the location is selected using different from device(s) for performing the forecast and/or device(s) for selecting the geographic region. In some embodiments, the forecasted information is provided to device(s) for selecting the location in the geographic region. In some embodiments, the process or device for selecting the geographic region, the process or device for performing the forecast, and/or the process or device for selecting the location in the geographic region can exchange information with one another, such that the forecast, the selection of geographic region, and/or the selection of the location with a geographic region can be adjusted based on adjustments in one another.

In some embodiments, a plurality of locations are selected in the geographic region. In some embodiments, the plurality of locations form a grid in which each location is a node. In some embodiments, the grid is a regular grid or irregular grid. In some embodiments, one or more of the apparatus is placed at each of the plurality of locations.

In some embodiments, the method comprises using ground-based data gathering to obtain population density data of the arthropod population in and/or around the geographical region. In some embodiments, the method comprises using the obtained population density data for the forecast.

In some embodiments, the method comprises delivering the apparatus comprising eggs, pupae, larvae, and/or adults of bred arthropods at or near the location. In some embodiments, the apparatus is delivered at or near the location using a ground distribution vehicle or an aerial craft. In some embodiments, the aerial craft is a piloted aircraft or a pilotless drone.

In some embodiments, the method comprises providing information of the apparatus to a user to place the apparatus. In some embodiments, the information of the apparatus comprises tracking formation. In some embodiments, the tracking formation is provided on a map on a device of the user. In some embodiments, the information of the apparatus comprises expiry information. In some embodiments, the method comprises providing information of one or more apparatuses that have been placed in the geographic region.

In some aspects, provided herein is a non-transitory computer-readable medium comprising processor-executable instructions to cause a processor to: obtain information of a wild population of an arthropod in a geographic region, optionally wherein the processor-executable instructions cause the processor to forecast the information; provide instruction or recommendation to a user for placing an apparatus comprising eggs, pupae, larvae, and/or adults of bred arthropods at one or more locations in the geographic region at one or more times during the day and/or one or more dates based on the forecasted information, wherein adults of the bred arthropod emerge from the eggs and/or exit the apparatus to mate with the wild population.

In some embodiments, the method and/or the non-transitory computer-readable medium disclosed herein uses an app disclosed herein to inform a user when and where to place one or more of the apparatus disclosed herein. In some embodiments, the method and/or the non-transitory computer-readable medium disclosed herein uses an app disclosed herein for commercial sales and/or restocking of the apparatus disclosed herein.

In some embodiments, the method and/or the non-transitory computer-readable medium disclosed herein is for use in controlling the wild population of the arthropod in the geographic region. In some embodiments, the adults that exit the apparatus are males. In some embodiments, the adults that exit the apparatus are genetically engineered male insects that mate with wild female insects of the same species in the geographic region. In some embodiments, the arthropod is a mosquito of the genus Aedes, Anopheles, or Culex.

The computer systems including apps disclosed herein can be used to perform one or more steps of the methods disclosed herein. For instance, any one or more of the steps of: obtaining information of a wild population of an arthropod in a geographic region; forecasting information of the wild population; selecting the geographic region; selecting one or more locations in the geographic region; selecting one or more times during the day; selecting one or more days; and selecting one or more devices (e.g., receptacles comprising various numbers of eggs) to be placed at the one or more locations, times during the day, and/or dates can be performed using a computer device or system disclosed therein. For example, any one or more or all of the aforementioned exemplary steps can be performed using an app disclosed herein.

### VI. KITS, DEVICES, AND METHODS OF USE

In some embodiments, provided herein are compositions and kits, for example, comprising one or more components of the apparatus or device described herein, including consumables, such as containers, receptacles, insect eggs, water preservatives, etc. The various components of the kit may be present in separate containers or packages, and certain compatible components may be precombined into a single container, structure, or package. In some embodiments, the kits can contain components including consumables required for performing one or more steps of the provided methods. In some embodiments, the kits further contain instructions for using the components including consumables of the kit to practice the provided methods.

In some embodiments, provided herein are methods of using the apparatuses, devices, kits, and systems, including one or more components thereof (including consumables), for rearing an aquatic insect from eggs through adulthood and for efficient, high density deployment of the adult insects. Disclosed herein is a method for rearing an aquatic insect using any apparatus or components thereof described above. In some embodiments, the method comprises: (a) inserting the receptacle into the container, thereby causing the receptacle to be coupled to the bottom wall of the container; (b) adding water to the chamber, whereby water enters the receptacle and contacts the eggs and the diet, thereby allowing the eggs to hatch in the receptacle.

In particular embodiments, the method comprises: (a) inserting a receptacle into a container, wherein: the container comprises a bottom wall and a side wall extending from the bottom wall, and the bottom wall and the side wall form a chamber for holding water, the receptacle comprises an egg section holding eggs of the aquatic insect and a diet section holding a diet composition for the aquatic insect, and the receptacle is affixed to the bottom wall of the container; (b) adding water to the chamber, whereby water enters the receptacle and contacts the eggs and the diet, thereby allowing the eggs to hatch in the receptacle.

In some embodiments, provided herein is a method for rearing an aquatic insects that comprises: (a) inserting a receptacle into a container, wherein: the container comprises a bottom wall and a side wall extending from the bottom wall, and the bottom wall and the side wall form a chamber for holding water; the receptacle comprises: a receptacle base comprising: (i) a receptacle bottom wall, (ii) a receptacle side wall extending from the receptacle bottom wall, and (iii) an enclosure, optionally extending from the receptacle bottom wall, wherein the receptacle side wall and/or the enclosure forms an egg section and a diet section within the receptacle base, and wherein the egg section and the diet section are connected, a receptacle lid comprising a plurality of perforations and engaging the receptacle base, and a receptacle seal forming an airtight seal with the receptacle base and/or the receptacle lid, wherein the receptacle seal is removed prior to or after inserting the receptacle into the container; and the receptacle is affixed to the bottom wall of the container; (b) adding water to the chamber, whereby the receptacle is submerged in water which enters the receptacle via the plurality of perforations and contacts the eggs and the diet, thereby allowing the eggs to hatch in the receptacle, allowing larvae to exit the receptacle via the plurality of perforations into the chamber, and/or allowing adults of the aquatic insect to emerge in the chamber.

In some embodiments, provided herein is a method for rearing an aquatic insects that comprises: (a) inserting a receptacle into a container, wherein: the container comprises a bottom wall and a side wall extending from the bottom wall, and the bottom wall and the side wall form a chamber for holding water; the receptacle comprises: a receptacle base comprising: (i) a receptacle bottom wall, and (ii) a receptacle side wall extending from the receptacle bottom wall, an enclosure comprising a tubular body and a flange disposed around the tubular body between a bottom end and an upper end of the tubular body, wherein the tubular body comprises a bottom opening, wherein the flange comprises a plurality of perforations, and wherein the flange engages the receptacle side wall, the receptacle side wall and/or the tubular body forms an egg section and a diet section within the receptacle base, the egg section and the diet section are connected, and the egg section is connected to a space above the flange via the plurality of perforations on the flange, and a receptacle seal configured to form an airtight seal with the receptacle base, wherein the receptacle seal is removed prior to or after inserting the receptacle into the container; and the receptacle is affixed to the bottom wall of the container; (b) adding water to the chamber, whereby the receptacle is submerged in water which enters the receptacle via the plurality of perforations on the flange, contacts the eggs, and, through the bottom opening of the tubular body, contacts the diet, thereby allowing the eggs to hatch in the receptacle, allowing larvae to exit the receptacle via the plurality of perforations on the flange into the chamber, and/or allowing adults of the aquatic insect to emerge in the chamber.

In some embodiments, the receptacle is inserted into the container, followed by adding water to the chamber to a predetermined level. In some embodiments, water is added to the chamber to a predetermined level, followed by inserting the receptacle into the container. In some embodiments, the egg section further comprises a hatch stimulant. In some embodiments, the receptacle is fully submerged in water. In some embodiments, the water enters the egg section and the diet section. In some embodiments, the water enters the egg section and the diet section at the same time. In some embodiments, the water enters the egg section prior to entering the diet section. In some embodiments, the water enters the diet section prior to entering the egg section. In some embodiments, the water enters the egg section via the diet section.

In some embodiments, the diet in the diet section is wetted prior to hatching of the eggs in the egg section. In some embodiments at least a portion of the diet in the diet section is kept dry until after hatching of the eggs in the egg section, until after larvae exit the receptacle, and/or until after adults of the aquatic insect emerge.

In some embodiments, the housing in the closed configuration maintains an average temperature of between about 10° C. and about 40° C. in the chamber and/or water therein. In some embodiments, the average temperature is between about 15° C. and about 32° C., or between about 20° C. and about 28° C. In some embodiments, the housing in the closed configuration is placed in shaded locations to maintain an average temperature of between about 20° C. and about 28° C. in the chamber and/or water therein. In some embodiments, the diet dissolves and/or disintegrates in a controlled manner, e.g., at a controlled rate in water. In some embodiments, at least about 50%, at least about 60%, at least about 70%, at least about 80%, at least about 90%, or at least about 95% of the diet is not dissolved and/or disintegrated within the first two, three, four, five, or six days of contacting the water. In some embodiments, the controlled release of nutrients in water provides a diet concentration in water that supports development of the aquatic insect and/or reduces spoilage of water compared to a higher diet concentration in water, for instance, between about 10° C. and about 40° C., such as between about 15° C. and about 32° C., in particularly between about 20° C. and about 28° C.

In some embodiments, the sealed receptacle contains between about 0.01 g and about 1 g, between about 0.01 and about 0.6 g, or between about 0.02 and 0.3 g eggs prior to water contacting the eggs. In some embodiments hatching of the eggs is synchronous and the container in the closed housing contains between about 500 and about 50,000, between about 1,000 and 25,000, or between about 1,000 and 15,000 larvae after the eggs hatch. In the case of mosquitoes, generally after molting three times, a larva becomes a pupa which will develop until the body of the newly formed adult flying mosquito emerges from the pupal skin and leaves the water. After adult mosquitoes emerge, they can dwell in the closed housing, e.g., resting on the inner surfaces of the housing and/or the container, and/or feed on the water and nutrients therein, until they exit from one or more openings 723 (e.g., on the housing lid 721) in the housing, for instance as shown in FIG. 7. The housing and/or container can be designed to have structures 725 (e.g., notches, grooves, passageways, or the like) that permit and/or facilitate exit through the openings 723, while the housing remains closed via lid 721 engaging locking mechanism 727 on the housing and/or the container. In some aspects, the housing can be opened via releasing the engagement between the lid and the locking mechanism. In some embodiments of the method, between about 100 and 10,000, between about 500 and 5,000, or between about 1,000 and 2,000 adults of the aquatic insect emerge in the container in the closed housing and exit the closed housing. In some embodiments, the adults that emerge comprise males and females. In some embodiments, the majority of adults that emerge are male. In some embodiments, at least about 10%, at least about 20%, at least about 30%, at least about 40%, at least about 50%, at least about 60%, at least about 70%, at least about 80%, at least about 90%, at least about 95%, at least about 99%, or 100% of the adults that emerge are male.

In any of the embodiments herein, the adults can be genetically engineered and the method can further comprise allowing the adults to mate with the aquatic insects of the same species in the wild. In some embodiments, the adults that emerge from the apparatus disclosed herein comprise a gene expression system capable of producing a protein having a lethal, deleterious, or sterilizing effect, optionally wherein the gene expression system is a sex-specific gene expression system, optionally wherein the gene expression system and/or protein is activated in the absence of a substance, optionally wherein the substance is a chemical ligand, optionally wherein the chemical ligand is tetracycline. In some embodiments, allowing the adults that emerge from the apparatus disclosed herein to mate with the same species in the wild results in at least some offspring that inherit the gene expression system, thereby suppressing a population of wild insects of said same species. In some embodiments, suppressing the population of wild insects results in reduced transmission of human disease via the population of wild insects; and/or reducing, inhibiting, or eliminating crop damage caused by the population of wild insects.

In some embodiments, the aquatic insect of any of the embodiments herein can be a mosquito. In some embodiments, the mosquito is of the genus Stegomyia, Aedes, Anopheles, Culex or Toxorhynchites. In some embodiments, the mosquito is Aedes aegypti, Aedes albopictus, Culex pipiens, Culex quinquefasciatus, Anopheles stephensi, Anopheles albimanus or Anopheles gambiae.

In particular embodiments, provided herein is a cost effective and repeatable release solution for deploying male insects such as mosquitoes at a high density, e.g., at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1,000, at least about 1,150, at least about 1,200, at least about 1,250, at least about 1,300, at least about 1,350, at least about 1,400, at least about 1,450, at least about 1,500, or more than 1,500 male mosquitoes per unit device per week per hectare. In some embodiments, multiple units of a device is placed per hectare, for instance, two, three, four, five, or more units of the device per hectare. The number of units of the device per hectare can be adjusted as needed, for instance, based on the number of eggs per unit and the mosquito population size in a target geographical area. In some embodiments, multiple units of a device can be used in the same geographical area over the same period of time, for instance, one, two, three, four, or more weeks, and the multiple units can be used simultaneously or in any suitable temporal order. In particular embodiments, using a rearing device or apparatus containing insect eggs, a method disclosed herein comprises deploying insects (e.g., male mosquitoes) at a dose (e.g., a minimum target dose), for instance, at between about 800 and about 1,500, between about 800 and about 1,000, or between about 1,000 and about 1,500 adult insects (e.g., male mosquitoes) per week, for instance, using one, two, three, four, five, or more units of the device per hectare. In particular embodiments, a unit of a rearing device or apparatus containing insect eggs disclosed herein is configured to deploy between about 300 and about 500, between about 600 and about 800, or between about 1,000 and about 1,500 adult insects (e.g., male mosquitoes) per unit device or apparatus during the used lifetime of the device or apparatus and/or the receptacle containing the eggs. Examples of devices with target yields at 1,250, 750, or 375 per unit device are shown in Table 1.

In any of the embodiments herein, the method may comprise using a plurality of the apparatuses disclosed herein, for example as a single-point deployment, distributed over multiple locations in an area, and/or distributed at a single time point or over multiple time points. For instance, the mapping unit disclosed herein may be used to provide a distribution of the plurality of the apparatuses over the area, such as an optimized spatial pattern of the apparatuses based on results of mosquito population control in a similar area. For instance, the plurality of the apparatuses can be arranged in a grid.

In any of the embodiments herein, the apparatus disclosed herein can be used at multiple locations in an area for deployment of adult mosquitoes in the area, as well as in combination with multiple other apparatuses in a network of apparatuses. The network of apparatuses can be used in the same relatively small area, especially in cases where a high density of apparatuses is necessary to achieve a target mosquito number per week per hectare. For instance, higher human density areas such as urban areas may require higher mosquito release numbers per unit area because they may have higher natural mosquito population sizes. In other embodiments, the network of apparatuses can be used in different areas (e.g., adjacent and optionally overlapping areas) such that collectively they cover a larger area. The number of apparatuses for a given area can be determined, for example, based on a variety of factors including but not limited to (i) the human population and mosquito population density in the release area, (ii) mosquito eggs per unit apparatus, (iii) the number of release points in the release area, and (iv) local cost of labor. In some embodiments, a method disclosed herein utilizes a number of apparatuses that minimize release points per unit area and operational intensity while overflooding sufficiently, especially for areas with high pest pressure. In some embodiments, using one or more devices disclosed herein, the number of genetically engineered males that are released is greater than the number of wild males in a target geographical area. The ratio of genetically engineered males that are released to wild males can be adjusted as needed, for instance, based on the fitness of the released males compared to the wild ones. In particular examples, a minimum target does of about 1,250 male mosquitoes per week and a minimum target yield of about 1,250 male mosquitoes per unit apparatus are used. In some embodiments, a method disclosed herein minimizes the number of units deployed to achieve operational efficiency, e.g., for areas having lower pest pressure. In particular examples, a minimum target does of about 750 male mosquitoes per week and a minimum target yield of about 750 male mosquitoes per unit apparatus are used. In some embodiments, a method disclosed herein can be used to lower release target for small target treatment areas, such as a house. In some examples, two units of the apparatus can be in operation at a time with one releasing adult mosquitoes. In particular examples, a minimum target does of about 125 male mosquitoes per week and a minimum target yield of about 250 male mosquitoes per unit apparatus are used.

In any of the embodiments herein, the method can further comprise interfacing with a potential user or a user of the apparatus and/or receptacle. In some of any embodiments, the method is implemented on a system comprising a front end which may be any suitable front end. In some embodiments, the front end is a native application (app). In some examples, frontend and cloud services can be different for different apps based on developer expertise. In some embodiments, the native app is a website and/or a mobile app (e.g., Google, Android, iOS). In some embodiments, the front end is a JavaScript framework. In some embodiments, the front end platform may link the app to any suitable cloud service. In some embodiments, the front end is Angular. In some embodiments, the cloud service is Azure. In some embodiments, the cloud service is AWS. In some embodiments, the system further comprising a backend. In some embodiments, the backend is a cloud service. In some embodiments, the back end is a cloud backend service. In some embodiments, the cloud backend service comprises data storage, security and processing.

In some embodiments, one or more steps of the methods disclosed herein are implemented on an app, for example, a preconfigured app. In some embodiments, the preconfigured app comprises a user-friendly interface for a computer or mobile device, such as a companion web app. In some embodiments, the method comprises creating a user account and/or sign-in credential. In some embodiments, the method comprises receiving sign in data from a returning user, such as a user name/username or email address and a password. In some embodiments, the method comprises recovering a forgotten password.

In some embodiments, the method comprises creating one or more option(s) for a product and/or a service for in-app purchases. In some embodiments, the method comprises displaying the product and/or service for purchase, such as a description of the selected product and/or service, the price of the selected product and/or service, and the total cost associated with the same. In some embodiments, the method comprises offering one or more payment option(s). In some embodiments, the method comprises retrieving and displaying tracking data on the shipped product(s).

In some embodiments, the method comprises receiving user input data. In some embodiments, the method comprises receiving previous customer use data. In some embodiments, the method comprises receiving user input information about an area, for example, a local area's mosquito control activities. In some embodiments, the method comprises receiving user input data from historic records. Non-limiting examples of historic records include previous surveys, data from municipalities, and/or mosquito collections.

In some embodiments, the method comprises receiving user input disease data from health districts and/or hospitals. In some embodiments, the method comprises receiving user input demographic data on human populations. In some embodiments, the method comprises receiving user input apparatus and/or receptacle data. In some embodiments, the method comprises receiving user input release data.

In some embodiments, the method comprises receiving user to input data on current and past trapping data. In some embodiments, trapping data comprises data from egg traps. In some embodiments, trapping data comprises data from adult traps. In some embodiments, method comprises receiving user input of locations that have previously been treated, are currently being treated, and/or where release is ongoing. In some embodiments, the method comprises receiving user input satellite imagery, area locations, population/housing density, and/or polygons. In some embodiments, the method comprises receiving user input information about mosquito production and/or quality control, for example, batch release data. In some embodiments, the method comprises receiving user input information on weather, for example, wind, rainfall, and temperature.

In some embodiments, the method comprises creating and displaying a mapping unit. In some embodiments, the mapping unit comprises a system to process data. In some embodiments, the mapping unit may comprise any GIS solution, for instance, ArcGIS. In some embodiments, the mapping unit comprises a predictive model. In some embodiments, the predictive model is based one or more factor(s). In some embodiments, the one or more factor(s) comprises weather and/or climate data. In some embodiments, the one or more factor(s) comprises ongoing release data from the targeted area(s).

In some embodiments, the mapping unit comprises modelling of distribution for areas without mosquito traps. In some embodiments, the mapping unit comprises modelling of distribution based on trap and weather data from similar areas. In some embodiments, the mapping unit ensures resolution of spatial granularity of data to create adjacent or overlapping polygons, enabling creation of real-time distribution mapping.

In some embodiments, the mapping unit displays a graphical image of apparatus and/or receptacle location(s) and apparatus and/or receptacle status within an area selected by the user. In some embodiments, apparatus and/or receptacle status comprises an indicator, such as a color and/or an icon, of whether the apparatus and/or receptacle has been deployed, when the cartridge of the apparatus and/or receptacle should be changed, and/or when whether the apparatus and/or receptacle should be disposed of. In some embodiments, the mapping unit displays a graphical image of apparatus and/or receptacle locations and/or free spots within an area selected by the user. In some embodiments, more than one area can be selected using the mapping unit. In some embodiments, the mapping unit provides the user with recommendations. In some embodiments, the mapping unit provides the user with recommendations on dosage for one or more area(s) selected by the user. In some embodiments, the mapping unit provides the user with recommendations on placement of apparatus and/or receptacles for one or more area(s) selected by the user.

In some embodiments, the method comprises creating outputs in more than one language, for example, multilingual outputs. In some embodiments, the multilingual outputs are in Spanish, Tagalog, Portuguese, French, English, and/or other languages. In some embodiments, the method comprises creating and displaying a GUI for the user to monitor purchased products, apparatus and/or receptacle location, pending orders, and to use a QR code scan function. In some embodiments, scanning the QR code provides the user with product- or purchase-related information, such as shipping status, location tracking, and/or product status and expiration.

In some embodiments, the method comprises creating and displaying instructions for use of the apparatus and/or receptacle. In some embodiments, the instructions are step-by-step instructions or tutorials. In some embodiments, the method comprises creating and displaying tutorials for installing the apparatus and/or receptacle, such as installing the apparatus and/or receptacle as a standalone unit or attached to a surface. In some embodiments, the tutorials comprise information on installing mounting brackets, attaching the apparatus and/or receptacle, securing the apparatus and/or receptacle, closing the lid of the apparatus and/or receptacle, and opening the lid of the apparatus and/or receptacle. In some embodiments, the method comprises creating and displaying instructions for insect management, such as insect management after installing the apparatus and/or receptacle. In some embodiments, the method comprises creating and displaying user tutorials or answers to common questions, such as a help section. In some embodiments, the method comprises an option for displaying the tutorial(s).

In some embodiments, the method comprises receiving, processing, and/or displaying information on the location of one or more apparatus and/or receptacle(s) in an area. In some embodiments, the method comprises receiving, processing, and/or displaying information on the performance of the apparatus and/or receptacle(s) in the area. In some embodiments, the method comprises receiving, processing, and/or displaying information on the location of one or more apparatus and/or receptacle(s) and the performance of the apparatus and/or receptacle(s) in the area, for example, the effect on the native mosquito population.

In some embodiments, the method comprises receiving information about local area's mosquito control activities. In some embodiments, the method comprises receiving treatment rates and/or treatment failures in a particular area or location. In some embodiments, the method comprises analyzing the received information and displaying a value, such as a percentage, for the number of apparatus and/or receptacle(s) deployed and/or a value describing the area covered, such as a protected area, by the apparatus and/or receptacle(s).

In some embodiments, the method comprises creating one or more alert(s) or notification(s), such as in the form of an update or a prompt. In some embodiments, the one or more alert(s) or notification(s) is an actionable notification. In some embodiments, the one or more alert(s) or notification(s) is based on the user's activity, for example, purchasing activity.

In some embodiments, the one or more alert(s) or notification(s) inform the user on events based on aspects of insect life cycle. Non-limiting examples of such events include egg expiration dates, time from apparatus and/or receptacle activation to mosquito maturity and release.

In some embodiments, the one or more alert(s) or notification(s) inform the user of time-sensitive actions. Non-limiting examples of time-sensitive actions include installation tasks, cartridge expiry date and/or when to change the internal cartridge, timing on replacement orders, such as when to re-order or re-stock consumables.

In some embodiments, the method comprises creating an interface to manage actionable notifications, for example, a task list and one or more options for assigning a task. In some embodiments, the method comprises creating one or more alert(s) or notification(s) related to a task, such as when a task should be performed and to whom a task is assigned. In some embodiments, the app provides the user with one or more recommendation(s). In some embodiments, the one or more recommendation(s) relates to dosage, such as a dosage recommendation based on an area selected by the user.

In some embodiments, described herein is a method of collecting distribution data from a method of distribution of an apparatus and/or a receptacle. In some embodiments, the distribution data is collected by a server. In some of any embodiments, the server is a service provider's server.

In some embodiments, the method of distribution comprises distribution of an apparatus or device or components thereof (e.g., a receptacle) disclosed herein via an aerial vehicle. In some embodiments, the method of distribution comprises delivery by plane. In some embodiments, the method of distribution comprises distribution of a device or components thereof disclosed herein via a ground vehicle. In some embodiments, the method of distribution comprises delivery by car, truck, van, or the like. In some embodiments, the method of distribution comprises distribution of the apparatus and/or the receptacle from a vehicle to one or more predetermined static point(s). In some embodiments, the method of distribution comprises distribution of the apparatus and/or the receptacle from a drone or other aerial vehicle to one or more static point(s). In some embodiments, the method of distribution comprises distribution of the apparatus and/or the receptacle by hand.

In some embodiments, the method of distribution repeats or recurs after an interval of time, such as in a subscription. In some embodiments, the interval of time is one week, two weeks, three weeks, four weeks, one month, two months, three months, four months, five months, or six months. In some embodiments, the subscription comprises monthly delivery of internal box components, e.g. distribution of box contents (e.g., insect egg pots, diet) separately from boxes.

In some embodiments, provided herein are smart apparatus and/or receptacles that both release insects and collect environmental and other data. In some embodiments, the distribution data comprises an index of local infestation. In some embodiments, the distribution data comprises an index of local infestation without use of ovitraps and/or mosquito traps. In some embodiments, the distribution data comprises an index of strain performance.

In some embodiments, the server collects operational data. In some of any embodiments, the server is a service provider's server. In some embodiments, operational data comprises delivery time, resources, the number of apparatus and/or receptacle(s) delivered, the type of apparatus and/or receptacle(s) delivered, and/or one or more delivery location(s).

In some embodiments, operational data comprises the number of apparatus and or receptacle(s) distributed over a period of time. In some embodiments, operational data comprises the number of apparatus and or receptacle(s) distributed over, for example, one week, two weeks, three weeks, four weeks, one month, two months, three months, four months, five months, or six months. In some embodiments, operational data comprises corresponding coverage over an area.

In some embodiments, the server collects operational business development analytics. In some embodiments, business development analytics comprises information related to customer interests. In some embodiments, operational data may influence contemplated production targets, improvement initiatives, and quality control strategy.

In some embodiments, the server collects data prior to the release of an aquatic insect, for example, a mosquito. In some embodiments, the data is used to evaluate and optimize one or more condition(s). In some embodiments, the server collects transportation data. Non-limiting examples of transportation data include temperature during transport, humidity during transport, duration of transport, product integrity, and GPS tracking during transport. In some embodiments, the data is used to evaluate and optimize transport conditions and/or transport efficiency.

In some embodiments, the server collects rearing parameters from field deployed apparatus and/or receptacle(s). Non-limiting examples of rearing parameters include larvae numbers, larvae density, development stage after what time, water quality, feed consistency, remaining feed, pupae numbers after a certain time, adult numbers, adult viability on the water and/or in flight. In some embodiments, rearing parameters are analyzed. In some embodiments, results of the analysis, will inform adjustments to the production of the mosquito rearing apparatus and/or receptacles and components thereof.

In some embodiments, the server collects climate and/or weather data. In some embodiments, the server collects data on the number of released insects. In some embodiments, the server collects the location of releases (spatial data) and time of releases (temporal data). In some embodiments, the server collects pictures of the environment, for example, conditions for insect breeding, such as mosquito breeding. In some embodiments, the server collects trap data, such as data from Ovitrap and BG traps. In some embodiments, the server collects data on aquatic insect activity, for example, viral hotspots and/or mosquito breeding hotspots. In some embodiments, the server collects data on dengue and/or Aedes hotspots. In some embodiments, the server collects security and/or operational data. In some embodiments, the collected data is provided by a mosquito control authority or a health authority. local area's other ongoing mosquito control activities. In some embodiments, data is used to inform subsequent releases.

In some embodiments, the server collects imagery of releases/surveillance. In some embodiments, the server collects trap data, for example, from Ovitraps. Non-limiting examples of trap data include abundance, egg numbers, wild-type and genetically engineered numbers, ratios between the two, species identification, genetically engineered efficacy (female death), male numbers, female numbers, ratio of male to female, genetically engineered and wild-type numbers, their ratio, species identification, genetically engineered efficacy, for example, the absence of genetically engineered female adults. In some embodiments, the server collects real-time download & integration of surveillance data from one or more trap(s), for example a BG trap. In some embodiments, the server collects data on released insect health, longevity and/or persistence. In some embodiments, the server collects security and/or operational data to inform subsequent releases. In some embodiments, the server collects epidemiological data. In some embodiments, the server collects tracking data of arboviruses cases in treated areas before and after deployment. In some embodiments, the server collects customer satisfaction data and/or customer reviews.

In some of any embodiments, the methods disclosed herein are performed with an app. In some embodiments, the app is or receives data from a smart device. In some embodiments, data from more than one app and/or smart device is received and processed.

The present disclosure, in some embodiments thereof, relates to methods, non-transitory computer-readable media, and systems for mission planning relating to arthropod population control, and more particularly, but not exclusively, to the deployment of eggs, pupae, larvae, and/or adults of bred arthropods in a geographical region, such that adults of the bred arthropod emerge from the eggs, pupae, and/or larvae and/or exit the apparatus to mate with adults of the opposite sex in the wild population. In some embodiments, the eggs, pupae, and/or larvae can give rise to genetically engineered arthropods, such as genetically engineered male insects. In some embodiments, the eggs, pupae, larvae, and/or adults of the bred arthropod can be sterile or sterilized, and yet capable of mating or giving rise to adults capable of mating with adults of the opposite sex in the wild population. The methods, compositions, and/or devices disclosed herein can be used to control vector-borne diseases transferred by mosquitoes, such as Dengue, Malaria, Chikungunya and others, which can be carried and spread by a bite from a female mosquito.

In some embodiments, the bred arthropod can be modified laboratory produced mosquitoes that are released into the wild. The laboratory produced mosquitoes can be provided with characteristics that help fight the spread of the disease. In some embodiments, the bred arthropod can be sterile male mosquitoes, e.g., as a result of being treated by radiation at some point in their life cycle. Female mosquitoes tend to mate only once, so an environment of sterile males can dramatically reduce the population. In some embodiments, the bred arthropod can be genetically modified male mosquitoes. In some embodiments, the genetically modified male mosquitoes, when mating with a wild female, do not lead to offspring that successfully reaches adulthood.

Mosquitoes are fragile insects, and a problem arises when trying to store, transport and release the modified male adults in the very large numbers and over very large areas that are needed to make a significant difference in order to control the wild population.

In some embodiments, the methods disclosed herein can be used to deploy large numbers of bred arthropod (e.g., radiation sterilized or genetically modified male mosquitoes) that can be delivered in various climate and terrain conditions. Due to the difficulties of handling the often fragile arthropod, the methods disclosed herein can be used to allocate and deploy adults of the laboratory grown sterile males for maximum effect.

In some embodiments, the methods disclosed herein can be used to deploy large numbers of bred arthropod in hotspots or a geographic region comprising hotspots, thus the majority of bred arthropod adults released are likely to meet up with the wild populations. For instance, mosquito populations do not cover uniform areas and hotspots of mosquito breeding can be around stagnant water. In some embodiments, the ground distribution of mosquitoes can be used to inform the placement of an apparatus disclosed herein. In some embodiments, one or more locations in a geographic location can be selected, and the one or more locations can be adjusted and/or the apparatus can be placed in one or more additional locations, for instance, to account for the amount of drift in the distribution of wild populations as well as the distribution of bred adults once they emerge as adults and exit the apparatus.

In some embodiments, the methods disclosed herein relate to planning and executing release of eggs, pupae, larvae, and/or adults of bred arthropods in a way that makes most effective use of time and resources. In particular, wild arthropod populations, which are unevenly distributed on the ground, can be duly serviced with suitable planning. Furthermore, weather conditions such as wind speed can be taken into account to provide efficient distribution.

In some embodiments, the ground distribution of the population is surveyed. The survey information can be provided in machine-readable format, such as a density distribution on a map. In some embodiments, resources are allocated in accordance with the distribution. In some embodiments, the population density may be translated into an aerial release map.

In some embodiments, provided herein is a system for distribution of eggs, pupae, larvae, and/or adults of bred arthropods (e.g., insects) over a geographical area, comprising: a distribution mapping unit configured to use available population density data of a wild arthropod population to generate a distribution map showing said population density at a desired resolution level; a release mapping unit configured to apply distribution parameters to the distribution map to form a release map, where the distribution parameters describe effects of distribution on actual arthropods. An embodiment may comprise a ground based data gathering unit configured to obtain population density data of a wild arthropod population around said geographical area. In an embodiment, said ground-based data gathering unit comprises an arrangement of traps over said geographic area and an interpolation unit configured to use measurements taken from said traps to assign population density numbers to cells at said resolution level. In an embodiment, said interpolation unit is configured to assign to each cell a number based on insect captures at neighboring traps, the captures at each trap being inversely weighted for distance of the respective trap. In an embodiment, said interpolation unit is configured to assign to each cell a number being an average between each trap within the cell. In an embodiment, the arthropod to be released are sterile male insects. In an embodiment, the insects to be released are mosquitoes.

In some embodiments, the system comprises an update unit configured to obtain additional data about said wild population following distribution and provide an updated distribution plan. The updated plan may be obtained based on additional ground measurements, or on a model of how the arthropods have been distributed.

According to a second aspect of the present disclosure there is provided a method of preparing a distribution program for arthropods, comprising: obtaining distribution data of a wild population of arthropods; obtaining distribution parameters; generating a population density map; generating a release map by modifying said population density map in accordance with said distribution parameters; and generating a path using said release map, the path defining dosage of arthropods to be placed at respective locations along said path, e.g., for release of adult arthropods. The arthropods can be placed in the geographic region as eggs, pupae, larvae, and/or adults. The method may comprise defining one or more locations (e.g., release points or points for placing an apparatus comprising eggs) based on GPS coordinates of a ground-based population monitoring trap, and then at each release point basing a number of arthropods to be released on captures at the corresponding ground-based monitoring trap, so that the arthropod release matches the identified population.

The present embodiments relate to planning and executing release of arthropods in a way that makes most effective use of time and resources. In some embodiments, traps can be used to map a population distribution including density of wild arthropods. In some embodiments, a population density map is then constructed of the arthropod population in question. The arthropod population in question may be considered as a whole, and resources can be allocated on the basis of the population density map.

In some embodiments, a method disclosed herein comprises building a release/placement map over a region which shows different densities per each of a plurality of area units into which the region is divided, where the density data is based on a series of traps set out across the region. In some embodiments, data is integrated from the various sources such as ground traps, densities of local infection rates from medical services, and/or municipal information regarding historical spraying, disease hotspots, locations of rubbish dumps/cisterns/citations for fly tipping, etc. In some embodiments, the release/placement map is generated based on forecast using information regarding the arthropod; information regarding conditions in and/or outside the apparatus; information regarding weather and climate in the geographic region; terrain information in the geographic region; and/or information regarding human population and/or activity in the geographic region.

In some embodiments, a method disclosed herein comprises mission planning to provide the ability to foresee the progress of the mosquitoes and thus suggest when and where to place the arthropod eggs, pupae, larvae, and/or adults. In some embodiments, the maps may include dynamic information about the movement of the arthropod, including wild populations and/or bred arthropods. Regularly updated maps may show that the arthropods are moving in a particular direction and this can be taken into account when and where to place the arthropod eggs, pupae, larvae, and/or adults.

In some embodiments, the density information is made as comprehensive as possible since sporadic locations of arthropods (e.g., mosquitoes) may not provide enough information. Once it is known how many bred arthropods are required and when and where they are needed, then the release and/or placement of eggs, pupae, larvae, and/or adults bred arthropod may be managed accordingly. In some embodiments, the bred arthropod eggs, pupae, larvae, and/or adults to be placed in a geographic region are or give rise to sterile males, which are released to control the wild population.

In some embodiments, a ground based data gathering unit is used to obtain population density data of a wild insect population around the geographical area, e.g., using one or more traps and/or smart counting devices. An interpolation unit may use measurements taken from the traps to assign population density numbers to cells at the resolution level. The interpolation unit may assign to each cell a number based on insect captures at neighboring traps, the captures at each trap being inversely weighted for distance of the respective trap. In some embodiments, the interpolation unit may assign to each cell a number being an average between each trap within the cell. The cells may be equal area release cells.

In some embodiments, an update unit may obtain additional data about the wild population following distribution and provide and updated distribution plan. A method of preparing a distribution program for arthropods can involve obtaining distribution data of a wild population such as the wild mosquito population. Distribution parameters including distribution resolution levels of one or more available distribution vehicle are obtained. A population density map is obtained at the resolution level consistent with the available vehicle, and then a release map is generated by modifying the population density map in accordance with the distribution parameters. From the release map a path is defined which includes dosages of insects at the various locations along the path.

In some embodiments, traps may be used to determine wild population densities and determine how many bred arthropods are to be released in a given area that is on a given grid. The area is identified and is divided into a number of grid. Traps are allocated over the grid to give the best coverage that is practical for the geographical area. The traps are monitored at regular intervals. The interval may be a preset frequency. If traps with electronic monitoring are available than real time monitoring may be provided.

The numbers of trapped arthropods can provide a population sample, as well as an estimate of the wild population in the area. In some embodiments, traps may give relative densities of the population, and a more thorough analysis at one particular location may allow for a translation factor to get from the trap to an estimate of the actual population density, or existing data may be available. Having obtained the numbers from the traps, the trap numbers can be translated into estimates of numbers of wild arthropods per grid square, so that the numbers of arthropods to be released can be determined. For example, the number of wild arthropods per unit area found in the traps could be multiplied by 5, 10, 15, 20, or more to give the number of the sterile arthropods to be released in that grid square. Different policies may apply when extrapolating the number of actual wild arthropods from the trap results, but that there is such a factor, and different factors may apply in different circumstances.

Depending on the capability of the release device (e.g., comprising bred arthropod eggs, pupae, larvae, and/or adults), the release rate can be calculated as per a required number of arthropods per unit of time (e.g., the number of adults per day that exit an apparatus after one week, two weeks, three works, or four weeks from egg hydration), or required number of arthropods per unit area (e.g., hectare or acre). Hence a release method may enable a dosage release—and a release map may require different dosage at different places. In some embodiments, estimating the density may involve extrapolating the actual release dosage required per hectare and/or per release point from sporadic traps. In some embodiments, the method discloses herein comprises estimating densities within the area of interest, which are at different locations, after which the densities may be translated into the release map. Exemplary methods for estimating densities within an area of interest include but are not limited to those described in U.S. Pat. No. 10,945,410 B2 and Application Publication No. US 2021/0195866, which are incorporated herein by reference in their entireties for all purposes.

### VII. TERMINOLOGY

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” or “fewer than” or “fewer or equal to” or the like precedes the first numerical value in a series of two or more numerical values, the term “no more than,” “less than,” or “less than or equal to” or “fewer than” or “fewer or equal to” applies to each of the numerical values in that series of numerical values. For example, less than or equal to 3, 2, or 1 is equivalent to less than or equal to 3, less than or equal to 2, or less than or equal to 1.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### VIII. EXAMPLES

The following examples are provided to illustrate embodiments of the present disclosure but they are by no means intended to limit its scope.

### Example 1. Setting Up a Mosquito Rearing Device Assembly

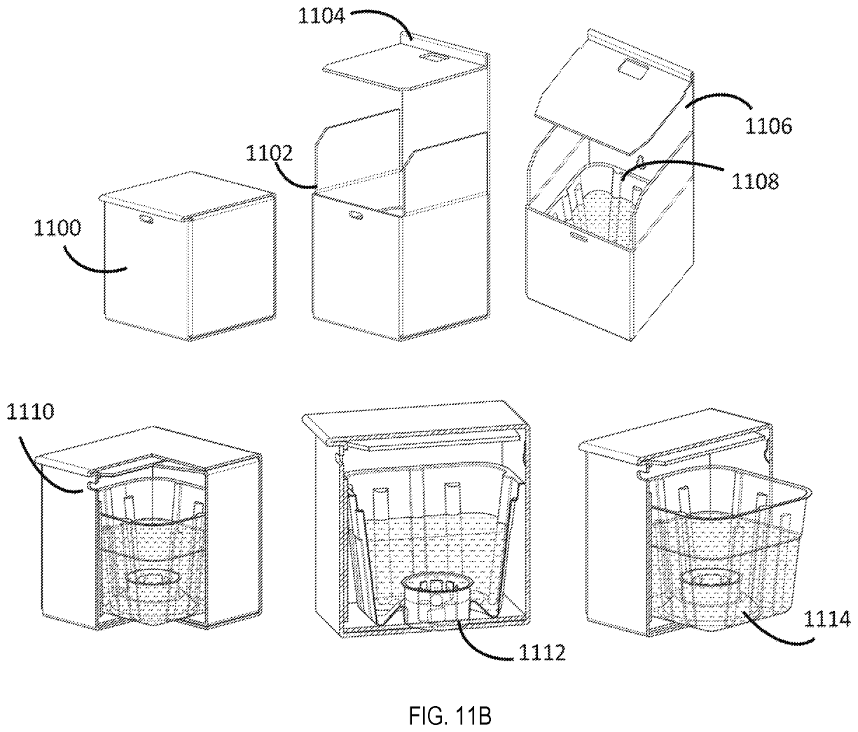

A mosquito rearing device was assembled from a kit comprising a housing, a container, a receptacle with an egg pot, insect eggs, and water preservatives following steps outlined in FIG. 11A.

Firstly, based on the local environment, a desirable location for the mosquito rearing device described herein was recommended by an app provided with the purchase of the device kit. Following the recommendations, the assembly kit was positioned in the desired location and the lid on the housing was opened by pressing a quick-release safety clip on the lid (Step 1). All consumables included in the housing were taken out to create an empty space inside of the housing. Then the container was inserted into and fixed to the bottom of housing together with capsules containing water preservatives, which were pre-attached onto the base of the container (Step 2). Once the position of container and capsules was confirmed and stabilized, a layer of airtight seal on the receptacle was peeled off (Step 3) and then the receptacle comprising the egg pot and diet section was installed onto the container (Step 4). The installation process was enabled via recesses on the bottom of the container, which allowed easy insertion and secure connection. After confirming that the receptacle was locked onto the container, water was added slowly into the container to the fill line (Step 5) and the lid on the housing was closed (Step 6).

The as-assembled mosquito rearing device can be mounted on a wall or positioned on the ground. Some exemplary locations to place the assembly device include lamp post, tree, walls, or garden ground. Once the container and all other single-use components were used for a period of time suggested by the instruction, they were discarded in standard recycle trash bins and replaced with a new container and fresh consumables for a new cycle of usage.

### Example 2. A Portable Light-Weight Mosquito Rearing Device

To improve the portability of the mosquito rearing device and satisfy the need for small area treatment, a mosquito rearing device was designed and built with light-weight materials and optimized for small area treatment. As shown in FIG. 11B, a mini model 1100 comprises a housing component 1102, a container 1108, and a receptacle 1112 with egg pot and diet section. The housing was made with light-weight cardboard with white exterior surface to reduce solar gain and maintain a temperature (in some examples, between about 20° C. and about 28° C.) that facilitates rearing of aquatic insects. The exterior surface of the housing was coated with a wax layer to prevent or delay water damage to the device. The housing also comprised a cardboard flap lid 1106, which further comprised a cardboard lip 1104 that could be folded to seal the device. A hole 1110 on the upper part of the housing allowed adult mosquitoes to exit the device. The container was made with recyclable PET plastic and had an internal clipping feature 1114 into which the receptacle fitted securely. The size, shape and rigidity of the container were optimized with the housing so that the container stayed stable inside of the device even when the device was hanging above the ground. A receptacle 1112 with egg pot and diet section was inserted into the container and was locked onto the clipping feature to secure the connection. After assembling the housing and installing the receptacle, water was filled to the filling line and the container 1108 was put into the housing. The cardboard flap 1106 was then folded to seal the device and the device was put in a desired shaded area.

### Example 3. Device Performance

To satisfy different needs for pest control, various mosquito rearing device models were designed and built. These devices with varied parameter and performances could satisfy the need of a variety of customers, such as governments, business entities, or individual households/customers, and could be deployed in a variety of environments, such as city parks, industrial sites, hotels, residential gardens, café, or shop. The yield, spoilage rate, service interval, required boxes per HA, release points per HA, shelf life, and advantages of the three models were summarized in Table 1.

TABLE 1
Features of three mosquito rearing devices

| Model No. | 1 | 2 | 3 |
|---|---|---|---|
| Target Yield | 1,250 males/box | 750 males/box | 375 males/box |
| Spoilage | <2% | <2% | <2% |
| Service Interval | 10 days (28-day box) | 14 days (28-day box) | 14 days (28-day box) |
| Required Boxes per HA | 6 | 4 | 2 |
| Release points per HA | 2 | 2 | 1 |
| Shelf Life | 12 weeks | 12 weeks | 12 weeks |
| Features and | Minimized | Reduced deploying | Sustained and |
| Advantages | deploying points per | points per acre | constant release |
| | acre and operational | and/or operational | enabled by two |
| | intensity while | intensity and more | boxes in operation |
| | offering reliable and | suitable for sites | at a time (one to |
| | sufficient release of | with relatively lower | grow males and the |
| | males to overflood | wild population | other one to actively |
| | the wild males and | (e.g., lower pest | release males). Each |
| | achieve efficient pest | pressure) | box being set up |
| | control | | every 14 days. |
| | | | Optimized for lower |
| | | | release target and |
| | | | small target |
| | | | treatment areas |

A yield of 1,250 males/device was set as a target in Model 1 to minimize the release points required per HA. In some examples, yield between 800 and 1,000 males per device was observed, and such slightly lower yields were easily compensated by a small increase in the deployed points. Model 1 was capable of routinely producing about 1,250 males per device. In some examples, the average device performance was about 1250 males per device. A yield of 750 males/device was set as a target in Model 2 to reduce the release points required per HA while offer sufficient pest control at places with relatively lower wild pest population. Model 3 was capable of routinely producing at least 375 males per box, with one box growing male mosquitoes while the other actively releasing males to give a relatively constant release over time.

In some cases, the device lifetime was 28 days, with a service interval from 10 days to 14 days, so that significantly less user's operational intensity would be warranted. The spoilage rate was designed to be less than 2%, which was achieved in the three models. Slightly higher spoilage rate can be easily compensated by early-adopter customer care. In some cases, the devices had a 12-week shelf-life to facilitate distribution and simplify the logistics for sale.

The present disclosure is not intended to be limited in scope to the particular disclosed embodiments, which are provided, for example, to illustrate various aspects of the disclosure. Various modifications to the compositions and methods described will become apparent from the description and teachings herein. Such variations may be practiced without departing from the true scope and spirit of the disclosure and are intended to fall within the scope of the present disclosure.

1. An apparatus for rearing an aquatic insect, comprising:
a container comprising a bottom wall and a side wall extending from the bottom wall, and wherein the bottom wall and the side wall are configured to form a chamber for holding a hydrating composition;
a receptacle configured to hold eggs of the aquatic arthropod (e.g., insect) and engage the container such that when the hydrating composition is in the chamber, at least a portion of the receptacle contacts the hydrating composition to establish a fluid communication between an inner cavity of the receptacle and the hydrating composition; and
an optional housing configured to house the container.

2. The apparatus of claim 1, wherein the housing comprises a housing body and a lid configured to engage the housing body such that the housing has a closed configuration and an open configuration, wherein the housing in the closed configuration encloses the container.

3. The apparatus of claim 2, wherein the lid is movable relative to the housing body, optionally wherein the lid releasably, rotatably, pivotably, slidably, and/or removably engages the housing body.

4. The apparatus of claim 2 or 3, wherein the lid rotatably engages a first side wall of the housing body and releasably engages a second side wall of the housing body via a releasable locking mechanism.

5. The apparatus of any of claims 1-4, wherein the housing comprises one or more openings configured to allow exit of an adult of the aquatic insect, optionally wherein the housing body and/or the lid comprises one or more openings configured to allow exit of an adult of the aquatic insect.

6. The apparatus of claim 5, wherein the one or more openings are independently between about 2 mm and about 30 mm in at least one dimension, optionally wherein the one or more openings are between about 3 mm and about 20 mm long and/or between about 3 mm and about 20 mm wide.

7. The apparatus of claim 5 or 6, wherein the lid comprises one or more openings which, together with one or more corresponding structures in the housing and/or the container, provide air communication between the chamber and an outside environment when the housing is in the closed configuration, thereby allowing exit of the adult of the aquatic insect into the outside environment.

8. The apparatus of any of claims 5-7, wherein the one or more openings are configured to prevent rain and/or a predator of the aquatic insect from entering the chamber, optionally wherein the one or more openings are downward inclined from inside to outside of the housing.

9. The apparatus of any of claims 1-8, wherein the housing and/or the container comprises a temperature insulating material, optionally wherein the housing body, the lid, the bottom wall, and/or the side wall of the container comprises a temperature insulating material.

10. The apparatus of any of claims 1-9, wherein the housing comprises a shell and a temperature insulating filler inside the shell, optionally wherein the shell comprises polypropylene and the filler comprises polyurethane foam.

11. The apparatus of any of claims 1-10, further comprising a filler between the housing and the container when the container is placed inside the housing, optionally wherein the filler comprises a foam, optionally wherein the foam is a temperature insulating foam disposed on or contacting with an inner surface of the housing and/or an outer surface of the container.

12. The apparatus of any of claims 1-11, wherein the housing, the container, and/or the filler is configured to maintain an average temperature of between about 10° C. and about 40° C. in the chamber and/or the hydrating composition therein.

13. The apparatus of any of claims 1-12, wherein the housing, the container, and/or the filler is configured to maintain an average temperature of between about 15° C. and about 32° C. in the chamber and/or the hydrating composition therein.

14. The apparatus of any of claims 1-13, wherein the housing, the container, and/or the filler is configured to maintain an average temperature of between about 20° C. and about 28° C. in the chamber and/or the hydrating composition therein.

15. The apparatus of any of claims 1-14, wherein inner surfaces of the housing and/or the container are grippable by tarsals of adults of the aquatic insect, optionally wherein the inner surfaces comprise a plurality of indentations and/or protrusions.

16. The apparatus of any of claims 1-15, wherein the container releasably and/or removably engages the housing body and is replaceable, optionally wherein the container comprises a recyclable material.

17. The apparatus of any of claims 1-16, wherein the container comprises one or more adapter structures configured to engage the receptacle and/or one or more additional components, optionally where the one or more adapter structures are on the bottom wall.

18. The apparatus of claim 17, wherein the one or more additional components comprise a water preservative, optionally wherein the water preservative is provided in a capsule clipped into an adapter structure on the bottom wall, optionally wherein the water preservative is provided in a pre-loaded tablet or is to be added in the container and/or hydrating compositions by a user, optionally wherein the water preservative is provided in a pre-loaded sachet or is to be sprinkled in the container and/or hydrating compositions by a user, optionally wherein the capsule, sachet, or tablet comprises a filler substance and/or an effervescent reagent, optionally wherein the filler substance comprises cellulose or calcium phosphate, and/or optionally wherein the effervescent reagent comprises sodium bicarbonate or solvate thereof, adipic acid or solvate thereof, or citric acid anhydrous.

19. The apparatus of any of claims 1-18, wherein the container and/or the hydrating compositions therein comprises a water preservative, optionally wherein the water preservative comprises sorbic acid or a salt or solvate thereof, or a chlorinated hydroxytriazine or a salt or solvate thereof, or citric acid or a salt or solvate thereof, optionally wherein the water preservative comprises potassium sorbate and/or sodium dichloroisocyanurate and/or citric acid.

20. The apparatus of any of claims 1-19, wherein:
the container comprises a volume indicator of the hydrating composition, optionally wherein the volume indicator comprises a fill line on the side wall of the container; and/or
the container comprises a pH regulator, optionally wherein the pH regulator is formulated to maintain an average pH value of between about 5.8 and about 7.5 in the hydrating composition, and optionally wherein the pH regulator is formulated to maintain an average pH value of between about 6.0 and about 7.0 in the hydrating composition, and optionally wherein the pH regulator is formulated to maintain an average pH value of between about 6.3 and about 6.5 in the hydrating composition.

21. The apparatus of any of claims 1-20, which is configured to provide a head space between the hydrating composition and the housing in a closed configuration, when the container is placed inside the housing and the hydrating composition is in the chamber.

22. The apparatus of any of claims 1-21, wherein the hydrating composition comprises or is water, and the volume of the hydrating composition in the chamber is between about 200 mL and about 10,000 mL, optionally wherein the volume of the hydrating composition in the chamber is between about 500 mL and about 1500 mL.

23. The apparatus of any of claims 1-22, wherein the receptacle is partially or fully submerged in the hydrating composition.

24. The apparatus of any of claims 1-23, wherein the receptacle is clipped into an adapter structure on the bottom wall, tied to the bottom wall, and/or held to the bottom wall by a force from above the receptacle.

25. The apparatus of any of claims 1-24, wherein the receptacle comprises an egg section for holding eggs of the aquatic insect.

26. The apparatus of claim 25, wherein the receptacle is configured to provide a fluid communication between the egg section and the hydrating composition when at least a portion of the receptacle contacts the hydrating composition.

27. The apparatus of any of claims 1-26, wherein prior to contacting the hydrating composition, the receptacle maintains an internal relative humidity between about 50% and about 90%, optionally between about 55% and 75%, thereby maintaining viability of the eggs of the aquatic insect.

28. The apparatus of any of claims 1-27, wherein the receptacle comprises a seal configured to form an airtight seal between the inner cavity and an outside environment, optionally wherein the seal seals the egg section and the diet section from the outside environment.

29. The apparatus of claim 28, wherein the seal is removable, optionally wherein the seal comprises foil and is manually removable or optionally wherein the seal is dissolvable in the hydrating composition.

30. The apparatus of any of claims 1-29, wherein the receptacle comprises an internal surface configured to attach to the eggs of the aquatic insect, optionally wherein the internal surface is adhesive or rendered adhesive via spraying and/or painting the internal surface with an adhesive, and optionally wherein the internal surface is on a bottom wall, a side wall, or an internal wall of the receptacle or on an insert or enclosure in the receptacle.

31. The apparatus of claim 30, wherein the internal surface is on a one-sided or two-sided adhesive strip, optionally wherein the two-sided adhesive strip is affixed to a surface enclosing the egg section.

32. The apparatus of any of claims 1-31, comprising a hatch stimulant, optionally wherein the hatch stimulant is in proximity to the eggs in the receptacle and reduces oxygen potential sufficient to trigger a synchronous larval hatch from the eggs.

33. The apparatus of claim 32, wherein the hatch stimulant is dissolvable in water and comprises cysteine, ascorbic acid, or a salt or solvate thereof,
optionally wherein the cysteine concentration in the hydrating composition is between about 10 mM and about 125 mM, optionally between about 15 mM and about 100 mM, and
optionally wherein the ascorbic acid concentration in the hydrating composition is between about 1.5 mM and about 40 mM, optionally between about 3 mM and about 30 mM.

34. The apparatus of any of claims 1-33, wherein the receptacle comprises a diet section for holding diet for the aquatic insect larvae.

35. The apparatus of claim 34, wherein the receptacle is configured to provide a fluid communication between the diet section and the hydrating composition when at least a portion of the receptacle contacts the hydrating composition.

36. The apparatus of claim 34 or 35, wherein the diet section is configured to allow the hydrating composition to wet at least a portion of the diet in the diet section.

37. The apparatus of claim 36, wherein the diet section is configured to allow the hydrating composition to wet the diet in a bottom section of the diet section while maintaining dry diet in a top section of the diet section.

38. The apparatus of any of claims 34-37, wherein the diet is separated from the eggs by a perforated or castellated enclosure.

39. The apparatus of any of claims 34-38, wherein the diet is formulated to support development of the aquatic insect from egg to larva, to pupa, and/or to adulthood.

40. The apparatus of any of claims 34-39, wherein the diet is formulated to minimize spoilage of the hydrating composition.

41. The apparatus of any of claims 34-40, wherein the diet is formulated to dissolve and/or disintegrate in a controlled manner when contacted with the hydrating composition.

42. The apparatus of any of claims 34-41, wherein the diet is formulated as pellets, optionally wherein the diet comprises a single pellet type or multiple pellet types, optionally wherein the single pellet type comprises the same or different diet compositions, and optionally wherein the multiple pellet types comprise the same or different diet compositions.

43. The apparatus of any of claims 34-42, wherein the diet is coated, optionally wherein the coating is configured to control the rate at which the diet dissolves and/or disintegrates when contacted with the hydrating composition.

44. The apparatus of claim 43, wherein the diet comprises a plurality of pellets coated by the same coating or by different coatings, optionally wherein the plurality of pellets comprise the same or different diet compositions.

45. The apparatus of any of claims 34-44, wherein the diet is formulated to provide moisture in the receptacle which is sealed, thereby maintaining an internal relative humidity of the sealed receptacle between about 50% and 90%, optionally between about 55% and 75%.

46. The apparatus of any of claims 1-45, wherein the receptacle is sealed and the sealed receptacle contains between about 0.01 g and about 0.5 g eggs of the aquatic insect, optionally wherein the sealed receptacle contains between about 0.01 g and about 0.02 g, between about 0.02 g and about 0.03 g, between about 0.03 g and about 0.04 g, between about 0.04 g and about 0.05 g, between about 0.05 g and about 0.06 g, between about 0.06 g and about 0.07 g, between about 0.07 g and about 0.08 g, between about 0.08 g and about 0.09 g, between about 0.09 g and about 0.1 g, between about 0.1 g and about 0.15 g, between about 0.15 g and about 0.2 g, between about 0.2 g and about 0.25 g, between about 0.25 g and about 0.3 g, between about 0.3 g and about 0.35 g, between about 0.35 g and about 0.4 g, between about 0.4 g and about 0.45 g, or between about 0.45 g and about 0.5 g eggs.

47. The apparatus of any of claims 1-46, wherein the receptacle is sealed and the sealed receptacle contains between about 1,000 and about 60,000 eggs of the aquatic insect, optionally wherein the sealed receptacle contains between about 1,000 and about 2,000, between about 2,000 and about 3,000, between about 3,000 and about 4,000, between about 4,000 and about 5,000, between about 5,000 and about 6,000, between about 6,000 and about 7,000, between about 7,000 and about 8,000, between about 8,000 and about 9,000, between about 9,000 and about 10,000, between about 10,000 and about 15,000, between about 15,000 and about 20,000, between about 20,000 and about 25,000, between about 25,000 and about 30,000, between about 30,000 and about 35,000, between about 35,000 and about 40,000, between about 40,000 and about 45,000, between about 45,000 and about 50,000, between about 50,000 and about 55,000, or between about 55,000 and about 60,000 eggs of the aquatic insect.

48. The apparatus of any of claims 1-47, wherein at least about 30%, at least about 40%, at least about 50%, at least about 60%, at least about 70%, at least about 80%, at least about 90%, or more of the eggs in the receptacle are hatched.

49. The apparatus of any of claims 1-48, wherein the eggs in the receptacle are free of a bacterium of the genus Wolbachia, optionally wherein the aquatic insect is a mosquito of the genus Aedes, and optionally wherein the mosquito is Aedes aegypti.

50. The apparatus of any of claims 1-49, containing between about 1,000 and about 50,000 larvae of the aquatic insect, optionally wherein the apparatus contains between about 1,000 and about 2,000, between about 2,000 and about 3,000, between about 3,000 and about 4,000, between about 4,000 and about 5,000, between about 5,000 and about 6,000, between about 6,000 and about 7,000, between about 7,000 and about 8,000, between about 8,000 and about 9,000, between about 9,000 and about 10,000, between about 10,000 and about 15,000, between about 15,000 and about 20,000, between about 20,000 and about 25,000, between about 25,000 and about 30,000, between about 30,000 and about 35,000, between about 35,000 and about 40,000, between about 40,000 and about 45,000, or between about 45,000 and about 50,000 larvae of the aquatic insect.

51. The apparatus of claim 50, wherein at least about 10%, at least about 20%, at least about 30%, at least about 40%, at least about 50%, at least about 60%, at least about 70%, at least about 80%, at least about 90%, or more of the larvae of the aquatic insect are male.

52. The apparatus of any of claims 1-51, containing between about 500 and about 25,000 hatched male larvae of the aquatic insect, optionally wherein the apparatus contains between about 500 and about 1,000, between about 1,000 and about 1,500, between about 1,500 and about 2,000, between about 2,000 and about 3,000, between about 3,000 and about 4,000, between about 4,000 and about 5,000, between about 5,000 and about 6,000, between about 6,000 and about 7,000, between about 7,000 and about 8,000, between about 8,000 and about 9,000, between about 9,000 and about 10,000, between about 10,000 and about 15,000, between about 15,000 and about 20,000, or between about 20,000 and about 25,000 hatched male larvae of the aquatic insect.

53. The apparatus of any of claims 1-52, configured to yield between about 250 and about 10,000 adults of the aquatic insect, optionally wherein the apparatus is configured to yield between about 250 and about 500, between about 500 and about 1,000, between about 1,000 and about 1,200, between about 1,200 and about 1,400, between about 1,400 and about 1,600, between about 1,600 and about 1,800, between about 1,800 and about 2,000, between about 2,000 and about 3,000, between about 3,000 and about 4,000, between about 4,000 and about 5,000, between about 5,000 and about 6,000, between about 6,000 and about 7,000, between about 7,000 and about 8,000, between about 8,000 and about 9,000, or between about 9,000 and about 10,000 adults of the aquatic insect.

54. The apparatus of claim 53, wherein at least about 10%, at least about 20%, at least about 30%, at least about 40%, at least about 50%, at least about 60%, at least about 70%, at least about 80%, at least about 90%, at least about 95%, at least about 99%, or 100% of the adults are males.

55. A receptacle, comprising:
a sealable enclosure comprising an egg section comprising eggs of an aquatic insect, and a diet section comprising diet for the aquatic insect, optionally wherein the egg section and the diet section are separated by a barrier with one or more openings; and
a removable seal forming an airtight seal separating the enclosure from an outside environment,
wherein when the seal is removed and at least a portion of the receptacle contacts a hydrating composition, the hydrating composition enters the egg section and the diet section and contacts at least a portion of the eggs and at least a portion of the diet.

56. A receptacle, comprising:
(a) a receptacle base comprising:
(i) a receptacle bottom wall,
(ii) a receptacle side wall extending from the receptacle bottom wall, and
(iii) an enclosure, optionally extending from the receptacle bottom wall,
wherein the receptacle side wall and/or the enclosure forms an egg section and a diet section within the receptacle base, and
wherein the egg section and the diet section are connected;
(b) a receptacle lid comprising a plurality of perforations and configured to engage the receptacle base; and
(c) a receptacle seal configured to form an airtight seal with the receptacle base and/or the receptacle lid, thereby sealing the egg section and the diet section from an outside environment.

57. The receptacle of claim 56, which is configured to hold eggs of an aquatic insect in the egg section, optionally wherein the eggs are between about 0.06 g and about 0.08 g.

58. The receptacle of claim 57, further comprising an adhesive surface on the receptacle side wall, on the receptacle bottom wall, on the enclosure, and/or in the egg section, configured to adhere to eggs of the aquatic insect.

59. The receptacle of any of claims 56-58, which is configured to hold diet for an aquatic insect in the diet section, optionally wherein the diet is formulated to dissolve and/or disintegrate in a controlled manner when contacted with water.

60. The receptacle of any of claims 56-59, wherein the egg section and/or the diet section is configured to hold a hatch stimulant, optionally wherein the hatch stimulant is cysteine or ascorbic acid or a salt or solvate thereof.

61. The receptacle of any of claims 56-60, wherein the egg section and the diet section are separated by the enclosure which is perforated and/or castellated.

62. The receptacle of any of claims 56-61, wherein the enclosure comprises a plurality of panels extending from the receptacle bottom wall, optionally wherein the enclosure and the receptacle side wall form concentric circles.

63. The receptacle of any of claims 56-62, wherein one or more of the plurality of perforations on the receptacle lid are configured to permit passage of water and a larva of an aquatic insect.

64. The receptacle of any of claims 56-63, wherein one or more of the plurality of perforations on the receptacle lid are independently between about 5 mm and about 50 mm in at least one dimension.

65. The receptacle of any of claims 56-64, wherein the receptacle seal forms an airtight seal with the receptacle side wall, and the receptacle lid is inside the receptacle base.

66. A receptacle, comprising:
(a) a receptacle base comprising:
(i) a receptacle bottom wall, and
(ii) a receptacle side wall extending from the receptacle bottom wall;
(b) an enclosure comprising a tubular body and a flange disposed around the tubular body between a bottom end and an upper end of the tubular body,
wherein the tubular body comprises a bottom opening,
wherein the flange comprises a plurality of perforations,
wherein the flange engages the receptacle side wall, the receptacle side wall and/or the tubular body forms an egg section and a diet section within the receptacle base, the egg section and the diet section are connected, and the egg section is connected to a space above the flange via the plurality of perforations on the flange;
(c) a receptacle seal configured to form an airtight seal with the receptacle base, thereby sealing the enclosure, the egg section, and the diet section from an outside environment.

67. The receptacle of claim 66, wherein the enclosure is configured to insert into the receptacle base.

68. The receptacle of claim 66, wherein the enclosure is disposed on or integral to the receptacle bottom wall.

69. The receptacle of any of claims 66-68, wherein the upper end of the tubular body is closed.

70. The receptacle of any of claims 66-69, wherein the tubular body further comprises an upper opening and a closer reversibly closing the upper opening, optionally wherein the closer does not comprise a perforation.

71. The receptacle of any of claims 66-70, which is configured to hold eggs of an aquatic insect in the egg section, optionally wherein the eggs are between about 0.06 g and about 0.08 g.

72. The receptacle of claim 71, further comprising an adhesive surface on the receptacle side wall, on the receptacle bottom wall, on the tubular body, and/or in the egg section, configured to adhere to eggs of the aquatic insect.

73. The receptacle of any of claims 66-72, which is configured to hold diet for an aquatic insect in the diet section, optionally wherein the diet is formulated to dissolve and/or disintegrate in a controlled manner when contacted with water.

74. The receptacle of any of claims 66-73, wherein the egg section and/or the diet section is configured to hold a hatch stimulant, optionally wherein the hatch stimulant is cysteine or ascorbic acid or a salt or solvate thereof.

75. The receptacle of any of claims 66-74, wherein the egg section and the diet section are connected via the bottom opening of the tubular body, optionally wherein a bottom section of the tubular body is perforated and/or castellated.

76. The receptacle of any of claims 66-75, wherein the tubular body and the receptacle side wall form concentric circles.

77. The receptacle of any of claims 66-76, wherein one or more of the plurality of perforations on the flange are configured to permit passage of water and a larva of an aquatic insect.

78. The receptacle of any of claims 66-77, wherein one or more of the plurality of perforations on the flange are independently between at least 1 mm in at least one dimension.

79. The receptacle of any of claims 66-78, wherein the receptacle seal forms an airtight seal with the receptacle side wall, and the receptacle insert is inside the receptacle base.

80. The receptacle of any of claims 66-78, wherein the receptacle seal forms an airtight seal with the receptacle insert, which in turn forms an airtight seal with the receptacle base.

81. A method for rearing an aquatic insect, comprising:
a) inserting a receptacle into a container, wherein:
the container comprises a bottom wall and a side wall extending from the bottom wall, and the bottom wall and the side wall form a chamber for holding water,
the receptacle comprises an egg section holding eggs of the aquatic insect and a diet section holding a diet composition for the aquatic insect, and
the receptacle is affixed to the bottom wall of the container;
b) adding water to the chamber, whereby water enters the receptacle and contacts the eggs and the diet,
thereby allowing the eggs to hatch in the receptacle.

82. The method of claim 81, wherein the receptacle is inserted into the container, followed by adding water to the chamber to a predetermined level.

83. The method of claim 81, wherein water is added to the chamber to a predetermined level, followed by inserting the receptacle into the container.

84. The method of any of claims 81-83, wherein the egg section further comprises a hatch stimulant.

85. The method of any of claims 81-84, wherein the receptacle is fully submerged in water.

86. The method of any of claims 81-85, wherein water enters the egg section and the diet section at the same time, or water enters the egg section prior to entering the diet section, or water enters the diet section prior to entering the egg section.

87. The method of any of claims 81-86, wherein the diet in the diet section is wetted prior to hatching of the eggs in the egg section, or
at least a portion of the diet in the diet section is kept dry until after hatching of the eggs in the egg section, until after larvae exit the receptacle, and/or until after adults of the aquatic insect emerge.

88. The method of any of claims 81-87, wherein the diet dissolves and/or disintegrates in a controlled manner in water, optionally at least about 50%, at least about 60%, at least about 70%, at least about 80%, at least about 90%, or at least about 95% of the diet is not dissolved and/or disintegrated in water within the first two, three, four, five, or six days of contacting the water.

89. The method of claim 88, wherein the controlled release of the diet provides a diet concentration in water that supports development of the aquatic insect and/or reduces spoilage of water compared to a higher diet concentration in water.

90. The method of any of claims 81-89, further comprising placing the container into a housing prior to or after step a) and/or step b), wherein the housing comprises a housing body and a lid configured to engage the housing body such that the housing has a closed configuration and an open configuration.

91. The method of claim 90, wherein the container is placed in the housing, followed by inserting the receptacle into the container, adding water to the chamber, and closing the housing such that it encloses the container.

92. The method of claim 90 or 91, wherein the housing in the closed configuration maintains an average temperature of between about 10° C. and about 35° C. in the chamber and/or water therein, optionally wherein the average temperature is between about 15° C. and about 32° C., or optionally between about 20° C. and about 25° C.

93. The method of any of claims 90-92, wherein the lid comprises one or more openings which, optionally together with one or more corresponding structures in the housing and/or the container, allow exit of adults of the aquatic insect, optionally the one or more openings are downward inclined from inside to outside of the housing.

94. The method of any of claims 90-93, wherein the receptacle, when sealed, contains between about 0.01 g and about 0.5 g eggs of the aquatic insect prior to water contacting the eggs, optionally wherein the receptacle contains between about 0.01 g and about 0.02 g, between about 0.02 g and about 0.03 g, between about 0.03 g and about 0.04 g, between about 0.04 g and about 0.05 g, between about 0.05 g and about 0.06 g, between about 0.06 g and about 0.07 g, between about 0.07 g and about 0.08 g, between about 0.08 g and about 0.09 g, between about 0.09 g and about 0.1 g, between about 0.1 g and about 0.15 g, between about 0.15 g and about 0.2 g, between about 0.2 g and about 0.25 g, between about 0.25 g and about 0.3 g, between about 0.3 g and about 0.35 g, between about 0.35 g and about 0.4 g, between about 0.4 g and about 0.45 g, or between about 0.45 g and about 0.5 g eggs;
wherein the receptacle contains between about 1,000 and about 60,000 eggs, optionally wherein the sealed receptacle contains between about 1,000 and about 2,000, between about 2,000 and about 3,000, between about 3,000 and about 4,000, between about 4,000 and about 5,000, between about 5,000 and about 6,000, between about 6,000 and about 7,000, between about 7,000 and about 8,000, between about 8,000 and about 9,000, between about 9,000 and about 10,000, between about 10,000 and about 15,000, between about 15,000 and about 20,000, between about 20,000 and about 25,000, between about 25,000 and about 30,000, between about 30,000 and about 35,000, between about 35,000 and about 40,000, between about 40,000 and about 45,000, between about 45,000 and about 50,000, between about 50,000 and about 55,000, or between about 55,000 and about 60,000 eggs;
wherein at least about 30%, at least about 40%, at least about 50%, at least about 60%, at least about 70%, at least about 80%, at least about 90%, or more of the eggs in the receptacle are hatched; and/or
the eggs in the receptacle are free of a bacterium of the genus Wolbachia, optionally wherein the aquatic insect is a mosquito of the genus Aedes, and optionally wherein the mosquito is Aedes aegypti.

95. The method of any of claims 90-94, wherein:
the container in the closed housing contains between about 1,000 and about 50,000 larvae after the eggs hatch, optionally wherein the container in the closed housing contains between about 1,000 and about 2,000, between about 2,000 and about 3,000, between about 3,000 and about 4,000, between about 4,000 and about 5,000, between about 5,000 and about 6,000, between about 6,000 and about 7,000, between about 7,000 and about 8,000, between about 8,000 and about 9,000, between about 9,000 and about 10,000, between about 10,000 and about 15,000, between about 15,000 and about 20,000, between about 20,000 and about 25,000, between about 25,000 and about 30,000, between about 30,000 and about 35,000, between about 35,000 and about 40,000, between about 40,000 and about 45,000, or between about 45,000 and about 50,000 larvae after the eggs hatch, optionally wherein hatching of the eggs is synchronous, and optionally wherein at least about 10%, at least about 20%, at least about 30%, at least about 40%, at least about 50%, at least about 60%, at least about 70%, at least about 80%, at least about 90%, or more of the larvae are male; and/or
the container in the closed housing contains between about 500 and about 25,000 male larvae after the eggs hatch, optionally wherein the container in the closed housing between about 500 and about 1,000, between about 1,000 and about 1,500, between about 1,500 and about 2,000, between about 2,000 and about 3,000, between about 3,000 and about 4,000, between about 4,000 and about 5,000, between about 5,000 and about 6,000, between about 6,000 and about 7,000, between about 7,000 and about 8,000, between about 8,000 and about 9,000, between about 9,000 and about 10,000, between about 10,000 and about 15,000, between about 15,000 and about 20,000, or between about 20,000 and about 25,000 male larvae after the eggs hatch, optionally wherein hatching of the eggs is synchronous.

96. The method of any of claims 90-95, wherein between about 250 and about 10,000 adults of the aquatic insect emerge in the container in the closed housing and/or exit the closed housing, optionally wherein between about 250 and about 500, between about 500 and about 1,000, between about 1,000 and about 1,200, between about 1,200 and about 1,400, between about 1,400 and about 1,600, between about 1,600 and about 1,800, between about 1,800 and about 2,000, between about 2,000 and about 3,000, between about 3,000 and about 4,000, between about 4,000 and about 5,000, between about 5,000 and about 6,000, between about 6,000 and about 7,000, between about 7,000 and about 8,000, between about 8,000 and about 9,000, or between about 9,000 and about 10,000 adults of the aquatic insect emerge in the container in the closed housing and/or exit the closed housing.

97. The method of claim 96, wherein at least about 10%, at least about 20%, at least about 30%, at least about 40%, at least about 50%, at least about 60%, at least about 70%, at least about 80%, at least about 90%, at least about 95%, at least about 99%, or 100% of the adults are males.

98. The method of claim 96 or 97, wherein the adults are genetically engineered and the method further comprises allowing the adults to mate with the same species in the wild.

99. The method of claim 98, wherein the adults comprise a gene expression system capable of producing a protein having a lethal, deleterious, or sterilizing effect, optionally wherein the gene expression system is a sex-specific gene expression system, optionally wherein the gene expression system and/or protein is activated in the absence of a substance, optionally wherein the substance is a chemical ligand, optionally wherein the chemical ligand is tetracycline or an analog or derivative thereof.

100. The method of claim 99, wherein allowing the adults to mate with the same species in the wild results in at least some offspring that inherit the gene expression system, thereby suppressing a population of wild insects of said same species.

101. The method of claim 100, wherein suppressing the population of wild insects results in:
(a) reduced transmission of human disease via the population of wild insects; and/or
(b) reducing, inhibiting, or eliminating crop damage caused by the population of wild insects.

102. A method for rearing an aquatic insect, comprising:
a) inserting a receptacle into a container, wherein:
the container comprises a bottom wall and a side wall extending from the bottom wall, and the bottom wall and the side wall form a chamber for holding water;
the receptacle comprises:
a receptacle base comprising: (i) a receptacle bottom wall, (ii) a receptacle side wall extending from the receptacle bottom wall, and (iii) an enclosure, optionally extending from the receptacle bottom wall, wherein the receptacle side wall and/or the enclosure forms an egg section and a diet section within the receptacle base, and wherein the egg section and the diet section are connected,
a receptacle lid comprising a plurality of perforations and engaging the receptacle base, and
a receptacle seal forming an airtight seal with the receptacle base and/or the receptacle lid, wherein the receptacle seal is removed prior to or after inserting the receptacle into the container; and
the receptacle is affixed to the bottom wall of the container;
b) adding water to the chamber, whereby the receptacle is submerged in water which enters the receptacle via the plurality of perforations and contacts the eggs and the diet,
thereby allowing the eggs to hatch in the receptacle, allowing larvae to exit the receptacle via the plurality of perforations into the chamber, and/or allowing adults of the aquatic insect to emerge in the chamber.

103. The method of claim 102, wherein the enclosure extends from the receptacle bottom wall and forms the diet section.

104. The method of claim 103, wherein the egg section is between the enclosure and the receptacle side wall and further comprises a hatch stimulant.

105. The method of claim 103 or 104, wherein the egg section and the diet section are separated by the enclosure which is perforated and/or castellated.

106. The method of any of claims 102-105, wherein the container is in a housing comprising a housing body and a lid configured to engage the housing body such that the housing has a closed configuration and an open configuration, wherein the lid comprises one or more openings which, optionally together with one or more corresponding structures in the housing and/or the container, allow exit of adults of the aquatic insect when the housing is in the closed configuration.

107. A method for rearing an aquatic insect, comprising:
a) inserting a receptacle into a container, wherein:
the container comprises a bottom wall and a side wall extending from the bottom wall, and the bottom wall and the side wall form a chamber for holding water;
the receptacle comprises:
a receptacle base comprising: (i) a receptacle bottom wall, and (ii) a receptacle side wall extending from the receptacle bottom wall,
an enclosure comprising a tubular body and a flange disposed around the tubular body between a bottom end and an upper end of the tubular body, wherein the tubular body comprises a bottom opening, wherein the flange comprises a plurality of perforations, and wherein the flange engages the receptacle side wall, the receptacle side wall and/or the tubular body forms an egg section and a diet section within the receptacle base, the egg section and the diet section are connected, and the egg section is connected to a space above the flange via the plurality of perforations on the flange, and
a receptacle seal configured to form an airtight seal with the receptacle base, wherein the receptacle seal is removed prior to or after inserting the receptacle into the container; and
the receptacle is affixed to the bottom wall of the container;
b) adding water to the chamber, whereby the receptacle is submerged in water which enters the receptacle via the plurality of perforations on the flange, contacts the eggs, and, through the bottom opening of the tubular body, contacts the diet,
thereby allowing the eggs to hatch in the receptacle, allowing larvae to exit the receptacle via the plurality of perforations on the flange into the chamber, and/or allowing adults of the aquatic insect to emerge in the chamber.

108. The method of claim 107, wherein the egg section is between the enclosure and the receptacle side wall and further comprises a hatch stimulant.

109. The method of claim 107 or 108, wherein the egg section and the diet section are separated by at least a portion of the tubular body which is perforated and/or castellated.

110. The method of any of claims 107-109, wherein the container is in a housing comprising a housing body and a lid configured to engage the housing body such that the housing has a closed configuration and an open configuration, wherein the lid comprises one or more openings which, optionally together with one or more corresponding structures in the housing and/or the container, allow exit of adults of the aquatic insect when the housing is in the closed configuration.

111. The apparatus, receptacle, or method of any of proceeding claims, wherein the aquatic insect is a mosquito.

112. The apparatus, receptacle, or method of claim 111, wherein the mosquito is of the genus Stegomyia, Aedes, Anopheles, Culex, or Toxorhynchites.

113. The apparatus, receptacle, or method of claim 112, wherein the mosquito is Aedes aegypti, Aedes albopictus, Culex pipiens, Culex quinquefasciatus, Anopheles stephensi, Anopheles albimanus or Anopheles gambiae.

114. The apparatus, receptacle, or method of any of claims 111-113, wherein the genome of the eggs of the mosquito comprises a splice control sequence capable of directing sex-specific splicing of a polynucleotide encoding a functional gene product, optionally wherein the functional gene product is a tTAV or homolog thereof, a tTAV2 or homolog thereof, a tTAV3 or homolog thereof, or a tTAF or homolog thereof.

115. The apparatus, receptacle, or method of claim 114, wherein the genome of the eggs of the mosquito further comprises a transcription control element that controls transcription by the presence or the absence of a chemical ligand, optionally wherein the transcription control element is a tetracycline-responsive element.

116. The apparatus, receptacle, or method of claim 114 or 115, wherein the sex-specific splicing results in female-specific lethality of the eggs or larvae, pupae, or adults therefrom.

117. The apparatus, receptacle, or method of any of claims 114-116, wherein the splice control sequence is derived from an Actin-4 gene, optionally wherein said Actin-4 gene is an Aedes spp. Actin-4 gene, and optionally wherein said Actin-4 gene is an Aedes aegypti Actin-4 gene (AeActin-4).

118. A method, comprising:
obtaining information of a wild population of an arthropod in a geographic region, optionally wherein obtaining the information comprises forecasting;
selecting a location in the geographic region, a date, and/or a time during the day based on the obtained information;
placing an apparatus comprising eggs, pupae, larvae, and/or adults of bred arthropods at the selected location, time during the day, and/or date; and
allowing adults to emerge from the eggs, pupae, and/or larvae and/or exit the apparatus, wherein the adults mate with the wild population.

119. The method of claim 118, wherein the arthropod is an insect, and optionally wherein the insect is a mosquito.

120. The method of claim 118 or 119, wherein the forecasting comprises using:
information regarding the arthropod;
information regarding conditions in and/or outside the apparatus;
information regarding weather and climate in the geographic region;
terrain information in the geographic region; and/or
information regarding human population and/or activity in the geographic region.

121. The method of claim 120, wherein the information regarding the arthropod comprises:
hatching time, length of the larval stage, length of the pupal stage, time from hatching to adulthood, adult life expectancy, and/or flight range of the species of the arthropod,
one or more diseases transmitted or borne by the species of the arthropod, and/or
crop or livestock damage caused by the species of the arthropod,
optionally wherein the information is a reference or an estimate.

122. The method of claim 120 or 121, wherein the information regarding the arthropod comprises:
the size of the wild population, the density of the wild population, and/or the distribution of the wild population in the geographic region, and/or
the population size, density, and/or distribution of one or more predators of the arthropod in the geographic region,
optionally wherein the information comprises historical information and/or information obtained at the time of the forecasting.

123. The method of any of claims 120-122, wherein the information regarding climate is information regarding season, temperature, rainfall, humidity, and/or wind conditions in the geographic region, optionally wherein the information comprises historical information, a reference or an estimate, and/or information obtained at the time of the forecasting, optionally wherein the information comprises annual or monthly mean temperatures and precipitations in the geographic region.

124. The method of any of claims 120-123, wherein the terrain information is regarding the amount and/or distribution of water bodies in the geographic region, optionally wherein the water bodies comprise stagnant water, rivers, lakes, ponds, tanks, reservoirs, and/or water treatment facilities, and optionally wherein the terrain information is a reference or an estimate.

125. The method of any of claims 120-124, wherein the information regarding human population and/or activity is information regarding the size, density, distribution, demographic information, and/or personal information of the human population in the geographic region, optionally wherein the information comprises historical information, a reference or an estimate, and/or information obtained at the time of the forecasting.

126. The method of any of claims 120-125, wherein obtaining the information and/or the forecasting is performed using one or more devices automatically, semi-automatically, or manually.

127. The method of any of claims 120-126, wherein the forecasting comprises using information obtained from one or more sources, optionally wherein the one or more sources comprise a local source in one or more devices configured to perform the forecasting and/or one or more internet servers.

128. The method of any of claims 120-127, wherein the forecasting is for about a week, about two weeks, about three weeks, about four weeks, or longer from the time of forecasting.

129. The method of any of claims 126-128, wherein the geographic region is selected using one or more devices, optionally wherein the geographic region is regular or irregular in shape.

130. The method of claim 129, wherein the geographic region is selected using the same device(s) for performing the forecast.

131. The method of claim 129, wherein the geographic region is selected using device(s) different from device(s) for performing the forecast.

132. The method of any of claims 129-131, wherein the selected geographic region is provided to device(s) for performing the forecast based on the selected geographic region.

133. The method of any of claims 126-133, wherein the location in the geographic region is selected using one or more devices.

134. The method of claim 133, wherein the location is selected using the same device(s) for performing the forecast and/or the same device(s) for selecting the geographic region.

135. The method of claim 133, wherein the location is selected using different from device(s) for performing the forecast and/or device(s) for selecting the geographic region.

136. The method of any of claims 133-135, wherein the forecasted information is provided to device(s) for selecting the location in the geographic region.

137. The method of any of claims 118-136, wherein a plurality of locations are selected in the geographic region.

138. The method of claim 137, wherein the plurality of locations form a grid in which each location is a node.

139. The method of claim 138, wherein the grid is a regular grid or irregular grid.

140. The method of any of claims 137-139, wherein one or more of the apparatus is placed at each of the plurality of locations.

141. The method of any of claims 118-140, wherein the apparatus is the apparatus of any of claims 1-53.

142. The method of any of claims 118-141, comprising using ground-based data gathering to obtain population density data of the arthropod population in and/or around the geographical region.

143. The method of claim 142, comprising using the obtained population density data for the forecast.

144. The method of any of claims 118-143, comprising delivering the apparatus comprising eggs, pupae, larvae, and/or adults of bred arthropods at or near the location.

145. The method of any of claims 118-144, wherein the apparatus is delivered at or near the location using a ground distribution vehicle or an aerial craft, optionally wherein the aerial craft is a piloted aircraft or a pilotless drone.

146. The method of any of claims 118-145, comprising providing information of the apparatus to a user to place the apparatus.

147. The method of claim 146, wherein the information of the apparatus comprises tracking formation, optionally wherein tracking formation is provided on a map on a device of the user.

148. The method of claim 146 or 147, wherein the information of the apparatus comprises expiry information.

149. The method of any of claims 118-145, comprising providing information of one or more apparatuses that have been placed in the geographic region.

150. A non-transitory computer-readable medium comprising processor-executable instructions to cause a processor to:
obtain information of a wild population of an arthropod in a geographic region, optionally wherein the processor-executable instructions cause the processor to forecast the information;
provide instruction or recommendation to a user for placing an apparatus comprising eggs, pupae, larvae, and/or adults of bred arthropods at one or more locations in the geographic region based on the forecasted information, wherein adults of the bred arthropod emerge from the eggs, pupae, and/or larvae and/or exit the apparatus to mate with the wild population.

151. The method of any of claims 118-149 or the non-transitory computer-readable medium of claim 150, for use in controlling the wild population of the arthropod in the geographic region.

152. The method of any of claims 118-149 or the non-transitory computer-readable medium of claim 150, wherein the adults that exit the apparatus are males only or females only, or include both males and females.

153. The method of any of claims 118-149 or the non-transitory computer-readable medium of claim 150, wherein the adults that exit the apparatus are:
genetically engineered insects that mate with wild insects of the same species of the opposite sex,
sterile or sterilized insects that mate with wild insects of the same species of the opposite sex, optionally wherein the insects are sterilized using radiation, and/or
insects that carry a bacterium of the genus Wolbachia that mate with wild insects of the same species of the opposite sex, optionally wherein the insects are infected with Wolbachia pipientis,
thereby controlling the wild population in the geographic region.

154. The method of any of claims 118-149 or the non-transitory computer-readable medium of claim 150, wherein the arthropod is a mosquito of the genus Aedes, Anopheles, or Culex.

155. The method of any of claims 118-149 or the non-transitory computer-readable medium of claim 150, wherein the apparatus contains eggs of the bred arthropods and no pupae, larvae, or adults of the bred arthropods.
